(19) United States
(12) Patent Application Publication
(10) Pub. No.: US 2025/0282750 A1
(43) Pub. Date: Sep. 11, 2025

(54) METHODS OF MANUFACTURING AN ANDROGEN RECEPTOR PROTEIN DEGRADER

(71) Applicant: Arvinas Operations, Inc., New Haven, CT (US)

(72) Inventors: Chungpin Herman CHEN, Madison, CT (US); Nitinchandra Dahyabhai PATEL, Robbinsville, NJ (US); Hanqing DONG, Madison, CT (US); Maxwell Marco REEVE, Guilford, CT (US); John A. GROSSO, Princeton Jct., NJ (US)

(21) Appl. No.: 19/072,307

(22) Filed: Mar. 6, 2025

Related U.S. Application Data
(60) Provisional application No. 63/562,672, filed on Mar. 7, 2024.

(51) Int. Cl. C07D 401/12

(52) U.S. Cl. CPC C07D 401/12

(57) ABSTRACT

This disclosure pertains to methods of manufacturing Compound A and intermediates in the preparation of Compound A, including methods for the preparation and purification of Compound A, and preparation and purification of such intermediates.

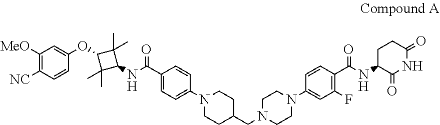 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

### RELATED APPLICATIONS

This application claims priority to, and the benefit of, U.S. Application No. 63/562,672, filed Mar. 7, 2024, which is hereby incorporated by reference in its entirety.

### BACKGROUND

This application relates to a compound that has been shown to be a useful modulator of targeted protein ubiquitination and degradation via the ubiquitin-proteasome system. In particular, the application relates to processes for manufacturing the compound. The application further relates to crystalline forms, amorphous forms, and stable forms of the compound.

### SUMMARY

In one aspect, this application pertains to a method Step (III):
reacting Intermediate I-4:

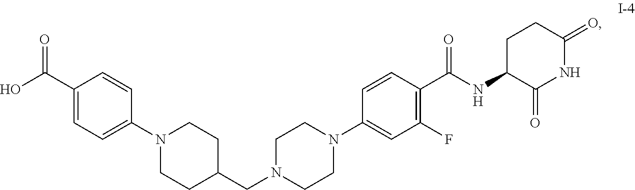 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

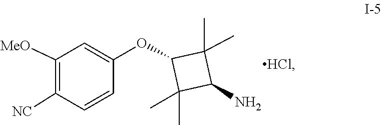 [SMILES: COc1cc(O[C@H]2C(C)(C)[C@H](N)C2(C)C)ccc1C#N]

in a reaction mixture, wherein the reaction mixture comprises a base, a coupling reagent, and an additive in a solvent to provide Compound A:

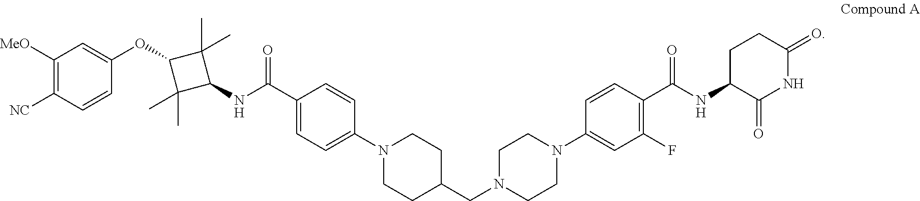 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Compound A is provided in crude form.

In some embodiments, Compound A is provided in crystalline form.

In some embodiments, the salt of Intermediate I-4 is a hydrochloride salt, optionally a bis-hydrochloride salt.

In some embodiments, the base of Step (III) is an amine base.

In some embodiments, the molar ratio of the base of Step (III) to Intermediate I-4 is about 1:1 to about 10:1.

In some embodiments, the molar ratio of the coupling reagent of Step (III) to Intermediate I-4 is about 1:1 to about 2:1.

In some embodiments, the additive of Step (III) is a racemization inhibitor.

In some embodiments, the additive of Step (III) is ethyl 2-(hydroxyimino) cyanoacetate (OXYMA) or 2-hydroxypyridine 1-oxide (HOPO).

In some embodiments, the molar ratio of the additive of Step (III) to Intermediate I-4 is about 0.1:1 to about 2:1.

In some embodiments, the solvent of Step (III) is a polar solvent.

In some embodiments, Step (III) is conducted at a temperature of about −30° C. to about 30° C., or about −10° C. to about 10° C.

In some embodiments, the method further comprises purifying crude Compound A to provide purified Compound A.

In some embodiments, the method provides purified, amorphous Compound A.

In some embodiments, the method further comprises Step (V):
(V) crystallizing Compound A to provide purified crystalline Compound A.

In one aspect, this application pertains to a method comprising Step (I):
reductively aminating Intermediate I-1:

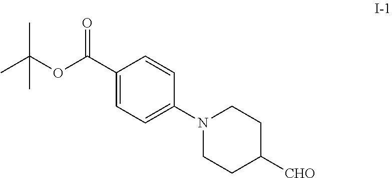 [SMILES: CC(C)(C)OC(=O)c1ccc(N2CCC(C=O)CC2)cc1]

with Intermediate I-2:

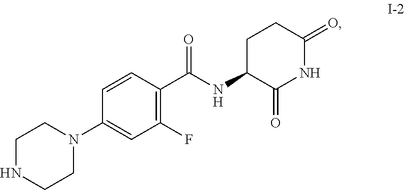 [SMILES: O=C1CC[C@H](NC(=O)c2ccc(N3CCNCC3)cc2F)C(=O)N1]

or a salt thereof,
in a reaction mixture, wherein the reaction mixture comprises a base, a reducing agent, and a solvent to provide wet Intermediate I-3:

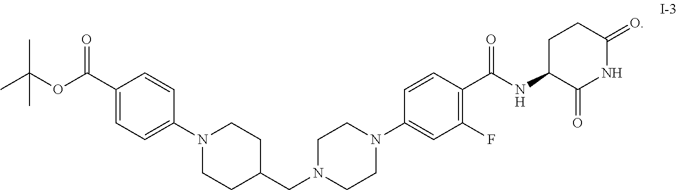 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In some embodiments, the salt of Intermediate I-2 is a hydrochloride salt.

In some embodiments, the base of Step (I) is an amine base or a carbonate salt.

In some embodiments, the molar ratio of the base of Step (I) to Intermediate I-1 is about 1:1 to about 6:1.

In some embodiments, the molar ratio of the reducing agent of Step (I) to Intermediate I-1 is about 1:1 to about 3:1, optionally about 1:1 to about 2:1.

In some embodiments, the solvent of Step (I) is a polar solvent.

In some embodiments, Step (I) is conducted at a temperature of about −30° C. to about 30° C.; optionally about −10° C. to about 10° C.

In one aspect, this application pertains to a method comprising Step (II): reacting Intermediate I-3:

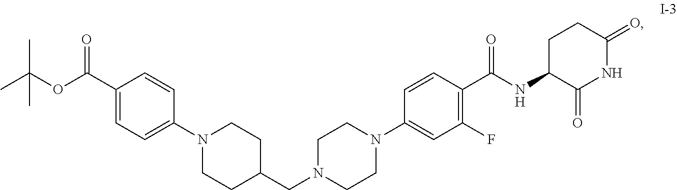 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid
in a reaction mixture to provide Intermediate I-4:

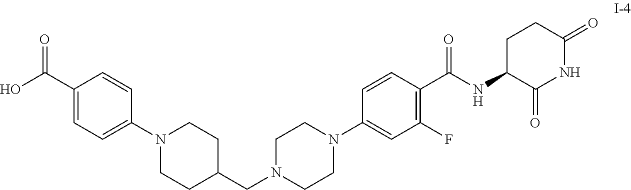 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, wherein the reaction mixture comprises a first solvent.

In some embodiments, Step (II) provides a hydrochloride salt of Intermediate I-4, optionally a bis-hydrochloride salt of Intermediate I-4.

In some embodiments, the acid of Step (II) is an organic acid.

In some embodiments, the first solvent of Step (II) is dichloromethane.

In some embodiments, Step (II) is conducted at a temperature of about 20° C. to about 50° C.

In some embodiments, Intermediate I-4 is at least 95%, at least 96%, at least 97%, at least 98%, or at least 99% pure, as measured by HPLC.

In some embodiments, Intermediate I-4:

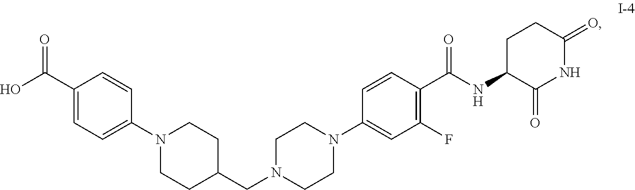 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, is prepared by a method comprising reacting Intermediate I-3:

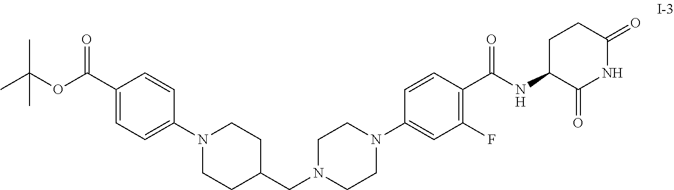 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid in a reaction mixture comprising a first solvent.

In some embodiments, Intermediate I-3 is prepared by a method comprising reductively aminating Intermediate I-1:

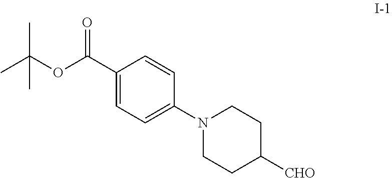 [SMILES: CC(C)(C)OC(=O)c1ccc(N2CCC(C=O)CC2)cc1]

with Intermediate I-2:

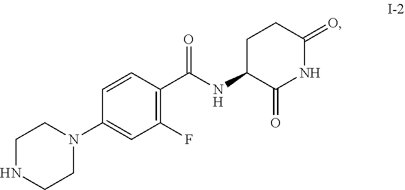 [SMILES: O=C1CC[C@H](NC(=O)c2ccc(N3CCNCC3)cc2F)C(=O)N1]

or a salt thereof,
in a reaction mixture comprising a base, a reducing agent, and a solvent to provide wet

### Intermediate I-3:

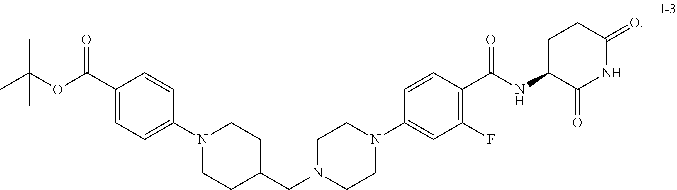 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In one aspect, this application pertains to a method for preparing Compound A, wherein the method comprises:
(a) reacting Intermediate I-1:

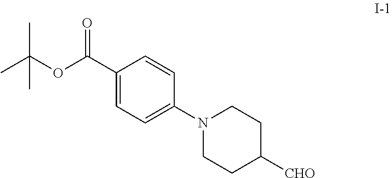 [SMILES: CC(C)(C)OC(=O)c1ccc(N2CCC(C=O)CC2)cc1]

with Intermediate I-2:

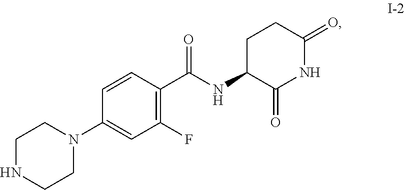 [SMILES: O=C1CC[C@H](NC(=O)c2ccc(N3CCNCC3)cc2F)C(=O)N1]

or a salt thereof,
in a first reaction mixture comprising a base, a reducing agent, and a first solvent to provide wet Intermediate I-3:

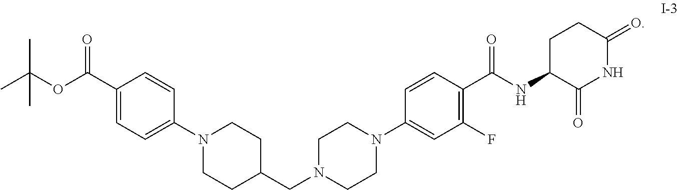 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

(b) drying wet Intermediate I-3;
(c) reacting dried Intermediate I-3:

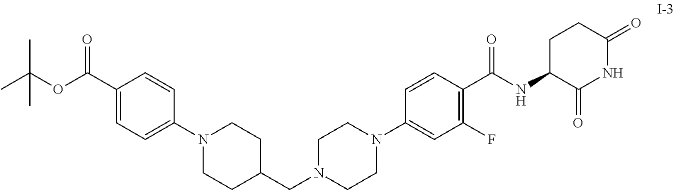 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof,
with an acid in a second reaction mixture to provide Intermediate I-4:

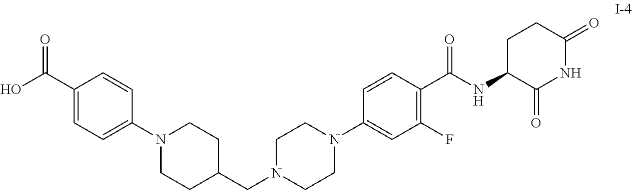 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
wherein the second reaction mixture comprises a second solvent;
(d) reacting Intermediate I-4

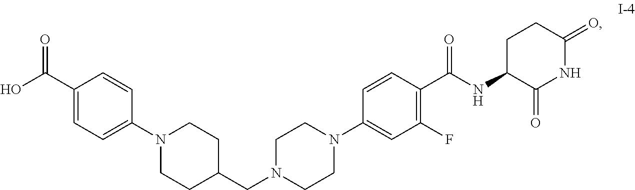 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

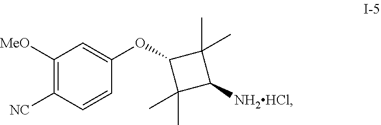

in a third reaction mixture, wherein the third reaction mixture comprises a base, a coupling reagent, and an additive in a third solvent to provide crude Compound A:

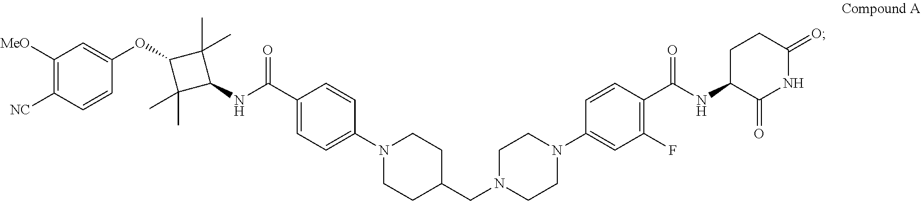 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and, optionally, wherein crude Compound A is purified by recrystallization.

In one aspect, this application pertains to a preparation of 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide (Compound A),

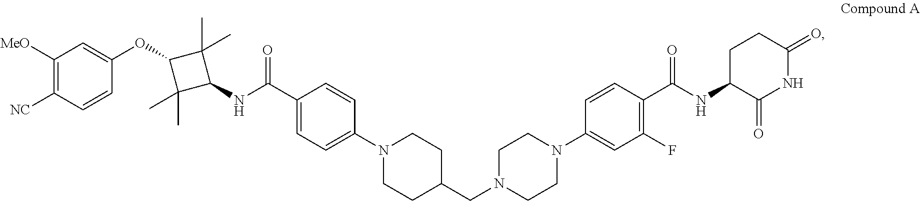 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein Compound A in the preparation has a purity of greater than about 95%, greater than about 96%, greater than about 97%, or greater than about 98%, as measured by HPLC.

In one aspect, this application pertains to a preparation of Compound A, wherein Compound A in the preparation has a purity of greater than about 98%, and comprises less than about 0.2%, less than about 0.3%, less than about 0.4%, or less than about 0.5% of impurity Intermediate I-3:

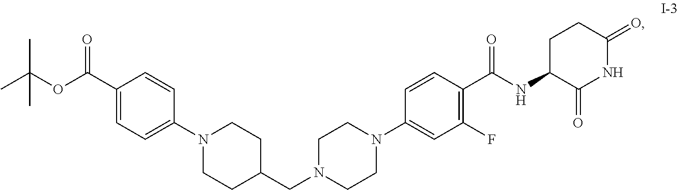 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

as measured by HPLC.

In one aspect, this application pertains to a preparation of Compound A, wherein Compound A in the preparation has a purity of greater than about 98%, and comprises less than about 0.2%, less than about 0.3%, less than about 0.4%, or less than about 0.5% of impurity Intermediate I-4:

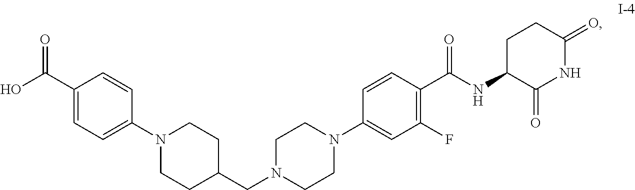 [SMILES: O=C1CC[C@H](NC(=O)c2ccc(N3CCN(CC4CCN(c5ccc(C(=O)O)cc5)CC4)CC3)cc2F)C(=O)N1]

as measured by HPLC.

In one aspect, this application pertains to a preparation of Compound A, wherein Compound A in the preparation has a purity of greater than about 98%, and comprises less than about 0.1%, less than about 0.2%, less than about 0.3%, less than about 0.4%, or less than about 0.5% of:

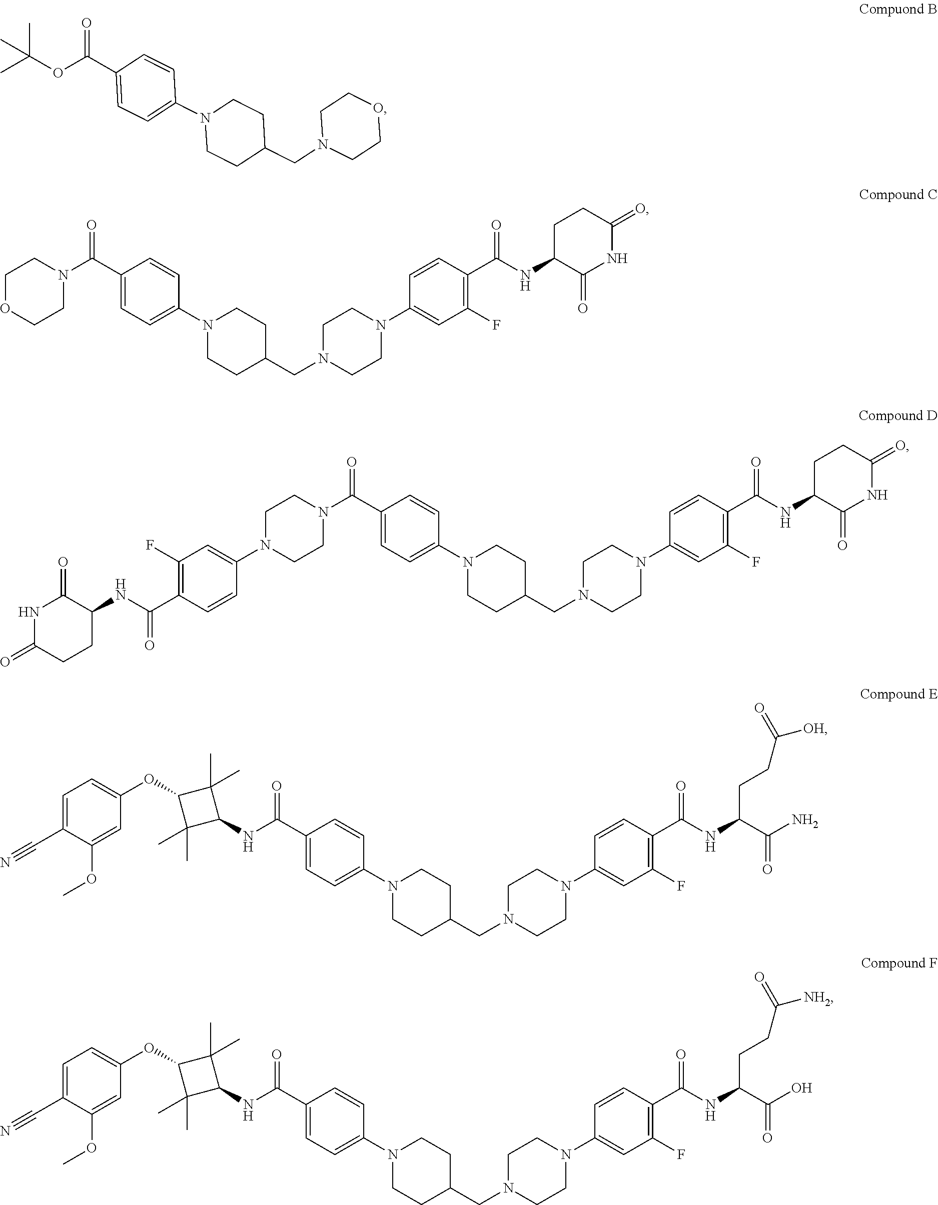 [SMILES: CC(C)(C)OC(=O)c1ccc(N2CCC(CN3CCOCC3)CC2)cc1 ; O=C1CC[C@H](NC(=O)c2ccc(N3CCN(CC4CCN(c5ccc(C(=O)N6CCOCC6)cc5)CC4)CC3)cc2F)C(=O)N1 ; O=C1CC[C@H](NC(=O)c2ccc(N3CCN(CC4CCN(c5ccc(C(=O)N6CCN(c7ccc(C(=O)N[C@H]8CCC(=O)NC8=O)c(F)c7)CC6)cc5)CC4)CC3)cc2F)C(=O)N1 ; COc1cc(O[C@H]2C(C)(C)[C@H](NC(=O)c3ccc(N4CCC(CN5CCN(c6ccc(C(=O)N[C@@H](CCC(=O)O)C(N)=O)c(F)c6)CC5)CC4)cc3)C2(C)C)ccc1C#N ; COc1cc(O[C@H]2C(C)(C)[C@H](NC(=O)c3ccc(N4CCC(CN5CCN(c6ccc(C(=O)N[C@@H](CCC(N)=O)C(=O)O)c(F)c6)CC5)CC4)cc3)C2(C)C)ccc1C#N]

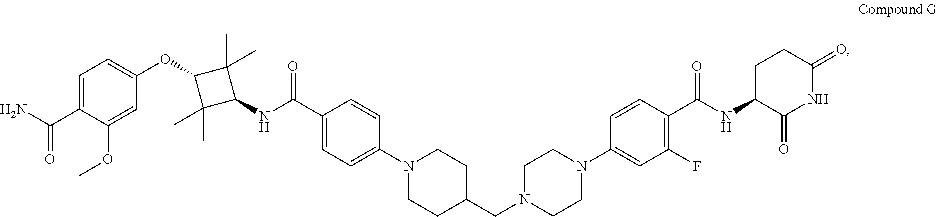 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C(N)=O]

or a combination thereof, as measured by HPLC.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIG. 1 is a flowchart that summarizes the processes for manufacturing Compound A disclosed herein.

### DETAILED DESCRIPTION

In one aspect, the present disclosure is directed to methods for preparing Compound A.

In one aspect, the present disclosure is directed to a method comprising Step (III′):

(III′) reacting Intermediate I-4:

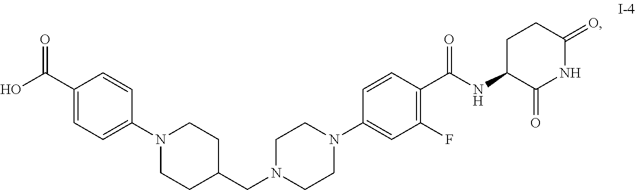 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

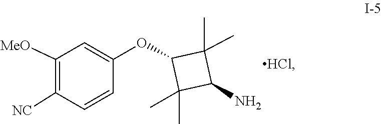 [SMILES: COc1cc(O[C@H]2C(C)(C)[C@H](N)C2(C)C)ccc1C#N]

in a reaction mixture, wherein the reaction mixture comprises a base, a coupling reagent, and an additive in a solvent to provide Compound A:

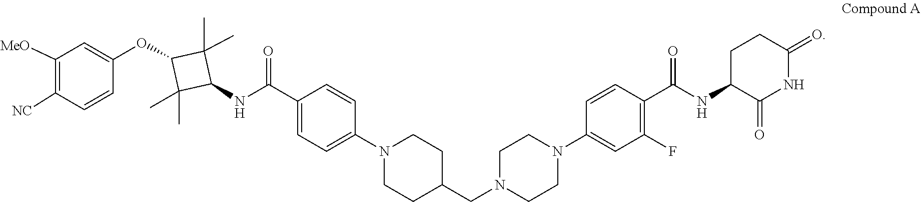 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Step (III′) provides Compound A in crude form.

In some embodiments, Step (III′) followed by Step (IV) provides amorphous Compound A.

In some embodiments, Step (III′) followed by Step (IV) and then Step (V) provides crystalline Compound A.

In some embodiments, Intermediate I-4 in Step (III′) is a hydrochloride salt.

In some embodiments, Intermediate I-4 in Step (III′) is a bis-hydrochloride salt.

In some embodiments, the base of Step (III′) is an amine base.

In some embodiments, the base of Step (III′) is N-methylmorpholine, trimethylamine, triethylamine, N,N-diisopropylethylamine, or N,N-dimethylaniline.

In some embodiments, the molar ratio of the base of Step (III′) to Intermediate I-4 is about 1:1 to about 10:1.

In some embodiments, the molar ratio of the base of Step (III′) to Intermediate I-4 is about 3:1 to about 8:1, or preferably about 4.5:1 to about 6.5:1.

In some embodiments, the molar ratio of the base of Step (III′) to Intermediate I-4 is about 1.0:1, about 1.5:1, about 2.0:1, about 2.5:1, about 3.0:1, about 3.5:1, about 4.0:1, about 4.5:1, about 5.0:1, about 5.5:1, about 6.0:1, about 6.5:1, about 7.0:1, about 7.5:1, about 8.0:1, about 8.5:1, about 9.0:1, about 9.5:1, about 10.0:1.

In some embodiments, the coupling reagent of Step (III′) is a carbodiimide.

In some embodiments, the coupling reagent of Step (III′) is 1-Ethyl-3-(3′-dimethylaminopropyl) carbodiimide or a salt thereof.

In some embodiments, the coupling reagent of Step (III′) is 1-Ethyl-3-(3′-dimethylaminopropyl) carbodiimide hydrochloride.

In some embodiments, the molar ratio of the coupling reagent of Step (III′) to Intermediate I-4 is about 1:1 to about 2:1.

In some embodiments, the molar ratio of the coupling reagent of Step (III′) to Intermediate I-4 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

In some embodiments, the additive of Step (III′) is a racemization inhibitor.

In some embodiments, the racemization inhibitor prevents or reduces the racemization of a chiral center. For example, the racemization of a nitrogen-carbon bond, wherein the carbon is a member of a glutaramide ring, e.g., in Compound A, or a salt thereof.

In some embodiments, the additive of Step (III′) is ethyl 2-(hydroxyimino) cyanoacetate (OXYMA), 2-hydroxypyridine 1-oxide (HOPO), hexafluorophosphate azabenzotriazole tetramethyl uronium (HATU), or hydroxybenzotriazole (HOBt).

In some embodiments, the additive of Step (III′) is ethyl 2-(hydroxyimino) cyanoacetate (OXYMA) or 2-hydroxypyridine 1-oxide (HOPO).

In some embodiments, the additive of Step (III′) is ethyl 2-(hydroxyimino) cyanoacetate (OXYMA).

In some embodiments, the molar ratio of the additive of Step (III′) to Intermediate I-4 is about 0.1:1 to about 2:1.

In some embodiments, the molar ratio of the additive of Step (III′) to Intermediate I-4 is about 0.75:1, about 0.80:1, about 0.85:1, about 0.90:1, about 0.95:1, about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

In some embodiments, the solvent of Step (III′) is a polar solvent.

In some embodiments, the solvent of Step (III′) is dimethylacetamide, dimethylsulfoxide, N-methyl-2-pyrrolidone, 2-methyltetrahydrofuran, dichloromethane, or any combination thereof.

In some embodiments, the solvent of Step (III′) is dimethylacetamide.

In some embodiments, the solvent of Step (III′) is dimethylsulfoxide.

In some embodiments, the solvent of Step (III′) is N-methyl-2-pyrrolidone.

In some embodiments, the solvent of Step (III′) is 2-methyltetrahydrofuran.

In some embodiments, the solvent of Step (III′) is dichloromethane.

In some embodiments, Step (III′) is conducted at a temperature of about −30° C. to about 30° C., or about −10° C. to about 10° C.

In some embodiments, Step (III′) is conducted at a temperature of about −30° C., about −25° C., about −20° C., about −15° C., about −10° C., about −5° C., about 0° C., about 5° C., about 10° C., about 15° C., about 20° C., about 25° C., or about 30° C.

In some embodiments, Step (III′) further comprises Steps (III-i′), (III-ii′), and (III-iii′): (III-i′) adding water to the reaction mixture; (III-ii′) warming the reaction mixture to an elevated temperature, and (III-iii′) agitating the reaction mixture.

In some embodiments, the elevated temperature of Step (III-ii′) is about 10° C. to about 30° C.

In some embodiments, the elevated temperature of Step (III-ii′) is about 10° C., about 15° C., about 20° C., about 25° C., or about 30° C.

In some embodiments, Step (III′) further comprises Step (III-iv′): (III-iv′) filtering the mixture and washing the filtrate with water, methyl tert-butyl ether, acetonitrile, or a combination thereof.

In some embodiments, the filtrate of Step (III-iv′) is washed with a combination of water and methyl tert-butyl ether, optionally wherein the ratio of water to methyl tert-butyl ether in the combination is about 1:1.0 (v/v), about 1:1.1 (v/v), about 1:1.2 (v/v), about 1:1.3 (v/v), about 1:1.4 (v/v), or about 1:1.5 (v/v).

In one aspect, the present disclosure is directed to a method comprising Step (III″):
(III″) reacting Intermediate I-4:

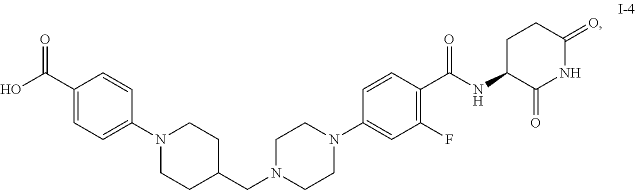 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

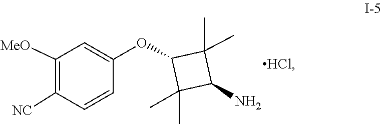 [SMILES: COc1cc(O[C@H]2C(C)(C)[C@H](N)C2(C)C)ccc1C#N]

in a reaction mixture, wherein the reaction mixture comprises a base, a coupling reagent, and an additive in a solvent to provide Compound A:

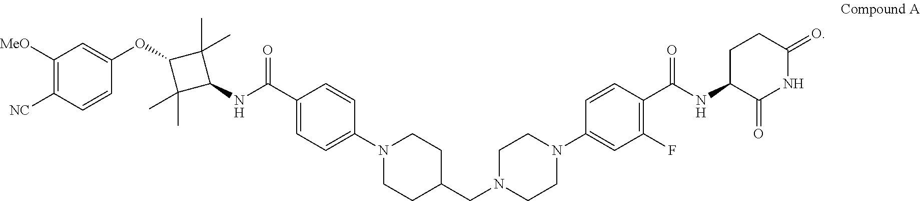 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Step (III″) provides Compound A in crude form.

In some embodiments, Intermediate I-4 in Step (III″) is a hydrochloride salt.

In some embodiments, the salt of Intermediate I-4 is a bis-hydrochloride salt.

In some embodiments, the base of Step (III″) is an amine base.

In some embodiments, the base of Step (III″) is N-methylmorpholine, trimethylamine, triethylamine, N,N-diisopropylethylamine, or N,N-dimethylaniline.

In some embodiments, the molar ratio of the base of Step (III″) to Intermediate I-4 is about 1:1 to about 10:1.

In some embodiments, the molar ratio of the base of Step (III″) to Intermediate I-4 is about 3:1 to about 8:1, or preferably about 4.5:1 to about 6.5:1.

In some embodiments, the molar ratio of the base of Step (III″) to Intermediate I-4 is about 1.0:1, about 1.5:1, about 2.0:1, about 2.5:1, about 3.0:1, about 3.5:1, about 4.0:1, about 4.5:1, about 5.0:1, about 5.5:1, about 6.0:1, about 6.5:1, about 7.0:1, about 7.5:1, about 8.0:1, about 8.5:1, about 9.0:1, about 9.5:1, about 10.0:1.

In some embodiments, the coupling reagent of Step (III″) is a carbodiimide.

In some embodiments, the coupling reagent of Step (III″) is 1-Ethyl-3-(3′-dimethylaminopropyl) carbodiimide or a salt thereof.

In some embodiments, the coupling reagent of Step (III″) is 1-Ethyl-3-(3′-dimethylaminopropyl) carbodiimide hydrochloride.

In some embodiments, the molar ratio of the coupling reagent of Step (III″) to Intermediate I-4 is about 1:1 to about 2:1.

In some embodiments, the molar ratio of the coupling reagent of Step (III″) to Intermediate I-4 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

In some embodiments, the additive of Step (III″) is ethyl 2-(hydroxyimino) cyanoacetate (OXYMA), 2-hydroxypyridine 1-oxide (HOPO), hexafluorophosphate azabenzotriazole tetramethyl uronium (HATU), or hydroxybenzotriazole (HOBt).

In some embodiments, the additive of Step (III″) is ethyl 2-(hydroxyimino) cyanoacetate (OXYMA) or 2-hydroxypyridine 1-oxide (HOPO).

In some embodiments, the additive of Step (III″) is 2-hydroxypyridine 1-oxide (HOPO).

In some embodiments, the additive of Step (III″) is a racemization inhibitor.

In some embodiments, the racemization inhibitor prevents or reduces the racemization of a chiral center. For example, the racemization of a nitrogen-carbon bond, wherein the carbon is a member of a glutaramide ring, e.g., in Compound A, or a salt thereof.

In some embodiments, the molar ratio of the additive of Step (III″) to Intermediate I-4 is about 0.1:1 to about 0.5:1.

In some embodiments, the molar ratio of the additive of Step (III″) to Intermediate I-4 is about 0.10:1, about 0.15:1, about 0.2:1, about 0.25:1, about 0.30:1, about 0.35:1, about 0.40:1, about 0.45:1, or about 0.50:1.

In some embodiments, the molar ratio of the additive of Step (III″) to Intermediate I-4 is about 0.3:1.

In some embodiments, the solvent of Step (III″) is a polar solvent.

In some embodiments, the solvent of Step (III″) is dimethylacetamide, dimethylsulfoxide, N-methyl-2-pyrrolidone, 2-methyltetrahydrofuran, dichloromethane, or any combination thereof.

In some embodiments, the solvent of Step (III″) is dimethylacetamide.

In some embodiments, the solvent of Step (III″) is dimethylsulfoxide.

In some embodiments, the solvent of Step (III″) is N-methyl-2-pyrrolidone.

In some embodiments, the solvent of Step (III″) is 2-methyltetrahydrofuran.

In some embodiments, the solvent of Step (III″) is dichloromethane.

In some embodiments, Step (III″) is conducted at a temperature of about −30° C. to about 30° C., or about −10° C. to about 10° C.

In some embodiments, Step (III″) is conducted at a temperature of about −30° C., about-25° C., about −20° C., about −15° C., about −10° C., about −5° C., about 0° C., about 5° C., about 10° C., about 15° C., about 20° C., about 25° C., or about 30° C.

In some embodiments, Step (III″) further comprises Steps (III-ic″), (III-iic″), (III-iiic″), (III-ivc″), (III-vc″), (III-vic″), (III-viic″), and (III-viiic″):
(III-ic″) adding water to the reaction mixture;
(III-iic″) adding an agent that induces nucleation to the reaction mixture of Step (III-ic″);
(III-iiic″) warming the reaction mixture of Step (III-iic″) to an elevated temperature;
(III-ivc″) agitating the reaction mixture of Step (III-iiic″);
(III-vc″) adding a second solvent to the reaction mixture of Step (III-ivc″);
(III-vic″) agitating the reaction mixture of Step (III-vc″);
(III-viic″) filtering the reaction mixture of Step (III-vic″) to provide a cake and rinsing the cake with water; and
(III-viiic″) filtering and rinsing the cake of Step (III-viic″) with a third solvent.

In some embodiments, the agent that induces nucleation of Step (III-iicc″) is a crystallization promoter. In some embodiments, the agent that induces nucleation of Step (III-iicc″) is a seed crystal of Compound A.

In some embodiments, the elevated temperature of Step (III-iiic″) is about 10° C. to about 35° C.

In some embodiments, the elevated temperature of Step (III-iiic″) is about 10° C., about 15° C., about 20° C., about 25° C., about 30° C., or about 35° C.

In some embodiments, the second solvent of Step (III-vc″) is methyl tert-butyl ether.

In some embodiments, the third solvent of Step (III-viiic″) is methyl tert-butyl ether.

In some embodiments, crude Compound A is further purified to provide purified Compound A.

In some embodiments, crude Compound A is further purified to provide purified, amorphous Compound A.

In some embodiments, crude Compound A is further purified to provide purified, crystalline Compound A.

In some embodiments, purifying crude Compound A comprises Step (IV), which comprises Steps (IV-i), (IV-ii), (IV-iii), and (IV-iv):
(IV-i) combining crude Compound A and a solvent to prepare a mixture;
(IV-ii) heating the mixture;
(IV-iii) agitating the mixture; and
(IV-iv) filtering the mixture to provide purified Compound A; optionally wherein steps (IV-i), (IV-ii), (IV-iii), and/or (IV-iv) are repeated.

In some embodiments, the solvent of Step (IV-i) is water, acetonitrile, or a combination thereof.

In some embodiments, the ratio of water to acetonitrile in the combination used in Step (IV-i) is about 0.5:1 (v/v), about 0.6:1 (v/v), about 0.7:1 (v/v), about 0.8:1 (v/v), about 0.9:1 (v/v), about 1.0:1 (v/v), about 1.1:1 (v/v), about 1.2:1 (v/v), about 1.3:1 (v/v), about 1.4:1 (v/v), or about 1.5:1 (v/v).

In some embodiments, the mixture of Step (IV-ii) is heated to a temperature of about 20° C. to about 50° C.

In some embodiments, the mixture of Step (IV-ii) is heated to a temperature of about 20° C., about 25° C., about 30° C., about 35° C., about 40° C., about 45° C., or about 50° C.

In some embodiments, the mixture of Step (IV-ii) is heated to a temperature of about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In some embodiments, the purified Compound A of Step (IV-iv) is washed with a combination of water and acetonitrile.

In some embodiments, the ratio of water to acetonitrile in the combination used in Step (IV-iv) is about 0.5:1 (v/v), about 0.6:1 (v/v), about 0.7:1 (v/v), about 0.8:1 (v/v), about 0.9:1 (v/v), about 1.0:1 (v/v), about 1.1:1 (v/v), about 1.2:1 (v/v), about 1.3:1 (v/v), about 1.4:1 (v/v), or about 1.5:1 (v/v).

In some embodiments, the method further comprises Step (V):
(V) crystallizing Compound A to provide purified crystalline Compound A.

In some embodiments, Step (V) comprises Steps (V-i), (V-ii), (V-iii), (V-iv), (V-v), and (V-vi):
(V-i) combining a first solvent and amorphous Compound A to prepare a mixture;
(V-ii) heating the mixture of Step (V-i);
(V-iii) adding a second solvent to the mixture of Step (V-ii);
(V-iv) adding an agent that induces nucleation to the mixture of Step (V-iii);
(V-v) reducing the volume of the mixture of Step (V-iv); and
(V-vi) agitating the mixture of Step (V-iv).

In some embodiments, the first solvent of Step (V-i) is dichloromethane.

In some embodiments, the mixture of Step (V-ii) is heated to a temperature of about 20° C. to about 50° C.

In some embodiments, the mixture of Step (V-ii) is heated to a temperature of about 20° C., about 25° C., about 30° C., about 35° C., about 40° C., about 45° C., or about 50° C.

In some embodiments, the second solvent of Step (V-iii) is an alcohol; optionally wherein the second solvent of Step (V-iii) is methanol or ethanol.

In some embodiments, the agent that induces nucleation of Step (V-iv) is a crystallization promoter.

In some embodiments, the crystallization promoter of Step (V-iv) is a seed crystal of Compound A.

In some embodiments, Step (V) further comprises Steps (V-vii), (V-viii), (V-ix), (V-x), (V-xi), and (V-xii):
(V-vii) adding an additional amount of the second solvent of Step (V-iii) to the mixture of Step (V-vi) to provide a second mixture;
(V-viii) removing the first solvent of Step (V-i) from the second mixture of Step (V-vii) to provide a concentrated second mixture;
(V-ix) agitating the concentrated second mixture of Step (V-viii);
(V-x) cooling the concentrated second mixture of Step (V-ix) to provide a slurry; (V-xi) filtering the slurry of Step (V-x) to provide a cake; and
(V-xii) washing the cake of Step (V-xi) with the second solvent to provide purified crystalline Compound A.

In some embodiments, the first solvent of Step (V-viii) is removed by distillation.

In some embodiments, the first solvent of Step (V-viii) is removed by vacuum distillation.

In some embodiments, the first solvent of Step (V-viii) is removed by atmospheric distillation.

In some embodiments, the purified crystalline Compound A prepared in Step (V-xii) is at least 95%, at least 96%, at least 97%, at least 98%, or at least 99% pure.

In some embodiments, the purified crystalline Compound A prepared in Step (V-xii) is at least 95%, at least 96%, at least 97%, at least 98%, or at least 99% pure as measured by HPLC.

In one aspect, the present disclosure is directed to a method comprising Step (I′):
(I) reductively aminating Intermediate I-1:

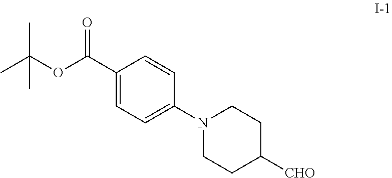 [SMILES: CC(C)(C)OC(=O)c1ccc(N2CCC(C=O)CC2)cc1]

with Intermediate I-2:

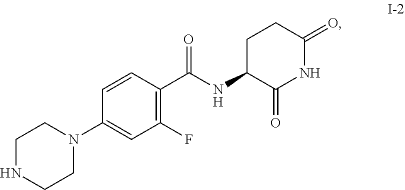 [SMILES: O=C1CC[C@H](NC(=O)c2ccc(N3CCNCC3)cc2F)C(=O)N1]

or a salt thereof,
in a reaction mixture, wherein the reaction mixture comprises a base, a reducing agent, and a solvent to provide wet Intermediate I-3:

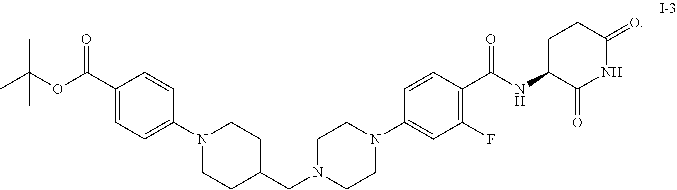 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In some embodiments, the salt of Intermediate I-2 is a hydrochloride salt.

In some embodiments, the base of Step (I′) is an amine base or a carbonate salt.

In some embodiments, the base of Step (I′) is N-methylmorpholine, trimethylamine, triethylamine, N,N-diisopropylethylamine, N,N-dimethylaniline, lithium carbonate, sodium carbonate, potassium carbonate, or magnesium carbonate.

In some embodiments, the molar ratio of the base of Step (I′) to Intermediate I-1 is about 1:1 to about 6:1.

In some embodiments, the molar ratio of the base of Step (I′) to Intermediate I-1 is about 1.0:1, about 1.5:1, about 2.0:1, about 2.5:1, about 3.0:1, about 3.5:1, about 4.0:1, about 4.5:1, about 5.0:1, about 5.5:1, or about 6.0:1.

In some embodiments, the molar ratio of the base of Step (I′) to Intermediate I-1 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, about 2.00:1, about 2.05:1, about 2.10:1, about 2:15:1, about 2:20:1, about 2.25:1, about 2.30:1, about 2.35:1, about 2.40:1, about 2.45:1, about 2.50:1, about 2.55:1, about 2.60:1, about 2.65:1, about 2.70:1, about 2.75:1, about 2.80:1, about 2.85:1, about 2.90:1, about 2.95:1, about 3.00:1, about 3.05:1, about 3.10:1, about 3.15:1, about 3.20:1, about 3.25:1, about 3.30:1, about 3.35:1, about 3.40:1, about 3.45:1, about 3.50:1, about 3.55:1, about 3.60:1, about 3.65:1, about 3.70:1, about 3.75:1, about 3.80:1, about 3.85:1, about 3.90:1, about 3.95:1, or about 4.00:1.

In some embodiments, the reducing agent of Step (I′) is sodium triacetoxyborohydride, sodium borohydride, or sodium cyanoborohydride.

In some embodiments, the molar ratio of the reducing agent of Step (I′) to Intermediate I-1 is about 1:1 to about 3:1, optionally about 1:1 to about 2:1.

In some embodiments, the molar ratio of the reducing agent of Step (I′) to Intermediate I-1 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, about 2.00:1, about 2.05:1, or about 2.10:1.

In some embodiments, the solvent of Step (I′) is a polar solvent.

In some embodiments, the solvent of Step (I′) is dimethylacetamide, dimethylsulfoxide, N-methyl-2-pyrrolidone, 2-methyltetrahydrofuran, tetrahydrofuran, 1,4-dioxane, dichloromethane, dimethylformamide, or any combination thereof.

In some embodiments, the solvent of Step (I′) is dimethylacetamide.

In some embodiments, the solvent of Step (I′) is N-methyl-2-pyrrolidone.

In some embodiments, the solvent of Step (I′) is 2-methyltetrahydrofuran.

In some embodiments, Step (I′) is conducted at a temperature of about −30° C. to about 30° C.; optionally about −10° C. to about 10° C.

In some embodiments, Step (I′) is conducted at a temperature of about −30° C., about-25° C., about −20° C., about −15° C., about −10° C., about −5° C., about 0° C., about 5° C., about 10° C., about 15° C., about 20° C., about 25° C., or about 30° C.

In one aspect, the present disclosure is directed to a method comprising Step (I″):
(I″) reductively aminating Intermediate I-1:

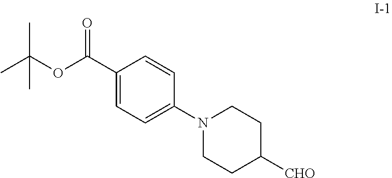 [SMILES: CC(C)(C)OC(=O)c1ccc(N2CCC(C=O)CC2)cc1]

with Intermediate I-2:

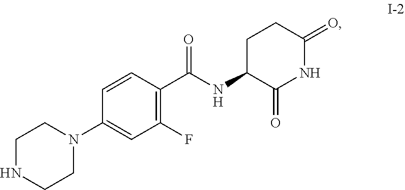 [SMILES: O=C1CC[C@H](NC(=O)c2ccc(N3CCNCC3)cc2F)C(=O)N1]

or a salt thereof,
in a reaction mixture, wherein the reaction mixture comprises a base, a reducing agent, and a solvent to provide wet Intermediate I-3:

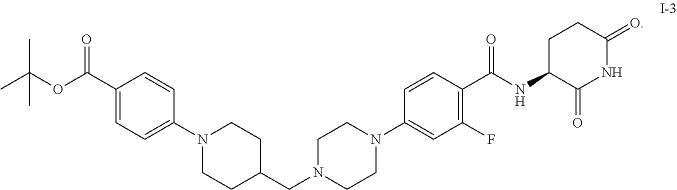 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In some embodiments, the salt of Intermediate I-2 is a hydrochloride salt.

In some embodiments, the base of Step (I″) is an amine base or a carbonate salt.

In some embodiments, the base of Step (I″) is N-methylmorpholine, trimethylamine, triethylamine, N,N-diisopropylethylamine, N,N-dimethylaniline, lithium carbonate, sodium carbonate, potassium carbonate, or magnesium carbonate.

In some embodiments, the molar ratio of the base of Step (I″) to Intermediate I-1 is about 1:1 to about 6:1.

In some embodiments, the molar ratio of the base of Step (I″) to Intermediate I-1 is about 1.0:1, about 1.5:1, about 2.0:1, about 2.5:1, about 3.0:1, about 3.5:1, about 4.0:1, about 4.5:1, about 5.0:1, about 5.5:1, or about 6.0:1.

In some embodiments, the molar ratio of the base of Step (I″) to Intermediate I-1 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, about 2.00:1, about 2.05:1, about 2.10:1, about 2:15:1, about 2:20:1, about 2.25:1, about 2.30:1, about 2.35:1, about 2.40:1, about 2.45:1, about 2.50:1, about 2.55:1, about 2.60:1, about 2.65:1, about 2.70:1, about 2.75:1, about 2.80:1, about 2.85:1, about 2.90:1, about 2.95:1, about 3.00:1, about 3.05:1, about 3.10:1, about 3.15:1, about 3.20:1, about 3.25:1, about 3.30:1, about 3.35:1, about 3.40:1, about 3.45:1, about 3.50:1, about 3.55:1, about 3.60:1, about 3.65:1, about 3.70:1, about 3.75:1, about 3.80:1, about 3.85:1, about 3.90:1, about 3.95:1, or about 4.00:1.

In some embodiments, the reducing agent of Step (I″) is sodium triacetoxyborohydride, sodium borohydride, or sodium cyanoborohydride.

In some embodiments, the molar ratio of the reducing agent of Step (I″) to Intermediate I-1 is about 1:1 to about 3:1, optionally about 1:1 to about 2:1.

In some embodiments, the molar ratio of the reducing agent of Step (I″) to Intermediate I-1 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, about 2.00:1, about 2.05:1, or about 2.10:1.

In some embodiments, the solvent of Step (I″) is a polar solvent.

In some embodiments, the solvent of Step (I″) is dimethylacetamide, acetonitrile, dimethylsulfoxide, N-methyl-2-pyrrolidone, 2-methyltetrahydrofuran, tetrahydrofuran, 1,4-dioxane, dichloromethane, dimethylformamide or any combination thereof.

In some embodiments, the solvent of Step (I″) is dimethylsulfoxide, acetonitrile, and water.

In some embodiments, the solvent of Step (I″) is dimethylsulfoxide.

In some embodiments, the solvent of Step (I″) is acetonitrile.

In some embodiments, the solvent of Step (I″) is dimethylsulfoxide and acetonitrile.

In some embodiments, Step (I″) is conducted at a temperature of about −30° C. to about 30° C.; optionally about −10° C. to about 10° C.

In some embodiments, Step (I″) is conducted at a temperature of about −30° C. to about 30° C.; optionally about 5° C. to about 30° C.

In some embodiments, Step (I″) is conducted at a temperature of about −30° C., about-25° C., about −20° C., about −15° C., about −10° C., about −5° C., about 0° C., about 5° C., about 10° C., about 15° C., about 20° C., about 25° C., or about 30° C.

In some embodiments, Step (I″) further comprises Steps (I-ia″), (I-iia″), (I-iiia″), (I-iva″), (I-va″), (I-via″), and (I-viia″):
(I-ia″) adding a second solvent to the reaction mixture;
(I-iia″) adding a third solvent to the reaction mixture of Step (I-ia″);
(I-iiia″) adding an agent that induces nucleation to the reaction mixture of Step (I-iia″);
(I-iva″) adding water to the reaction mixture of Step (I-iiia″);
(I-va″) cooling the reaction mixture of Step (I-iva″);
(I-via″) agitating the reaction mixture of Step (I-va″); and
(I-viia″) filtering the reaction mixture of Step (I-va″) to provide a cake and rinsing the cake with a fourth solvent.

In some embodiments, the second solvent of Step (I-ia″) is (i) water; or (ii) water and acetonitrile.

In some embodiments, the third solvent of Step (I-iia″) is (i) water; or (ii) water and acetonitrile.

In some embodiments, the agent that induces nucleation of Step (I-iiia″) is a crystallization promoter.

In some embodiments, the crystallization promoter of Step (I-iiia″) is a seed crystal of Intermediate I-3.

In some embodiments, Step (I-va″) is conducted at a temperature of about 5° C. to about 25° C.

In some embodiments, the fourth solvent of Step (I-viia″) is water.

In some embodiments, Step (I-viia″) is repeated one or more times.

In some embodiments, the dried Intermediate I-3 is at least 95%, at least 96%, at least 97%, at least 98%, or at least 99% pure.

In some embodiments, the dried Intermediate I-3 is at least 95%, at least 96%, at least 97%, at least 98%, or at least 99% pure as measured by HPLC.

In one aspect, the present disclosure is directed to a method comprising Step (II′): (II′) reacting Intermediate I-3:

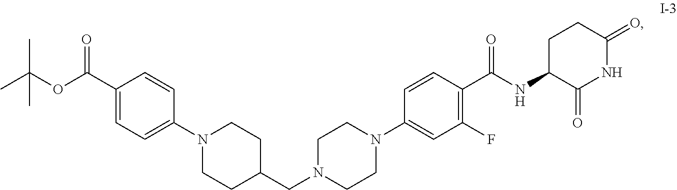 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid in a reaction mixture to provide Intermediate I-4:

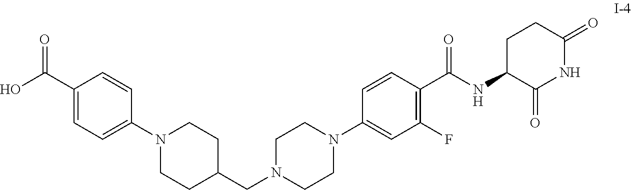 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, wherein the reaction mixture comprises a first solvent.

In some embodiments, Intermediate I-3 is dried prior to Step (II′).

In some embodiments, Step (II′) provides a hydrochloride salt of Intermediate I-4.

In some embodiments, Step (II′) provides a bis-hydrochloride salt of Intermediate I-4.

In some embodiments, the hydrochloride salt of Intermediate I-4 is a monohydrochloride salt.

In some embodiments, the acid of Step (II′) is an organic acid, optionally trifluoroacetic acid.

In some embodiments, the acid of Step (II′) is an inorganic acid, optionally hydrochloric or phosphoric acid.

In some embodiments, the first solvent of Step (II′) is dichloromethane.

In some embodiments, Step (II′) is conducted at a temperature of about 20° C. to about 50° C.

In some embodiments, Step (II′) is conducted at a temperature of about 20° C., about 25° C., about 30° C., about 35° C., about 40° C., about 45° C., or about 50° C.

In some embodiments, Step (II′) further comprises Steps (II-i′), (II-ii′), (II-iii′), (II-iv′), and (II-v′):
(II-i′) adding a second solvent to the reaction mixture of Step (II′);
(II-ii′) distillatively removing the first solvent of Step (II′) and second solvent of Step (II-i′) from the reaction mixture of Step (II-i′);
(II-iii′) adding an additional amount of the second solvent of Step (II-i′) to the reaction mixture of Step (II-ii′);
(II-iv′) adding an aqueous basic solution to the reaction mixture of Step (II-iii′) such that the pH of the reaction mixture is about pH 5.5 to about pH 6.5; and
(II-v′) filtering the reaction mixture of Step (II-iv′) and washing the resulting cake with water to provide wet Intermediate I-4.

In some embodiments, the second solvent of Step (II-i′) is acetonitrile.

In some embodiments, the aqueous basic solution of Step (II-iv′) comprises sodium bicarbonate.

In some embodiments, Step (II′) further comprises Step (II-vi′) and optionally Step (II-vii′):

(II-vi′) filtering and washing the wet Intermediate I-4 with a second solvent; and/or (II-vii′) azeotropically drying wet Intermediate I-4 using a third solvent, to provide dried

### Intermediate I-4.

In some embodiments, the second solvent of Step (II-vi′) is (i) water; or (ii) water and acetonitrile.

In some embodiments, the third solvent of Step (II-vii′) is toluene.

In some embodiments, Step (II′) further comprises Steps (II-ia′), (II-iia′), (II-iiia′), (II-iva′), and (II-va′):
(II-ia′) adding a second solvent to the reaction mixture of Step (II′);
(II-iia′) distillatively removing the first solvent of Step (II′) and the second solvent of Step
(II-ia′) from the reaction mixture of Step (II-ia′);
(II-iiia′) adding an additional amount of the second solvent of Step (II-ia′) to the reaction mixture of Step (II-iia′);
(II-iva′) adding an aqueous acidic solution to the reaction mixture of Step (II-iiia′); and
(II-va′) filtering the reaction mixture of Step (II-iva′) and washing the resulting cake with water to provide wet Intermediate I-4.

In some embodiments, the second solvent of Step (II-ia′) is acetonitrile.

In some embodiments, the aqueous acidic solution of Step (II-iva′) comprises hydrochloric acid.

In some embodiments, Intermediate I-4 is at least 95%, at least 96%, at least 97%, at least 98%, or at least 99% pure.

In some embodiments, Intermediate I-4 is at least 95%, at least 96%, at least 97%, at least 98%, or at least 99% pure as measured by HPLC.

In one aspect, the present disclosure is directed to a method comprising Step (II″):
(II″) reacting dried Intermediate I-3:

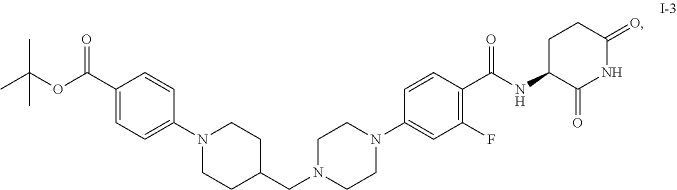 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid in a reaction mixture to provide Intermediate I-4:

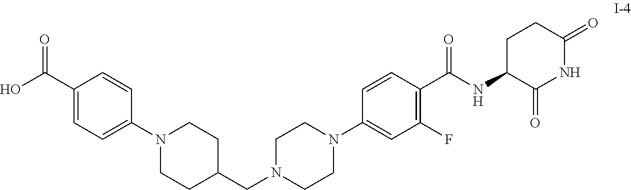 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, wherein the reaction mixture comprises a first solvent.

In some embodiments, Step (II″) provides a hydrochloride salt of Intermediate I-4.

In some embodiments, the hydrochloride salt of Intermediate I-4 is a bis-hydrochloride salt.

In some embodiments, the acid of Step (II″) is an inorganic acid, optionally hydrochloric or phosphoric acid.

In some embodiments, the acid of Step (II″) is an organic acid, optionally trifluoroacetic acid.

In some embodiments, the first solvent of Step (II″) is dichloromethane.

In some embodiments, Step (II″) is conducted at a temperature of about 10° C. to about 50° C.

In some embodiments, Step (II″) is conducted at a temperature of about 20° C., about 25° C., about 30° C., about 35° C., about 40° C., about 45° C., or about 50° C.

In some embodiments, Step (II″) further comprises Steps (II-ib″), (II-iib″), (II-iiib″), (II-ivb″), and (II-vb″):
(II-ib″) adding a second solvent to the reaction mixture of Step (II″);
(II-iib″) adding an agent that induces nucleation to the reaction mixture of Step (II-ib″);)
(II-iiib″) adding an aqueous acidic solution to the reaction mixture of Step (II-iib″); (II-ivb″) agitating the reaction mixture of Step (II-iiib″); and
(II-vb″) filtering the reaction mixture of Step (II-ivb″) and washing the resulting cake with acetonitrile to provide wet Intermediate I-4.

In some embodiments, the second solvent of Step (II-ib″) is acetonitrile.

In some embodiments, the agent that induces nucleation of Step (II-iib″) is a crystallization promoter.

In some embodiments, the crystallization promoter of Step (II-iib″) is Intermediate I-4.

In some embodiments, the aqueous acidic solution of Step (II-iiib″) is about 20%, about 25%, about 30%, about 35%, about 35%, or about 40% (v/v) hydrochloric acid.

In some embodiments, Step (II-ivb″) is conducted at a temperature of about 5° C. to about 30° C.

In some embodiments, Step (II″) provides a bis-hydrochloride salt of Intermediate I-4.

In some embodiments, Intermediate I-4 is at least 95%, at least 96%, at least 97%, at least 98%, or at least 99% pure.

In some embodiments, Intermediate I-4 is at least 95%, at least 96%, at least 97%, at least 98%, or at least 99% pure as measured by HPLC.

In one aspect, the present disclosure is directed to a method comprising Step (III″):

(III′″) reacting Intermediate I-4:

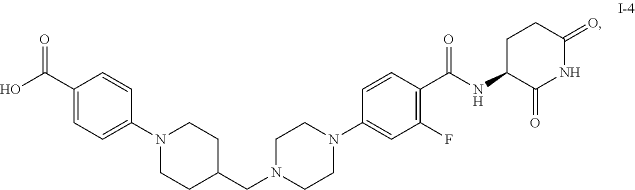 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

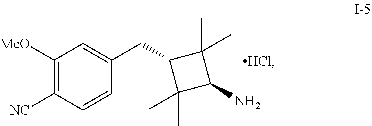 [SMILES: COc1cc(C[C@H]2C(C)(C)[C@H](N)C2(C)C)ccc1C#N]

in a reaction mixture, wherein the reaction mixture comprises a base, a coupling reagent, and an additive in a solvent to provide crystalline Compound A:

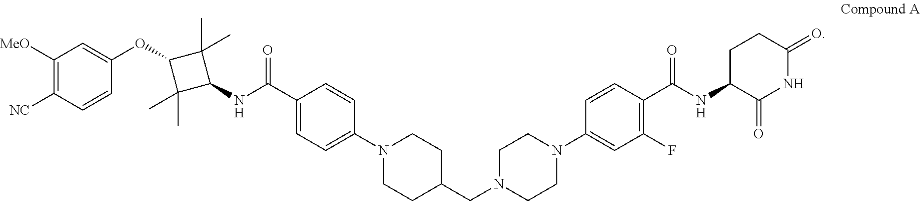 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Step (III′″) provides Compound A in crude form.

In some embodiments, Step (III′″) followed by Step (III-A′″) provides Compound A in crystalline form.

In some embodiments, the salt of Intermediate I-4 is a bis-hydrochloride salt.

In some embodiments, the base of Step (III′″) is an amine base.

In some embodiments, the base of Step (III″) is N-methylmorpholine, trimethylamine, triethylamine, N,N-diisopropylethylamine, or N,N-dimethylaniline.

In some embodiments, the molar ratio of the base of Step (III′″) to Intermediate I-4 is about 1:1 to about 10:1.

In some embodiments, the molar ratio of the base of Step (III″) to Intermediate I-4 is about 3:1 to about 8:1, or preferably about 4.5:1 to about 6.5:1.

In some embodiments, the molar ratio of the base of Step (III′″) to Intermediate I-4 is about 1.0:1, about 1.5:1, about 2.0:1, about 2.5:1, about 3.0:1, about 3.5:1, about 4.0:1, about 4.5:1, about 5.0:1, about 5.5:1, about 6.0:1, about 6.5:1, about 7.0:1, about 7.5:1, about 8.0:1, about 8.5:1, about 9.0:1, about 9.5:1, about 10.0:1.

In some embodiments, the coupling reagent of Step (III′″) is a carbodiimide.

In some embodiments, the coupling reagent of Step (III″) is 1-Ethyl-3-(3′-dimethylaminopropyl) carbodiimide or a salt thereof.

In some embodiments, the coupling reagent of Step (III′″) is 1-Ethyl-3-(3′-dimethylaminopropyl) carbodiimide hydrochloride.

In some embodiments, the molar ratio of the coupling reagent of Step (III″) to Intermediate I-4 is about 1:1 to about 2:1.

In some embodiments, the molar ratio of the coupling reagent of Step (III′″) to Intermediate I-4 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

In some embodiments, the additive of Step (III′″) is ethyl 2-(hydroxyimino) cyanoacetate (OXYMA), 2-hydroxypyridine 1-oxide (HOPO), hexafluorophosphate azabenzotriazole tetramethyl uronium (HATU), or hydroxybenzotriazole (HOBt).

In some embodiments, the additive of Step (III′″) is ethyl 2-(hydroxyimino) cyanoacetate (OXYMA) or 2-hydroxypyridine 1-oxide (HOPO).

In some embodiments, the additive of Step (III″) is ethyl 2-(hydroxyimino) cyanoacetate

(OXYMA).

In some embodiments, the additive of Step (III′″) is 2-hydroxypyridine 1-oxide (HOPO).

In some embodiments, the molar ratio of the additive of Step (III′″) to Intermediate I-4 is about 0.1:1 to about 0.5:1.

In some embodiments, the molar ratio of the additive of Step (III″) to Intermediate I-4 is about 0.10:1, about 0.15:1, about 0.2:1, about 0.25:1, about 0.30:1, about 0.35:1, about 0.40:1, about 0.45:1, about 0.50:1.

In some embodiments, the solvent of Step (III′″) is a polar solvent.

In some embodiments, the solvent of Step (III′″) is dimethylacetamide, dimethylsulfoxide, N-methyl-2-pyrrolidone, 2-methyltetrahydrofuran, dichloromethane, or any combination thereof.

In some embodiments, the solvent of Step (III′″) is dimethylacetamide.

In some embodiments, the solvent of Step (III′″) is dimethylsulfoxide.

In some embodiments, the solvent of Step (III″) is N-methyl-2-pyrrolidone.

In some embodiments, the solvent of Step (III′″) is 2-methyltetrahydrofuran.

In some embodiments, the solvent of Step (III″) is dichloromethane.

In some embodiments, Step (III′″) is conducted at a temperature of about −30° C. to about 30° C., or about −10° C. to about 10° C.

In some embodiments, Step (III″) is conducted at a temperature of about −30° C., about-25° C., about −20° C., about −15° C., about −10° C., about −5° C., about 0° C., about 5° C., about 10° C., about 15° C., about 20° C., about 25° C., or about 30° C.

In some embodiments, Step (III″) further comprises Step (III-A′″).

In some embodiments, Step (III-A′″) further comprises Step (III-Ai′″), Step (III-Aii″′), Step (III-Aiii′″), Step (III-Aiv″), Step (III-Av′″), Step (III-Avi″), Step (III-Avii″″), Step (III-Aviii′″), Step (III-Aix′″), Step (III-Ax″), Step (III-Axi′″), Step (III-Axii′″), Step (III-Axiii′″), Step (III-Axiv′″), Step (III-Axv″), Step (III-Axvi′″), Step (III-Axvii′″), and Step (III-Axviii″″):
(III-Ai′″) adding the reaction mixture comprising crude Compound A to a second reaction mixture comprising water and an agent that induces nucleation to provide a third reaction mixture;
(III-Aii″) adding methyl tert-butyl ether to the third reaction mixture of Step (III-Ai “″);
(III-Aiii”′) agitating the third reaction mixture of Step (III-Aii″″) at a temperature of about 15-25° C.;
(III-Aiv′″) stopping the agitating of the third reaction mixture of Step (III-Aiii″) and allowing the phases to separate into layers;
(III-Av″) removing the bottom layer from the third reaction mixture in Step (III-Aiv″) and filtering the solids followed by rinsing, and drying to provide wet, crude Compound A. (III-Avi′″) charging a separate crystallization reactor with ethanol;
(III-Avii′″) heating the crystallization reactor of Step (III-Avi′″) at a temperature of about 15-25° C.;
(III-Aviii′″) adding dichloromethane and wet, crude Compound A in a separate vessel to provide a fourth reaction mixture;
(III-Aix′″) stirring the fourth reaction mixture of Step (III-Aviii″″);
(III-Ax′″) stopping the stirring of the fourth reaction of Step (III-Aix″) and allowing the phases to separate into layers;
(III-Axi′″) transferring the bottom layer of the fourth reaction mixture of Step (III-Ax″″) to the crystallization reactor of Step (III-Avii″);
(III-Axii″″) adding a mixture of an agent that induces nucleation in ethanol to the crystallization reactor of step (III-Axi′″);
(III-Axiii′″) removing the ethanol and dichloromethane of the crystallization reactor of Step (III-Axii″);
(III-Axiv′″) adding ethanol to the crystallization reactor of Step (III-Axiii″) then removing the ethanol;
(III-Axv′″) repeating Step (III-Axiv″) one or more times;
(III-Axvi′″) cooling the crystallization reactor of Step (III-Axv′″) to a temperature of about 18-25° C., agitating the crystallization reactor to provide a slurry;
(III-Axvii′″) filtering the slurry of Step (III-Axvi′″) and rinsing the resulting cake with ethanol; and
(III-Axviii “′) drying the cake of Step (III-Axvii′″) to provide crystalline Compound A.

In some embodiments, the agent that induces nucleation of Step (III-Ai”′) and/or Step (III-Axii′″) is a crystallization promoter. In some embodiments, the agent that induces nucleation of Step (III-Ai′″) and/or Step (III-Axii′″) is a seed crystal of Compound A.

In some embodiments, the purified crystalline Compound A is at least 95%, at least 96%, at least 97%, at least 98%, or at least 99% pure.

In some embodiments, the purified crystalline Compound A is at least 95%, at least 96%, at least 97%, at least 98%, or at least 99% pure as measured by HPLC.

In one aspect, the present disclosure is directed to a method comprising Step (III″ ″): (III″″) reacting Intermediate I-4:

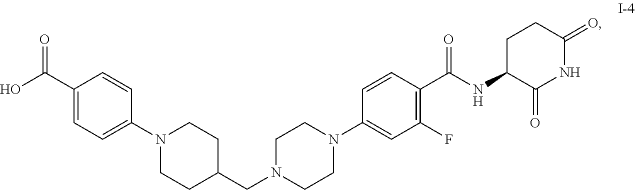 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

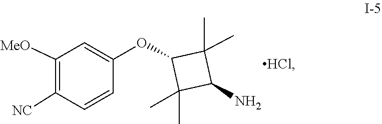 [SMILES: COc1cc(O[C@H]2C(C)(C)[C@H](N)C2(C)C)ccc1C#N]

in a reaction mixture, wherein the reaction mixture comprises a base, a coupling reagent, and an additive in a solvent to provide crystalline Compound A:

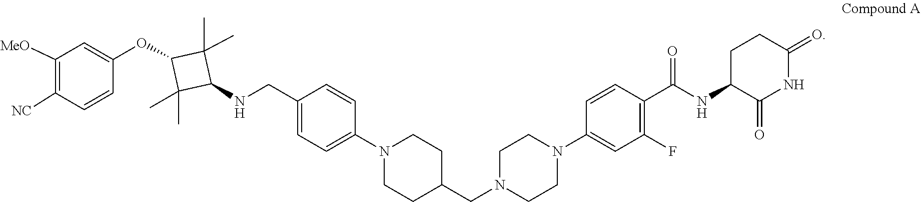 [SMILES: COc1cc(O[C@H]2C(C)(C)[C@H](NCc3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Step (III″ “′) provides Compound A in crystalline form.

In some embodiments, the salt of Intermediate I-4 is a bis-hydrochloride salt.

In some embodiments, the base of Step (III” “′) is an amine base.

In some embodiments, the base of Step (III″″) is N-methylmorpholine, trimethylamine, triethylamine, N,N-diisopropylethylamine, or N,N-dimethylaniline.

In some embodiments, the molar ratio of the base of Step (III″″) to Intermediate I-4 is about 1:1 to about 10:1.

In some embodiments, the molar ratio of the base of Step (III” “′) to Intermediate I-4 is about 3:1 to about 8:1, or preferably about 4.5:1 to about 6.5:1.

In some embodiments, the molar ratio of the base of Step (III″″) to Intermediate I-4 is about 1.0:1, about 1.5:1, about 2.0:1, about 2.5:1, about 3.0:1, about 3.5:1, about 4.0:1, about 4.5:1, about 5.0:1, about 5.5:1, about 6.0:1, about 6.5:1, about 7.0:1, about 7.5:1, about 8.0:1, about 8.5:1, about 9.0:1, about 9.5:1, about 10.0:1.

In some embodiments, the coupling reagent of Step (III″″) is a carbodiimide.

In some embodiments, the coupling reagent of Step (III″″) is 1-Ethyl-3-(3′-dimethylaminopropyl) carbodiimide or a salt thereof.

In some embodiments, the coupling reagent of Step (III″″) is 1-Ethyl-3-(3′-dimethylaminopropyl) carbodiimide hydrochloride.

In some embodiments, the molar ratio of the coupling reagent of Step (III″″) to Intermediate I-4 is about 1:1 to about 2:1.

In some embodiments, the molar ratio of the coupling reagent of Step (III″″) to Intermediate I-4 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

In some embodiments, the additive of Step (III″ “′) is ethyl 2-(hydroxyimino) cyanoacetate (OXYMA), 2-hydroxypyridine 1-oxide (HOPO), hexafluorophosphate azabenzotriazole tetramethyl uronium (HATU), or hydroxybenzotriazole (HOBt).

In some embodiments, the additive of Step (III″″) is ethyl 2-(hydroxyimino) cyanoacetate (OXYMA) or 2-hydroxypyridine 1-oxide (HOPO).

In some embodiments, the molar ratio of the additive of Step (III″″) to Intermediate I-4 is about 0.1:1 to about 0.5:1.

In some embodiments, the molar ratio of the additive of Step (III” “′) to Intermediate I-4 is about 0.10:1, about 0.15:1, about 0.2:1, about 0.25:1, about 0.30:1, about 0.35:1, about 0.40:1, about 0.45:1, about 0.50:1.

In some embodiments, the solvent of Step (III″″) is a polar solvent.

In some embodiments, the solvent of Step (III″″) is dimethylacetamide, dimethylsulfoxide, N-methyl-2-pyrrolidone, 2-methyltetrahydrofuran, dichloromethane, or any combination thereof.

In some embodiments, the solvent of Step (III″″) is dimethylacetamide.

In some embodiments, the solvent of Step (III″″) is dimethylsulfoxide.

In some embodiments, the solvent of Step (III″″) is N-methyl-2-pyrrolidone.

In some embodiments, the solvent of Step (III″″) is 2-methyltetrahydrofuran.

In some embodiments, the solvent of Step (III″″) is dichloromethane.

In some embodiments, Step (III″″) is conducted at a temperature of about −30° C. to about 30° C., or about −10° C. to about 10° C.

In some embodiments, Step (III″ “′) is conducted at a temperature of about −30° C., about-25° C., about −20° C., about −15° C., about −10° C., about −5° C., about 0° C., about 5° C., about 10° C., about 15° C., about 20° C., about 25° C., or about 30° C.

In some embodiments, Step (III″″) further comprises Step (III-ia″″), Step (III-iia″″), Step (III-iiia″″), Step (III-iva″ ″), Step (III-va″ ″), Step (III-via” “′), Step (III-viia″ ″), Step (III-viiia″″), Step (III-ixa” “′), and Step (III-xa″″):
(III-ia” “′) adding an organic solvent and water and to the reaction mixture comprising crude Compound A, stirring for several minutes, and allowing the reaction mixture to separate into organic and aqueous layers;
(III-iia″″) removing the organic layer from Step (III-ia″″) and washing the organic layer with a brine solution;
(III-iiia″″) removing and filtering the washed organic layer from Step (III-iia″″);
(III-iva″′) adding a second solvent and an agent that induces nucleation to the filtrate of Step (III-iiia″″);
(III-va″″) removing the second solvent of Step (III-iva″″) to provide a concentrated mixture;
(III-via″″) adding the second solvent of Step (III-iva″′) to the concentrated mixture of Step (III-va″″) and removing the second solvent to provide a concentrated mixture, optionally repeating the process one or more times;
(III-viia″″) cooling the concentrated mixture of Step (III-via″″) mixture to a temperature of about 18-25° C. over a period of about one hour;
(III-viiia″″) agitating the concentrated mixture of Step (III-viia″″) for about 8 hours or more at a temperature of about 18-25° C. to produce a slurry;
(III-ixa″″) filtering the slurry of Step (III-viiia″′) to provide a cake and rinsing the resulting cake with the second solvent of Step (III-iva″″); and
(III-xa″″) drying the resulting cake of Step (III-ixa″″) by removing the second solvent to provide crystalline Compound A.

In some embodiments, the organic solvent of Step (III-ia″″) is dichloromethane.

In some embodiments, the brine solution of Step (III-iia″′) is half saturated brine (i.e., about 13% NaCl (w/v) in water).

In some embodiments, the agent that induces nucleation of step (III-iva″″) is a crystallization promoter.

In some embodiments, the crystallization promoter of Step (III-iva″″) is a seed crystal of Compound A.

In some embodiments, the second solvent of Step (III-iva″″) is an alcohol.

In some embodiments, the second solvent of Step (III-iva″″) is ethanol or methanol.

In some embodiments, the second solvent of Step (III-va″″), Step (III-via″″), and Step (III-xa″″″) is removed by distillation.

In some embodiments, the second solvent of Step (III-va″ ″), Step (III-via″″), and Step (III-xa″″) is removed by vacuum distillation.

In some embodiments, the second solvent of Step (III-va″ ″), Step (III-via″″), and Step (III-viiia″″) is removed by atmospheric distillation.

In some embodiments, the purified crystalline Compound A is at least 95%, at least 96%, at least 97%, at least 98%, or at least 99% pure.

In some embodiments, the purified crystalline Compound A is at least 95%, at least 96%, at least 97%, at least 98%, or at least 99% pure as measured by HPLC.

In one aspect, the present disclosure is directed to a method wherein Intermediate I-4:

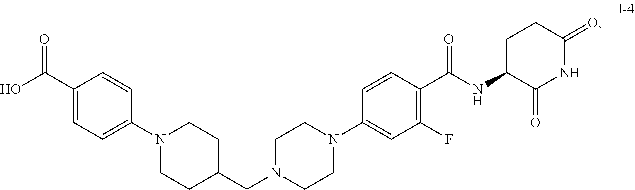 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, is prepared by a method comprising reacting Intermediate I-3:

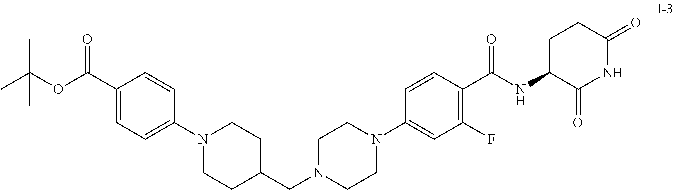 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid in a reaction mixture comprising a first solvent.

In one aspect, the present disclosure is directed to a method wherein Intermediate I-3 is prepared by a method comprising reductively aminating Intermediate I-1:

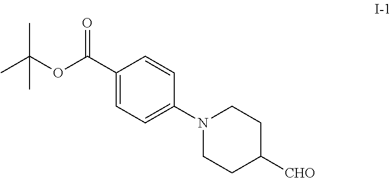 [SMILES: CC(C)(C)OC(=O)c1ccc(N2CCC(C=O)CC2)cc1]

with Intermediate I-2:

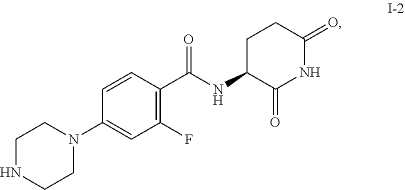 [SMILES: O=C1CC[C@H](NC(=O)c2ccc(N3CCNCC3)cc2F)C(=O)N1]

or a salt thereof, in a reaction mixture comprising a base, a reducing agent, and a solvent to provide wet Intermediate I-3:

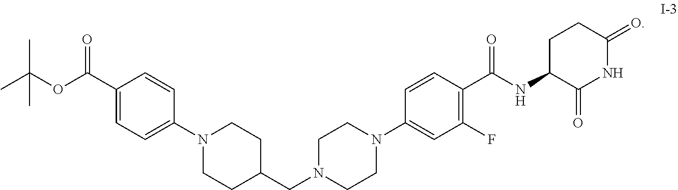 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In one aspect, the present disclosure is directed to a method of preparing Compound

A, comprising:
(a) reacting Intermediate I-1:

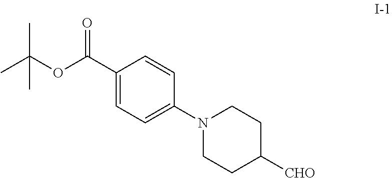 [SMILES: CC(C)(C)OC(=O)c1ccc(N2CCC(C=O)CC2)cc1]

with Intermediate I-2:

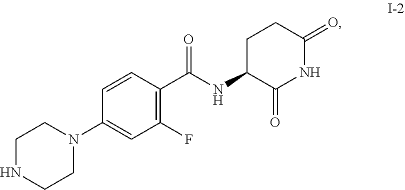 [SMILES: O=C1CC[C@H](NC(=O)c2ccc(N3CCNCC3)cc2F)C(=O)N1]

or a salt thereof,
in a first reaction mixture comprising a base, a reducing agent, and a solvent to provide wet Intermediate I-3:

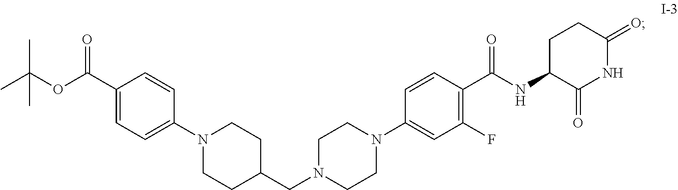 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

(b) drying wet Intermediate I-3;
(c) reacting dried Intermediate I-3:

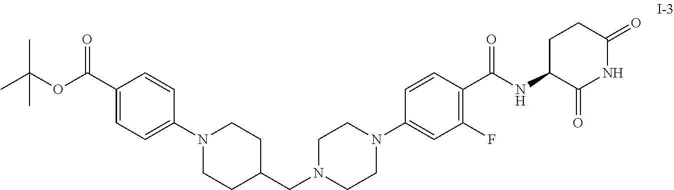 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof,
with an acid in a second reaction mixture to provide Intermediate I-4:

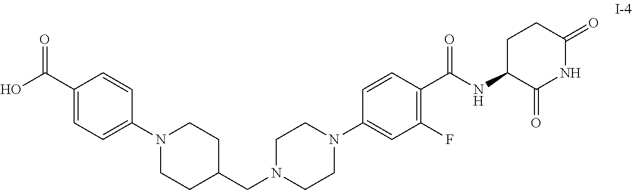 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
wherein the second reaction mixture comprises a second solvent;
(d) reacting Intermediate I-4

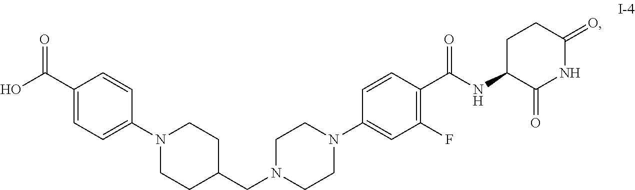 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

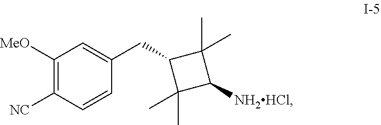

in a third reaction mixture, wherein the reaction mixture comprises a base, a coupling reagent, and an additive in a third solvent to provide crude Compound A:

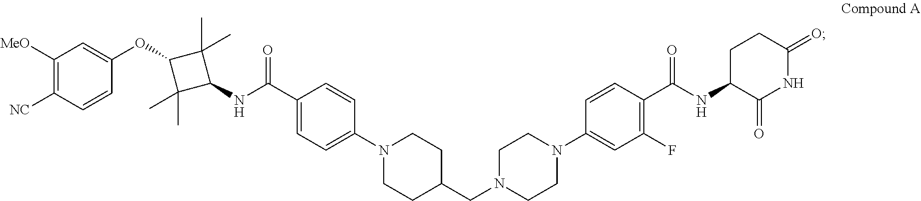 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and, optionally, wherein crude Compound A is purified by recrystallization.

In one aspect, the present disclosure is directed to a compound which is: tert-butyl(S)-4-(4-((4-(4-((2,6-dioxopiperidin-3-yl) carbamoyl)-3-fluorophenyl) piperazin-1-yl)methyl) piperidin-1-yl)benzoate (Intermediate I-3):

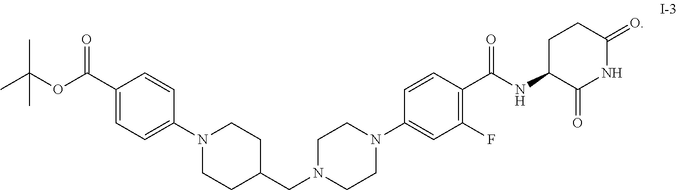 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In one aspect, the present disclosure is directed to a preparation of Intermediate I-3, wherein Intermediate I-3 in the preparation has a purity of greater than about 95%, greater than about 96%, or greater than about 97%.

In one aspect, the present disclosure is directed to a preparation of Intermediate I-3, wherein Intermediate I-3 in the preparation has a purity of greater than about 95%, greater than about 96%, or greater than about 97%, as measured by HPLC.

In one aspect, the present disclosure is directed to a compound which is: tert-butyl(S)-4-(4-((4-(4-((2,6-dioxopiperidin-3-yl) carbamoyl)-3-fluorophenyl) piperazin-1-yl)methyl) piperidin-1-yl)benzoate (Intermediate I-3):

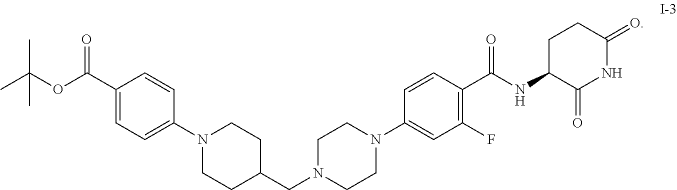 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In one aspect, the present disclosure is directed to a preparation of Intermediate I-3, wherein Intermediate I-3 in the preparation has a purity of greater than about 95%, greater than about 96%, or greater than about 97%.

In one aspect, the present disclosure is directed to a preparation of Intermediate I-3, wherein Intermediate I-3 in the preparation has a purity of greater than about 95%, greater than about 96%, or greater than about 97%, as measured by HPLC.

In one aspect, the present disclosure is directed to a compound which is: (S)-4-(4-((4-(4-((2,6-dioxopiperidin-3-yl) carbamoyl)-3-fluorophenyl) piperazin-1-yl)methyl) piperidin-1-yl)benzoic acid (Intermediate I-4),

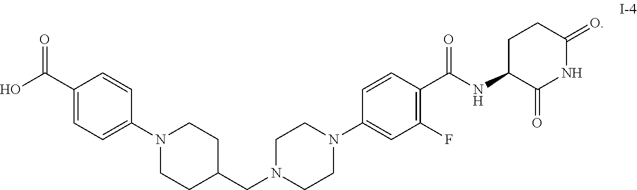 [SMILES: O=C1CC[C@H](NC(=O)c2ccc(N3CCN(CC4CCN(c5ccc(C(=O)O)cc5)CC4)CC3)cc2F)C(=O)N1]

In one aspect, the present disclosure is directed to a preparation of Intermediate I-4, wherein Intermediate I-4 in the preparation has a purity of greater than about 95%, greater than about 96%, greater than about 97%, or greater than about 98%.

In one aspect, the present disclosure is directed to a preparation of Intermediate I-4, wherein Intermediate I-4 in the preparation has a purity of greater than about 95%, greater than about 96%, greater than about 97%, or greater than about 98% as measured by HPLC.

In one aspect, the present disclosure is directed to a preparation of 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide (Compound A),

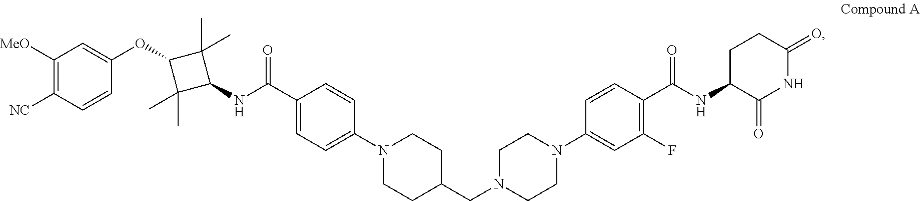 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein Compound A in the preparation has a purity of greater than about 95%, greater than about 96%, greater than about 97%, or greater than about 98%.

In one aspect, the present disclosure is directed to a preparation of 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide (Compound A),

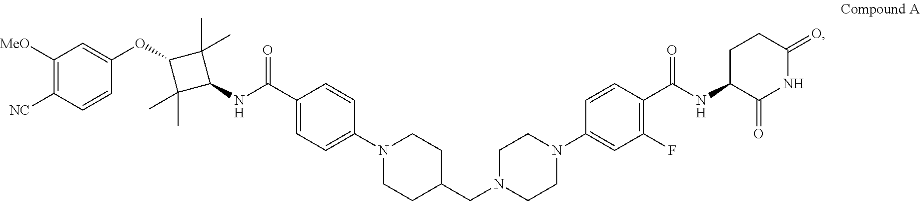 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein Compound A in the preparation has a purity of greater than about 95%, greater than about 96%, greater than about 97%, or greater than about 98%, as measured by HPLC.

In one aspect, the present disclosure is directed to a preparation of Compound A, wherein Compound A in the preparation has a purity of greater than about 98%, and comprises less than about 0.2%, less than about 0.3%, less than about 0.4%, or less than about 0.5% of impurity Intermediate I-3:

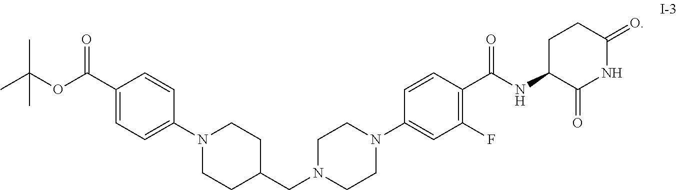 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

In one aspect, the present disclosure is directed to a preparation of Compound A, wherein Compound A in the preparation has a purity of greater than about 98%, and comprises less than about 0.2%, less than about 0.3%, less than about 0.4%, or less than about 0.5% of impurity Intermediate I-4:

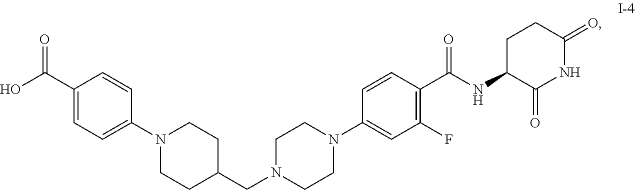 [SMILES: O=C1CC[C@H](NC(=O)c2ccc(N3CCN(CC4CCN(c5ccc(C(=O)O)cc5)CC4)CC3)cc2F)C(=O)N1]

as measured by HPLC.

In one aspect, the present disclosure is directed to a preparation of Compound A, wherein Compound A in the preparation has a purity of greater than about 98%, and comprises less than about 0.2%, less than about 0.3%, less than about 0.4%, or less than about 0.5% of impurity Intermediate I-3:

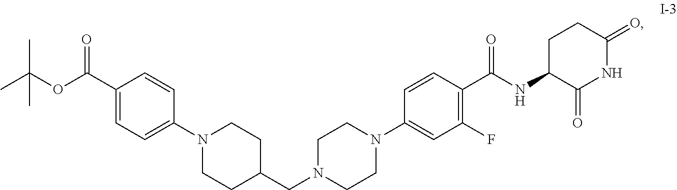 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

as measured by HPLC.

In one aspect, the present disclosure is directed to a preparation of Compound A, wherein Compound A in the preparation has a purity of greater than about 98%, and comprises less than about 0.2%, less than about 0.3%, less than about 0.4%, or less than about 0.5% of impurity Intermediate I-4:

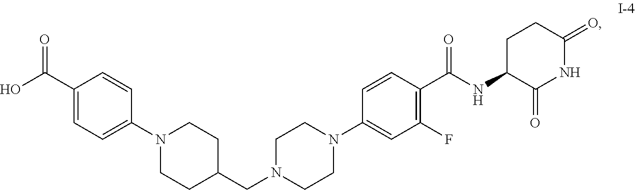 [SMILES: O=C1CC[C@H](NC(=O)c2ccc(N3CCN(CC4CCN(c5ccc(C(=O)O)cc5)CC4)CC3)cc2F)C(=O)N1]

as measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of Compound B:

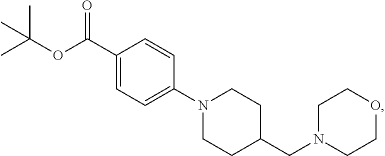 [SMILES: CC(C)(C)OC(=O)c1ccc(N2CCC(CN3CCOCC3)CC2)cc1]

as measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of Compound C:

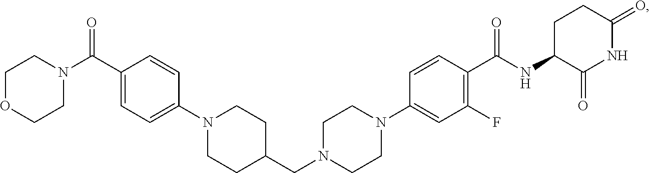 [SMILES: O=C1CC[C@H](NC(=O)c2ccc(N3CCN(CC4CCN(c5ccc(C(=O)N6CCOCC6)cc5)CC4)CC3)cc2F)C(=O)N1]

as measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of Compound D:

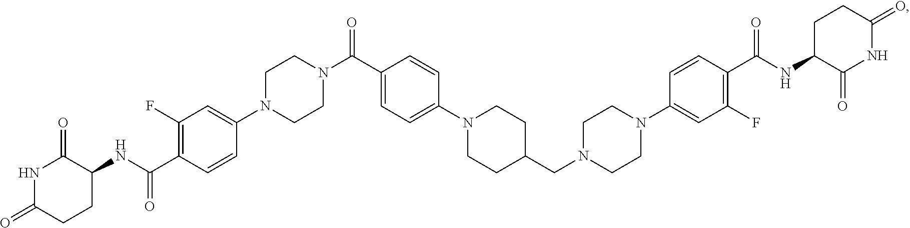 [SMILES: O=C1CC[C@H](NC(=O)c2ccc(N3CCN(CC4CCN(c5ccc(C(=O)N6CCN(c7ccc(C(=O)N[C@H]8CCC(=O)NC8=O)c(F)c7)CC6)cc5)CC4)CC3)cc2F)C(=O)N1]

as measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of Compound E:

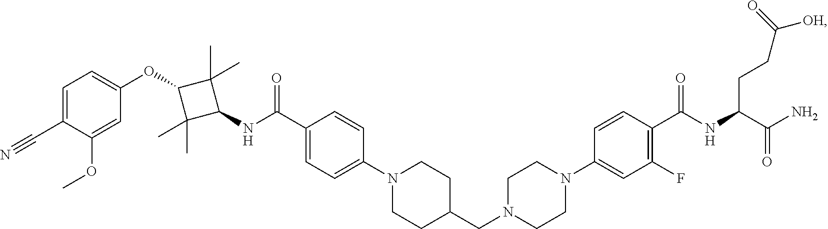 [SMILES: COc1cc(O[C@H]2C(C)(C)[C@H](NC(=O)c3ccc(N4CCC(CN5CCN(c6ccc(C(=O)N[C@@H](CCC(=O)O)C(N)=O)c(F)c6)CC5)CC4)cc3)C2(C)C)ccc1C#N]

measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of Compound F:

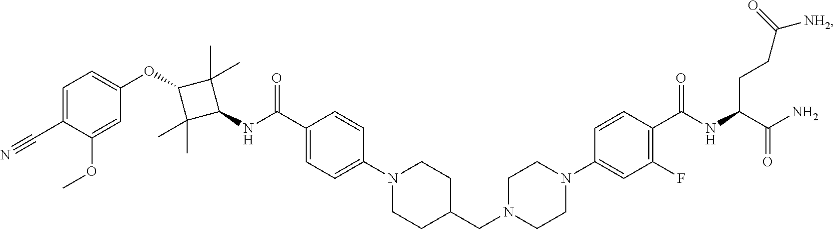 [SMILES: COc1cc(O[C@H]2C(C)(C)[C@H](NC(=O)c3ccc(N4CCC(CN5CCN(c6ccc(C(=O)N[C@@H](CCC(N)=O)C(N)=O)c(F)c6)CC5)CC4)cc3)C2(C)C)ccc1C#N]

as measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of impurity Compound G:

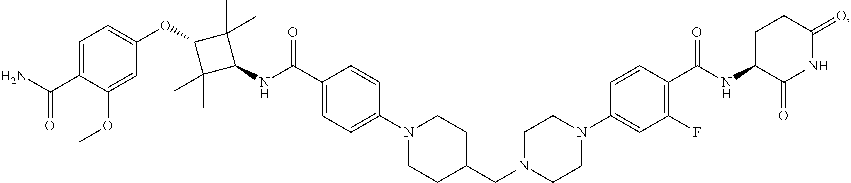 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C(N)=O]

as measured by HPLC.

In one aspect, the present disclosure is related to a preparation of Compound A, wherein Compound A in the preparation has a purity of greater than about 98%, and comprises less than about 0.1%, less than about 0.2%, less than about 0.3%, less than about 0.4%, or less than about 0.5% of:

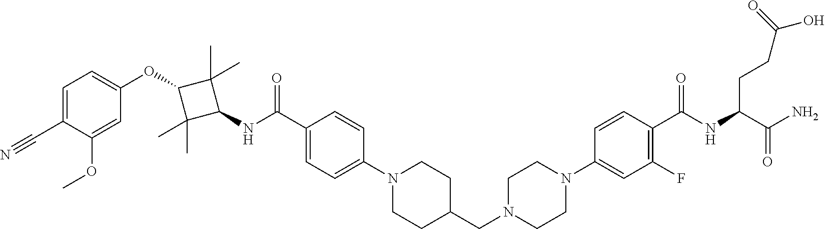 [SMILES: COc1cc(O[C@H]2C(C)(C)[C@H](NC(=O)c3ccc(N4CCC(CN5CCN(c6ccc(C(=O)N[C@@H](CCC(=O)O)C(N)=O)c(F)c6)CC5)CC4)cc3)C2(C)C)ccc1C#N]

Compound E,

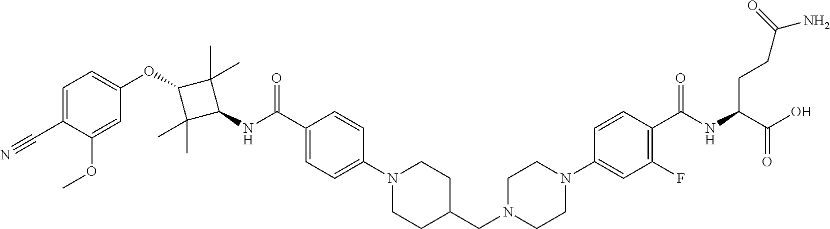 [SMILES: COc1cc(O[C@H]2C(C)(C)[C@H](NC(=O)c3ccc(N4CCC(CN5CCN(c6ccc(C(=O)N[C@@H](CCC(N)=O)C(=O)O)c(F)c6)CC5)CC4)cc3)C2(C)C)ccc1C#N]

Compound F, or a combination thereof, as measured by HPLC.

### Processes of Manufacturing Compound A

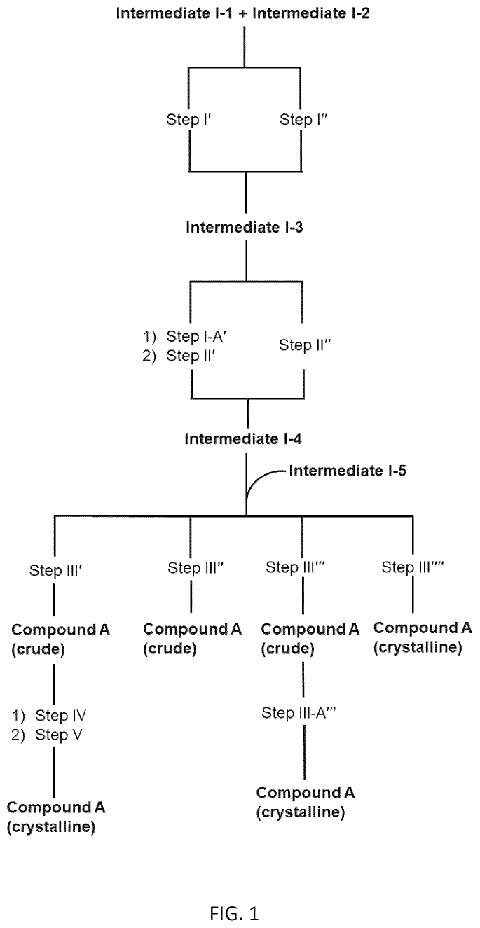

The processes for manufacturing Compound A in the disclosure are summarized in FIG. 1 and below in Schemes 1 and 2.

In some embodiments, Compound A is manufactured via Process 1, which is summarized below in Scheme 1 and described in the Examples section.

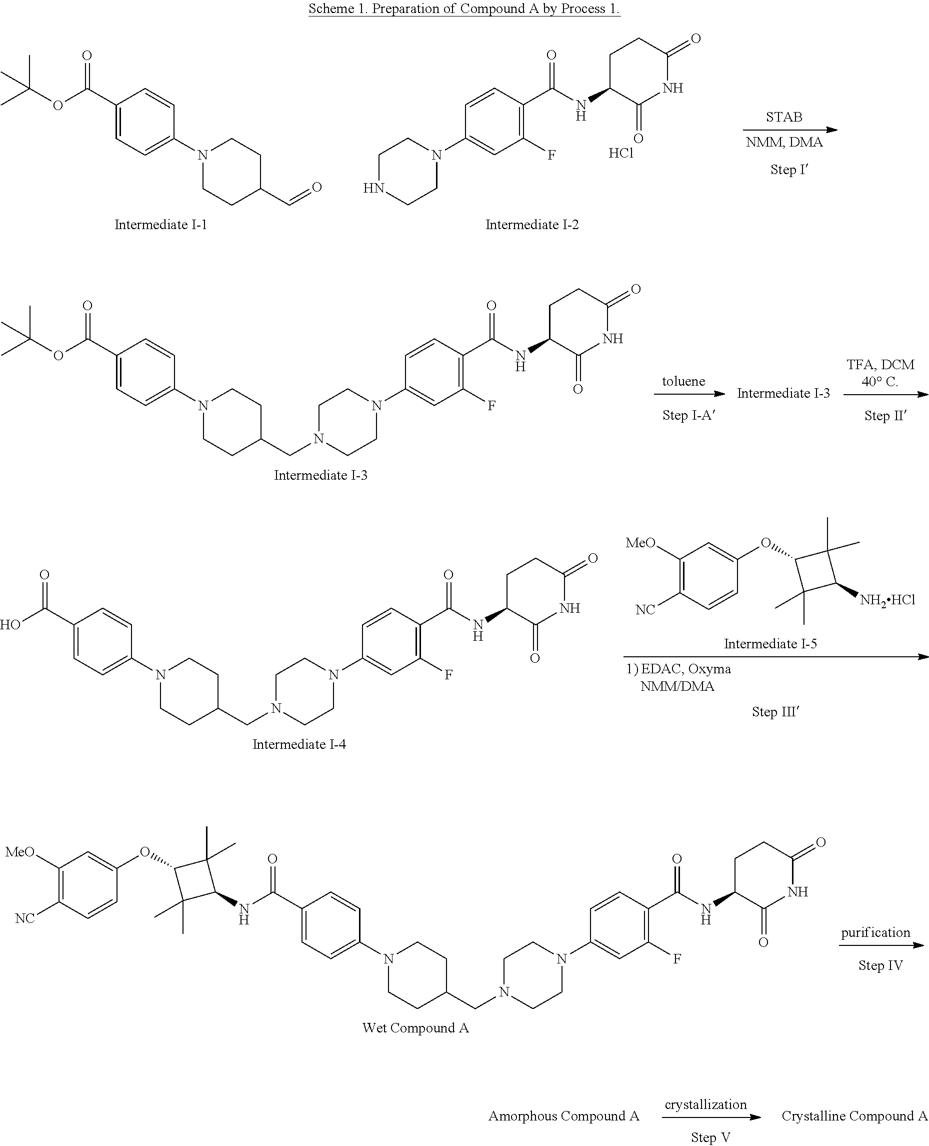

In some embodiments, Compound A is manufactured via Process 2, which is summarized below in Scheme 2 and described in the Examples section.

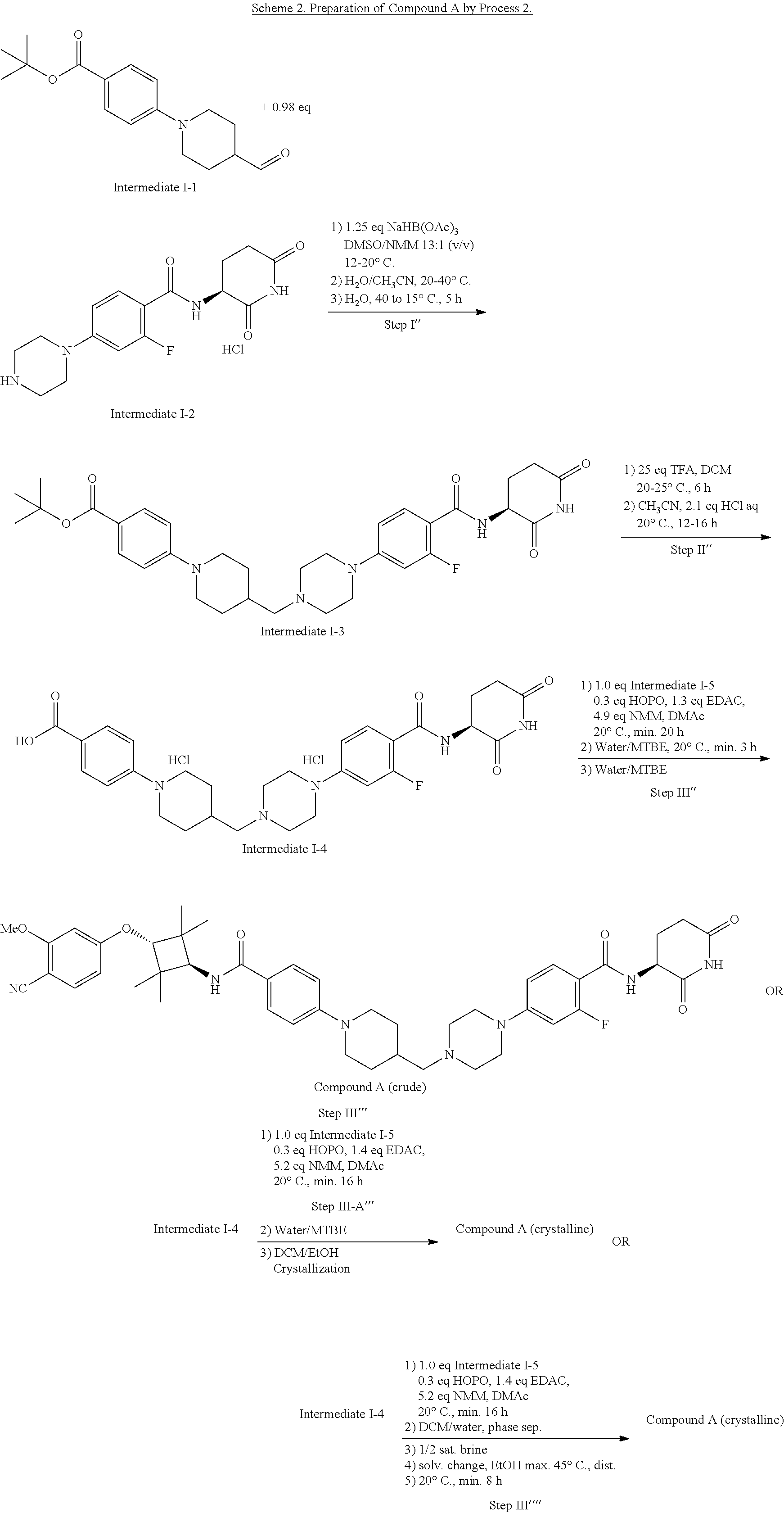

In some embodiments, Step I is Step I′ or Step I″.

In some embodiments, Step I is Step I′.

In some embodiments, Step I is Step I″.

In some embodiments, Step II is Step I-A′ and Step II′ or Step II″.

In some embodiments, Step II is Step I-A′ and Step II′.

In some embodiments, Step II is Step II″.

In some embodiments, Step III is Step III′, Step III″, Step III″, Step III″ and Step III-A′″, or Step III″ “.

In some embodiments, Step III is Step III′.

In some embodiments, Step III is Step III”.

In some embodiments, Step III is Step III″.

In some embodiments, Step III is Step III″ and Step III-A″.

In some embodiments, Step III is Step III″ ″.

### Definitions

Unless otherwise defined, all technical and scientific terms used herein have the same meaning as commonly understood by one of ordinary skill in the art to which this disclosure belongs. The terminology used in this description is intended to describe particular embodiments only and is not intended to limit the disclosure.

Where a range of values is disclosed herein, it is understood that the present disclosure encompasses each intervening value, to the tenth of the unit of the lower limit unless the context clearly dictates otherwise (such as in the case of a group containing a number of carbon atoms; in such cases, each integer falling within the range is provided), from the upper to the lower limit of that range, and any other stated or intervening value in that stated range. As a non-limiting example, the range of 1-10 encompasses each of 1.0, 1.1. 1.2, 1.3, etc. The upper and lower limits of these smaller ranges may independently be included in the smaller ranges, and these are also encompassed within the present disclosure, subject to any specifically excluded limit in the stated range. Where the stated range includes one or both limits, ranges excluding either or both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

As used herein, the phrase “and/or” should be understood to mean “both or either” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also optionally including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both″) when preceded by terms of exclusivity, such as “either,” “one of,” “only one of,” or “exactly one of.”

In the claims, as well as in the specification above, all transitional phrases such as “comprising,” “including,” “carrying.” “having,” “containing,” “involving,” “holding,” “composed of,” and the like are to be understood to be open-ended, i.e., to include but not be limited to. Only the transitional phrases “consisting of” and “consisting essentially of” shall be closed or semi-closed transitional phrases, respectively, as set forth in the United States Patent Office Manual of Patent Examining Procedures, Ninth Edition, Revision 10.2019, Section 2111.03.

As used herein in the specification and in the claims, the phrase “at least one,” in reference to a list of one or more elements, means at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a nonlimiting example, “at least one of A and B” (or, equivalently, “at least one of A or B,” or, equivalently “at least one of A and/or B″) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” and the like, as used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. Thus, the term “about” is normally used to encompass values within standard error. In one embodiment, the term “about” as part of a quantitative expression such as “about X”, includes any value that is up to 10% higher or lower than X, and also includes any numerical value that falls between X-10% and X+10% (e.g., X-5% and X+5%, or X-3% and X+3%). Thus, for example, a weight of about 40 g may include a weight of between 36 to 44 g, inclusive of the endpoints; a temperature of about 100° C. may include a temperature of 90° C. to 110° C., inclusive of endpoints; and a temperature range of about 90-100° C., may include a range of 81-110° C., inclusive of the endpoints. Thus, for example, a percent composition of about 50% may include a percent composition of between 45% to 55%, inclusive of the endpoints.

As used herein, the term “mixture” or “reaction mixture” means a combination of more than one compound, usually within a solvent, that is about to undergo a chemical reaction, is in the process of undergoing a chemical reaction, or has undergone a chemical reaction.

As used herein, the term “reaction” means a process that leads to the chemical transformation of one set of chemical substances to another. As used herein, the term “to react” means to introduce chemical substances together to result in a chemical reaction.

As used herein, the term “cool” or “cooled” or “to cool” means to either passively allow by means of heat dissipation or act by using water or a heat sink (ice, dry ice, etc.) to actively decrease the temperature of an object, mixture, reaction mixture, concentrate, etc.

As used herein, the term “coupling reagent” or “coupling agent” refers to a compound that aids in bringing about a reaction to couple one compound to another compound. Coupling reagents may include, but are not limited to, chlorinating agents (as defined herein), propanephosphonic acid anhydride, 1,1′-carbonyldiimidazole, EDC/HOBt or other amide or ester coupling reagents, isobutyl chloroformate, and pivaloyl chloride (to generate a pivalate ester).

As used herein, the term “chlorinating agent” refers to an agent, compound, or element that introduces chlorine atoms into another compound. Chlorinating agents may include, but are not limited to, chlorine gas (Cl₂), N-chlorosuccinimide (NCS), and iodobenzene dichloride (PhICl₂).

As used herein, the term “reducing agent” means a compound that loses (or “donates”) an electron to an electron recipient (oxidizing agent) in a redox chemical reaction. Reducing agents include those generally known in the art including, for example, sodium hypophosphite (NaH₂PO₂), formaldehyde (CH₂O) and other aldehydes, formic acid (HCOOH), salts of formic acid, salts of borohydride (e.g., sodium borohydride (NaBH₄)), salts of substituted borohydrides (e.g., sodium triacetoxyborohydride (Na(CH₃CO₂)₃BH) and sodium cyanoborohydride (Na[BH₃(CN)]), sodium alkoxides, lithium aluminum hydride (LiAlH₄), diisobutyl aluminum hydride (DIBAH), hydrazine (H₂NNH₂), and ammonia. Also used for reduction is catalytic hydrogenation.

As used herein, the term “reaction product” refers a composition which results after reacting the reagents of one step of a method of the current disclosure. The term “reaction product” can refer to a purified chemical compound (e.g., a substantially pure compound such as a recrystallized salt), or a composition comprising one or more impurities (i.e., a crude reaction product).

As used herein, the term “crude” in “crude reaction product” or “crude form” refers to the product of a reaction (i.e., a compound) that has not been subjected to a purification step after its initial preparation (e.g., recrystallization, sublimation, flash column chromatography, HPLC, etc.). The crude reaction product or crude form of a compound may contain one or more impurities, including, for example, one or more of the impurities described herein.

As used herein, the phrase “agent that induces nucleation” or “nucleation agent” refers to any object, material or action that results in primary or secondary nucleation. Primary nucleation is the initial formation of a crystal where there are no other crystals present or where, if there are crystals present in the system, they do not have any influence on the crystallization process. This can occur in two conditions. The first is homogeneous nucleation, which is nucleation that is not influenced in any way by solids. These solids include the walls of the crystallizer vessel and particles of any foreign substance. The second category is heterogeneous nucleation, which occurs when solid particles of foreign substances (e.g., any substance that is physically or chemically distinct from the crystals to be formed) cause an increase in the rate of nucleation that would otherwise not occur without the presence of these foreign substances. Homogeneous nucleation rarely occurs in practice due to the high energy necessary to begin nucleation without a solid surface on which to catalyze the nucleation. Secondary nucleation is when crystal growth is initiated with contact of other existing crystals or “seeds”. The first type of known secondary crystallization is attributable to fluid shear. The second type is due to collisions between already existing crystals with either a solid surface of the crystallizer or with other crystals themselves. Other agents that induce nucleation include devices, such as a DTB crystallizer, an evaporative crystallizer, or cooling crystallizers (e.g., a Swenson-Walker crystallizer). In some embodiments, the agent that induces nucleation is a crystallization promoter.

As used herein, the term “crystallization promoter” means an action or material that can promote the solidification of a compound from solution. In some embodiments, “crystallization promoter” is a seed crystal. In some embodiments, “crystallization promoter” is a seed crystal of

Compound A. In some embodiments, the crystallization promoter could be obtained by scratching against a glass surface to provide surface area for crystallization. In some embodiments, sonication can promote the crystallization of a compound in solution. In some embodiments, evaporative or solvent transfer crystallization can be used. Solvent layering can promote the crystallization at the interface which in turn promotes crystallization of a compound of interest. Vapor diffusion (such as, e.g., hanging drop and sitting drop methods), and batch methods of crystallization, for example, can be used. A seed crystal can be a small piece of single crystal or polycrystal material from which a large crystal of typically the same material is to be grown. Used to replicate material, the use of a seed crystal to promote crystal growth avoids the otherwise slow randomness of natural crystal growth and allows manufacture on a scale suitable for industry. Other agents that promote crystallization include devices, such as a DTB crystallizer, an evaporative crystallizer, or cooling crystallizers (e.g., a Swenson-Walker crystallizer),

As used herein, the term “azeotropic removal” or “azeotropic distillation” refers to techniques that harness interactions between the components of the solution to create properties unique to the solution, as most processes entail non-ideal mixtures, where Raoult's law does not hold. Such interactions can result in a constant-boiling azeotrope which behaves as if it were a pure compound (i.e., boils at a single temperature instead of a range). As an azeotrope, the solution contains the given component in the same proportion as the vapor, so that evaporation does not change the purity, and distillation does not affect separation. For example, ethyl alcohol and water form an azeotrope of 95.6% at 78.1° C. If the azeotrope is not considered sufficiently pure for use, there exist some techniques to break the azeotrope to give a pure distillate. This set of techniques is known as azeotropic distillation. Some techniques achieve this by “jumping” over the azeotropic composition (by adding another component to create a new azeotrope, or by varying the pressure). Others work by chemically or physically removing or sequestering the impurity. For example, to purify ethanol beyond 95%, a drying agent (or desiccant, such as potassium carbonate) can be added to convert the soluble water into insoluble water of crystallization. Molecular sieves are often used for this purpose. Immiscible liquids, such as water and toluene, easily form azeotropes. Commonly, these azeotropes are referred to as a low boiling azeotrope because the boiling point of the azeotrope is lower than the boiling point of either pure component. The temperature and composition of the azeotrope is easily predicted from the vapor pressure of the pure components, without use of Raoult's law. The azeotrope is easily broken in a distillation set-up by using a liquid-liquid separator (a decanter) to separate the two liquid layers that are condensed overhead. Only one of the two liquid layers is refluxed to the distillation set-up. High boiling azeotropes, such as a 20 percent by weight mixture of hydrochloric acid in water, also exist. As implied by the name, the boiling point of the azeotrope is greater than the boiling point of either pure component.

As used herein, the term “supercritical fluid chromatography” or “SFC” means a form of normal phase chromatography that uses a supercritical fluid such as carbon dioxide as the mobile phase. It is used for the analysis and purification of low to moderate molecular weight, thermally labile molecules and can also be used for the separation of chiral compounds. Principles are similar to those of high-performance liquid chromatography (HPLC); however, SFC typically utilizes carbon dioxide as the mobile phase; therefore, the entire chromatographic flow path must be pressurized. Since the supercritical phase represents a state in which liquid and gas properties converge, supercritical fluid chromatography is sometimes called convergence chromatography. SFC with CO₂ utilizes carbon dioxide pumps that require that the incoming CO₂ and pump heads be kept cold to maintain the carbon dioxide at a temperature and pressure that keeps it in a liquid state where it can be effectively metered at some specified flow rate. The CO₂ subsequently becomes supercritical post the injector and in the column oven when the temperature and pressure it is subjected to are raised above the critical point of the liquid and the supercritical state is achieved. SFC as a chromatographic process has been compared to processes having the combined properties of the power of a liquid to dissolve a matrix, with the chromatographic interactions and kinetics of a gas. The result is a large mass per injection while maintaining high chromatographic efficiency. Typically, gradient elution is employed in analytical SFC using a polar co-solvent such as methanol, possibly with a weak acid or base at low concentrations ˜1%. The effective plate counts per analysis can be observed to exceed 500K plates per meter routinely with 5 μm material. The operator uses software to set mobile phase flow rate, co-solvent composition, system back pressure and column oven temperature which must exceed 40° C. for supercritical conditions to be achieved with CO₂. In addition, SFC provides an additional control parameter-pressure-by using an automated back pressure regulator. From an operational standpoint, SFC is as simple and robust as HPLC, but fraction collection is more convenient because the primary mobile phase evaporates leaving only the analyte and a small volume of polar co-solvent. If the outlet CO₂ is captured, it can be recompressed and recycled, allowing for >90% reuse of CO₂. Similar to HPLC, SFC uses a variety of detection methods including UV/VIS, mass spectrometry, FID (unlike HPLC) and evaporative light scattering.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

As used herein, the term “impurity” refers to an unwanted compound, trace metal, or solvent that contaminates Compound A. In one embodiment, the impurity is a compound selected from the group consisting of Intermediate 2, Intermediate 3, Intermediate 5, and Impurity 1. In one embodiment, the impurity is a solvent that is selected from the group consisting of dichloromethane, methanol, and acetonitrile.

“Stable”, as used herein with reference to Compound A, refers to forms of Compound A, including, crystalline forms and amorphous forms, that stably retain purity equal to, or greater than, 95%, 96%, 97%, 98%, 99%, or 99.5% over a period of time (such as 6 months, 12 months, or 24 months) and under specified conditions (e.g., temperature and humidity) (such as 4° C., 25° C., or 40° C.).

As used herein, “Compound A” refers to 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide, which has the following structure:

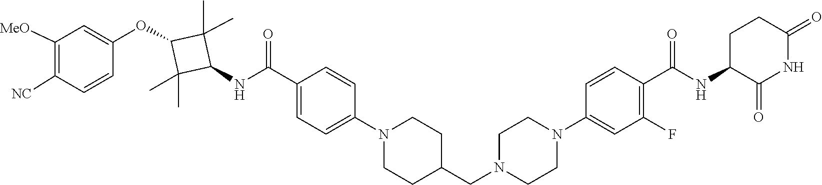 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and is described in U.S. Pat. No. 11,883,393, which is incorporated by reference herein in its entirety for all purposes.

As used herein, “epi-Compound A” refers to 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-vl)_N_ ((R)_? 6-dinvonineridin_3_v1)_?-fluorohenzamide which has the following structure:

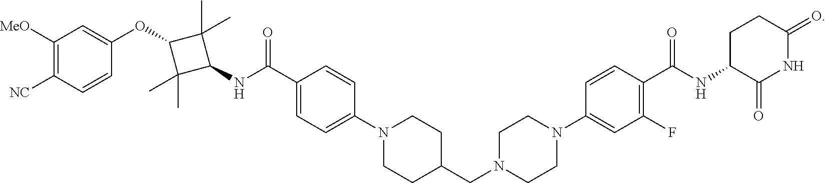 [SMILES: COc1cc(O[C@H]2C(C)(C)[C@H](NC(=O)c3ccc(N4CCC(CN5CCN(c6ccc(C(=O)N[C@@H]7CCC(=O)NC7=O)c(F)c6)CC5)CC4)cc3)C2(C)C)ccc1C#N]

As used herein, “Compound B” refers to tert-butyl 4-(4-(morpholinomethyl) piperidin-1-yl)benzoate, which has the following structure:

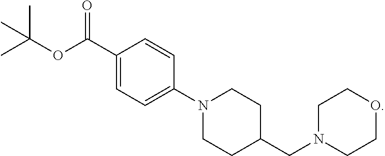 [SMILES: CC(C)(C)OC(=O)c1ccc(N2CCC(CN3CCOCC3)CC2)cc1]

As used herein, “Compound C” refers to(S)—N-(2,6-dioxopiperidin-3-yl)-2-fluoro-4-(4-((1-(4-(morpholine-4-carbonyl)phenyl) piperidin-4-yl)methyl) piperazin-1-yl)benzamide, which has the following structure:

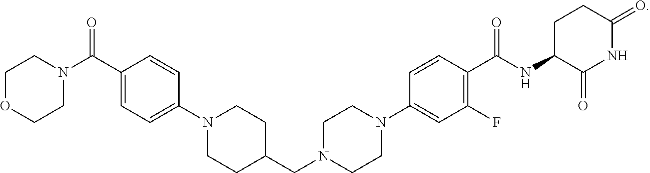 [SMILES: O=C1CC[C@H](NC(=O)c2ccc(N3CCN(CC4CCN(c5ccc(C(=O)N6CCOCC6)cc5)CC4)CC3)cc2F)C(=O)N1]

As used herein, “Compound D” refers to N-((S)-2,6-dioxopiperidin-3-yl)-4-(4-(4-(4-((4-(4-(((S)-2,6-dioxopiperidin-3-yl) carbamoyl)-3-fluorophenyl) piperazin-1-yl)methyl) piperidin-1-yl)benzoyl) piperazin-1-yl)-2-fluorobenzamide, which has the following structure:

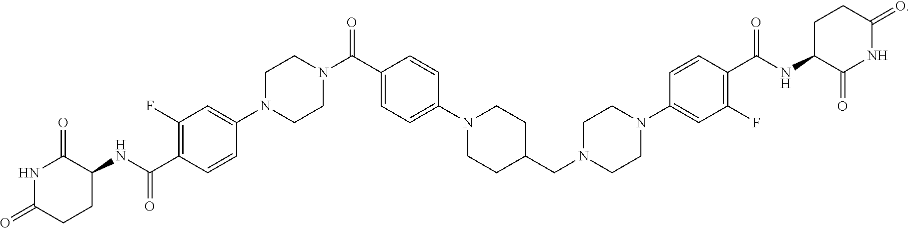 [SMILES: O=C1CC[C@H](NC(=O)c2ccc(N3CCN(CC4CCN(c5ccc(C(=O)N6CCN(c7ccc(C(=O)N[C@H]8CCC(=O)NC8=O)c(F)c7)CC6)cc5)CC4)CC3)cc2F)C(=O)N1]

As used herein, “Compound E” refers to(S)-5-amino-4-(4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-2-fluorobenzamido)-5-oxopentanoic acid, which has the following structure:

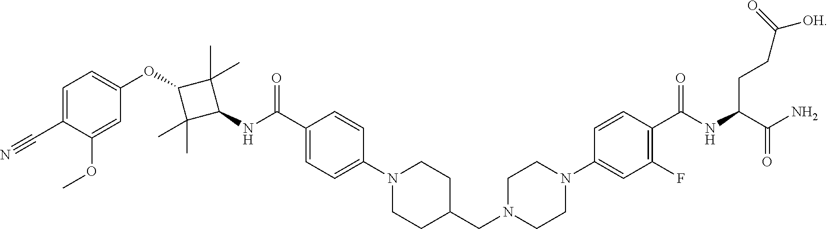 [SMILES: COc1cc(O[C@H]2C(C)(C)[C@H](NC(=O)c3ccc(N4CCC(CN5CCN(c6ccc(C(=O)N[C@@H](CCC(=O)O)C(N)=O)c(F)c6)CC5)CC4)cc3)C2(C)C)ccc1C#N]

As used herein, “Compound F” refers to (4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-2-fluorobenzoyl)-L-glutamine, which has the following structure:

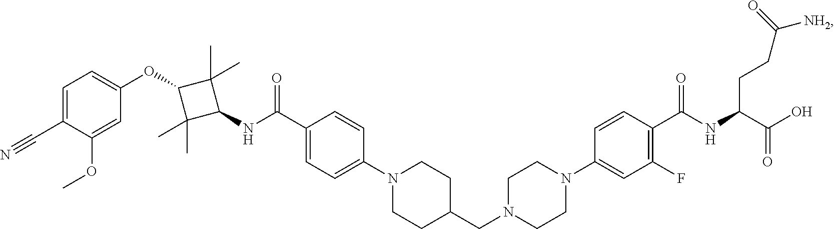 [SMILES: COc1cc(O[C@H]2C(C)(C)[C@H](NC(=O)c3ccc(N4CCC(CN5CCN(c6ccc(C(=O)N[C@@H](CCC(N)=O)C(=O)O)c(F)c6)CC5)CC4)cc3)C2(C)C)ccc1C#N]

As used herein, “Compound G” refers to 4-(4-((1-(4-(((1r,3r)-3-(4-carbamoyl-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide, which has the following structure:

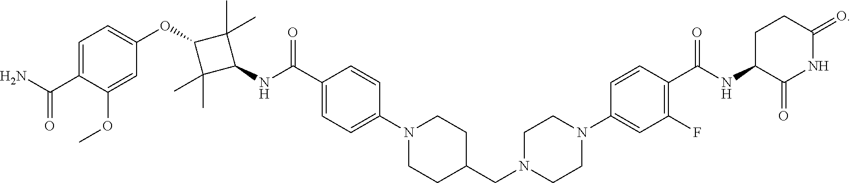 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C(N)=O]

As used herein, the term “preparation”, i.e., “a preparation of Compound A”, refers to a composition, e.g., a pharmaceutical composition or formulation such as a tablet or capsule, that includes a compound (e.g., Compound A) with a high level of purity, i.e., greater than about 95%, greater than about 96%, greater than about 97%, or greater than about 98%. In some embodiments, the preparation does not include or contain any detectable amounts of other compound(s), e.g., intermediates and/or impurities in the process for manufacturing Compound A. In some embodiments, the preparation includes less than specified amounts of other compound(s), e.g., intermediates and/or impurities in the process for manufacturing Compound A, i.e., less than about 0.2%, less than about 0.3%, less than about 0.4%, or less than about 0.5% of one or more intermediates or impurities.

All publications and patent documents cited herein are incorporated herein by reference as if each such publication or document were specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

| Abbreviation | Definition |
|---|---|
| Corr. | Corrected |
| DCM | Dichloromethane |
| DMAc | Dimethylacetamide |
| DMSO | Dimethyl sulfoxide |
| DSC | Differential scanning calorimetry |
| EDAC | 1-Ethyl-3-(3′-dimethylaminopropyl)carbodiimide, HCl |
| Eq | Equivalent |
| FT-IR | Fourier-transform infrared spectroscopy |
| GC-HS | Gas Chromatograph - Headspace Sampling |
| HDPE | High-density polyethylene |
| HOPO | 2-hydroxypyridine 1-oxide |
| HPLC | High-performance liquid chromatography |
| IPC | In-Process Control |
| ISO | International Standards Organization |
| KF | Karl Fischer titration |
| LOQ | Limit of Quantification |
| MTBE | Methyl tert-butyl ether |
| ML | Mother liquor |
| MW | Molecular Weight |
| N.D. | Not detected |
| NLT | Not less than |
| NMM | N-Methylmorpholine |
| QNMR | Quantitative NMR |
| RRT | Relative retention time |
| STAB | Sodium triacetoxyborohydride |
| TFA | Trifluoroacetic acid |
| USP | US Pharmacopeia |
| XRPD | X-ray Powder Diffraction |

The processes of the present invention will be better understood by reference to the following Examples, which are intended as an illustration of and not a limitation upon the scope of the application.

### Example 1. Synthesis of Intermediate I-3 (Step (I′))

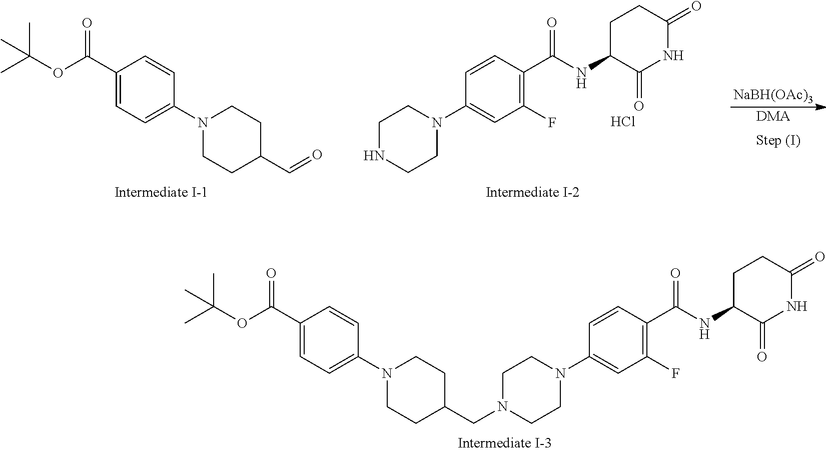

TABLE 1
List of Materials for Example 1

| | | | | | Actual | |
|---|---|---|---|---|---|---|
| Reagents/Materials | MW | Eqs. | Moles | Density | Amount | |
| Intermediate I-1 | 289.38 | 1.0 | 16.2 | — | 4.7 | kg |
| Intermediate I-2 | 370.1 | 1.0 | 16.2 | — | 6.0 | kg |
| Sodium | 211.94 | 1.5 | 24.4 | — | 5.2 | kg |
| Triacetoxyborohydride | | | | | | |
| (STAB) | | | | | | |
| Dimethylacetamide | — | 12v | — | 0.94 | 53 | kg |
| (DMAc), anhydrous | | | | | | |
| N-Methylmorpholine | 101.15 | 3.5 | 56.8 | — | 5.7 | kg |
| Process Water, filtered | 18.02 | 45v | — | 1.0 | 212 | kg |

Procedure: To a 400 L reactor charged,
53 kg Dimethylacetamide, anhydrous and started the agitator. To the reactor charged
4.7 kg Intermediate I-1,
6.0 kg Intermediate I-2,
5.7 kg N-Methylmorpholine (NMM) and agitated the contents in the reactor at 20±5° C. for at least 30 minutes. Contents of the reactor were then cooled≤5° C. and charged
5.2 kg Sodium Triacetoxyborohydride (STAB) and temperature was adjusted to 0±5° C. and contents were agitated for at least 16 hours.
   Withdrew a representative sample from the reactor.
   Analysis: Intermediate I-1:0.221%
      Intermediate I-2:0.58%
   The contents of the reactor were drained into clean HDPE drum(s) and the valve on the reactor was rinsed with at least
1 kg Dimethylacetamide, anhydrous. The reactor contents were drained into the same HDPE drum(s).
188 kg Process Water, filtered was charged to the reactor and was cooled to 5±5° C. Slowly charged Intermediate I-3 in DMAc/NMM to the reactor and rinsed the drum(s) with
2 kg Dimethylacetamide, anhydrous. The reactor was warmed to 20±5° C. and agitated for at least 16 hours.
   The reactor contents were filtered (˜26 hours) through the centrifuge and the cake was washed with
24 kg Process Water, filtered.
   Withdrew a representative sample from the reactor.
   Analysis: HPLC: 93.3%
      Chiral HPLC: 99.3%
   Intermediate I-3 was ready for the next step.

### Example 2. Isolation of Intermediate I-3 (Step (I-A′))

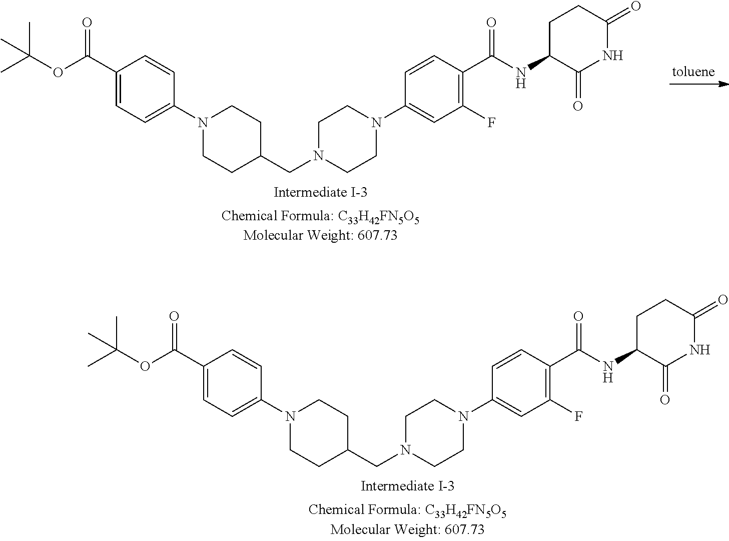 [SMILES: CC(C)(C)OC(=O)c1ccc(N2CCC(CN3CCN(c4ccc(C(=O)N[C@H]5CCC(=O)NC5=O)c(F)c4)CC3)CC2)cc1 ; CC(C)(C)OC(=O)c1ccc(N2CCC(CN3CCN(c4ccc(C(=O)N[C@H]5CCC(=O)NC5=O)c(F)c4)CC3)CC2)cc1]

TABLE 2
List of Materials for Example 2

| | | | | | Actual |
|---|---|---|---|---|---|
| Reagents/Materials | MW | Eqs. | Moles | Density | Amount |
| Intermediate I-3 | 607.73 | 1.0 | 46.1 | — | 28 kg |
| Toluene | 92.14 | 23v | — | 0.865 | 557 kg |

Procedure: To a 400 L reactor charged,
242 kg Toluene, started the agitator and charged wet Intermediate I-3. The contents agitated for at least 15 minutes at 20±5° C. Contents were then distilled until 43 L remained in the reactor. Jacket temperature on the reactor was cooled to 15° C. and through a polish filter charged
242 kg Toluene.
   Withdrew a 10 mL representative sample from the reactor.
   Filter the slurry (under nitrogen) to remove the toluene. Purge the sample with nitrogen. Place the sample in the container, purge the nitrogen and seal. Submit the wet cake (Intermediate I-3) for KF analysis.
   Analysis: KF: 1.6%
The contents of the reactor were then filtered and rinsed with polish filtered
73 kg Toluene.
   Intermediate I-3 was ready for the next step.

TABLE 3

| | | | KF | HPLC Purity | Chiral Purity |
|---|---|---|---|---|---|
| Batch | Yield | Yield | Expected | Expected | Expected |
| Number | (kg) | (%) | range: ≤5% | range: ≥95.0% | range: ≥98.5% |
| 2.1 | 6.15 kg | 62% | 2% | 97.4% | 99.7% desired |
| | | | | | 0.3% undesired |
| 2.2 | 6.50 kg | 65.7% | 1% | 97.2% | 99.6% desired |
| | | | | | 0.4% undesired |

### Example 3. Preparation of Intermediate I-4 (Step (II′))

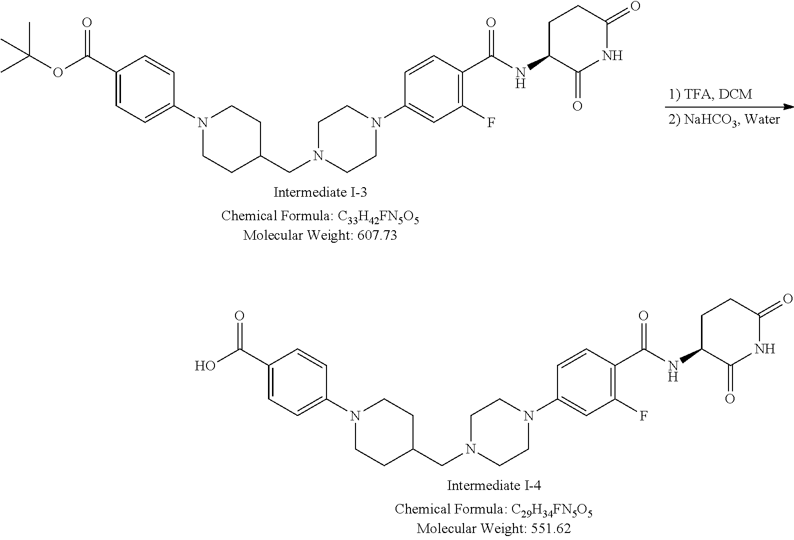 [SMILES: CC(C)(C)OC(=O)c1ccc(N2CCC(CN3CCN(c4ccc(C(=O)N[C@H]5CCC(=O)NC5=O)c(F)c4)CC3)CC2)cc1 ; O=C1CC[C@H](NC(=O)c2ccc(N3CCN(CC4CCN(c5ccc(C(=O)O)cc5)CC4)CC3)cc2F)C(=O)N1]

TABLE 4
List of Materials for Example 3

| | | | | | Actual | |
|---|---|---|---|---|---|---|
| Reagents/Materials | MW | Eqs. | Moles | Density | Amount | |
| Intermediate I-3 | 607.73 | 1.0 | 20.8 | — | 12.7 | kg |
| Dichloromethane | 84.93 | 2v | — | 1.33 | 34 | kg |
| (DCM) | | | | | | |
| Trifluoroacetic Acid | | 2v | — | — | 38 | kg |
| (TFA) | | | | | | |
| Acetonitrile (ACN) | 41.05 | 8v | — | 0.79 | 130 | kg |
| 7%(w/w) Sodium | — | 8v | — | — | 112 | kg |
| Bicarbonate, aqueous | | | | | | |
| solution | | | | | | |
| Process Water, filtered | 18.02 | 20v | — | — | 508 | kg |
| (cake wash-mid-process | | | | | | |
| filtration) | | | | | | |

Procedure: To a 400 L reactor charged,
34 kg DCM, started the agitator, charged
   Intermediate I-3 and heated the contents to 20±5° C. for at least 15 minutes.
   38 kg TFA was charged to the reactor and agitated for at least 15 minutes. The contents were then warmed to 40±5° C. and agitated for at least 10 hours.
   Withdrew a representative sample from the reactor.
   Analysis: Intermediate I-4:98.4%
      Intermediate I-3:1.6%
   Contents of the reactor were then cooled to 20±5° C. and the contents were distilled until 38 L remained in the reactor. Through a polish filter charged
2×50 kg Acetonitrile and distilled until 38 L remained in the reactor.
30 kg Acetonitrile was charged to the reactor and the temperature was adjusted to 20±5° C. Slowly charged to the reactor
12 kg 7% (w/w) Sodium Bicarbonate, aqueous solution. pH was <5.5.
7.3 kg Sodium Bicarbonate was charged to the reactor until the pH was between 5.5-6.5. Final pH was 5.8.
   Diaphragm pump was rinsed with
2 kg Process water, filtered and the contents of the reactor agitated for at least 15 hours. Contents of the reactor were then filtered via Centrifuge and rinsed with
254 kg Process Water, filtered. To the reactor charged
212 kg Toluene and started agitation. Charged wet Intermediate I-4 and agitated the contents for at least 15 minutes. The contents were the distilled until no water was visible in the dean stark. Reactor was cooled to 20±5° C. and sample was pulled for KF analysis (0.05%). Reactor contents were filtered and rinsed with polish filtered
64 kg Toluene. Intermediate I-4 was then dried at ≤45° C.
   Intermediate I-4 was then ready for the next step.

TABLE 5
Production Results for Example 3

| Batch | | | | | |
|---|---|---|---|---|---|
| Number | Yield (kg) | Yield (%) | HPLC Purity | Chiral Purity | KF |
| 3.1 | 12.2 | 106* | 99% | 100% | 3% |

*Not corrected for TFA Salts

### Example 4. Preparation of Compound A (Step (III′))

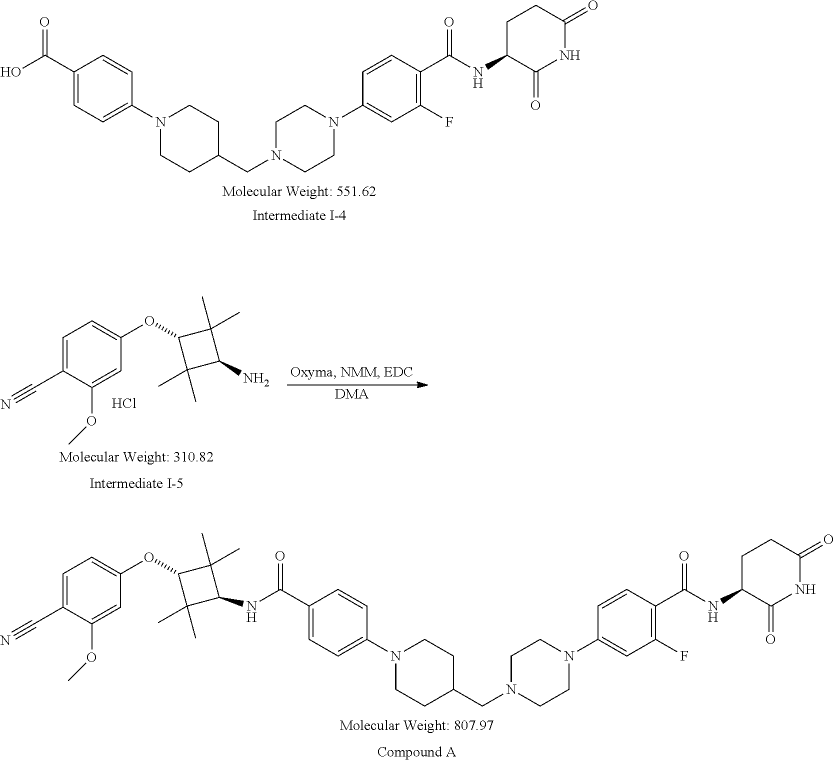

TABLE 6
List of Materials for Example 4

| | | | | | Actual | |
|---|---|---|---|---|---|---|
| Reagents/Materials | MW | Eqs. | Moles | Density | Amount | |
| Intermediate I-4 | 551.62 | 1.0 | 22.1 | — | 12.2 | kg |
| Intermediate I-5 | 310.82 | 1.05 | 23.2 | — | 7.2 | kg |
| Oxyma | 142.11 | 1.05 | 23.2 | — | 3.3 | kg |
| EDCI HCl | 191.70 | 1.55 | 34.2 | — | 6.6 | kg |
| Dimethylacetamide | — | 4v | — | — | 46 | kg |
| (DMAc) | | | | | | |
| N-Methylmorpholine | 101.15 | 5.5 | 121.6 | — | 12.3 | kg |
| Process Water, filtered | 18.02 | 47v | — | 1.00 | 573 | kg |
| Methyl tert- Butyl | 88.15 | 10v | — | 0.74 | 324 | kg |
| Ether (MTBE) | | | | | | |

Procedure: To a 400 L reactor charged,
46 kg Dimethylacetamide (DMAc) through a polish filter, started the agitator and cooled the contents to 0+5° C. Charged
12.2 kg Intermediate I-4,
3.3 kg Oxyma,
6.6 kg EDCI,
12.3 kg NMM through a polish filter and the contents agitated for at least 2.5-3.5 hours at 0±5° C. To the reactor charged
7.2 kg Intermediate I-5 and agitated the contents for at least 18 hours at 0±5° C.
   Withdrew a representative sample from the reactor.
   IPC Analysis: Intermediate I-4:0.12%
      Intermediate I-5:3.3%
      Compound A: 96.6%
   To the reactor, slowly charged through a polish filter, maintaining ≤5° C.
329 kg Process Water, filtered and then warmed the contents to 20±5° C. The reactor contents agitated for at least 15 hours and then filtered (˜70 hours) via centrifuge rinsing with
122 kg Process water, filtered and
162 kg MTBE.
   Wet Compound A was ready for the next step.

### Example 5. Purification of Compound A (Step (IV))

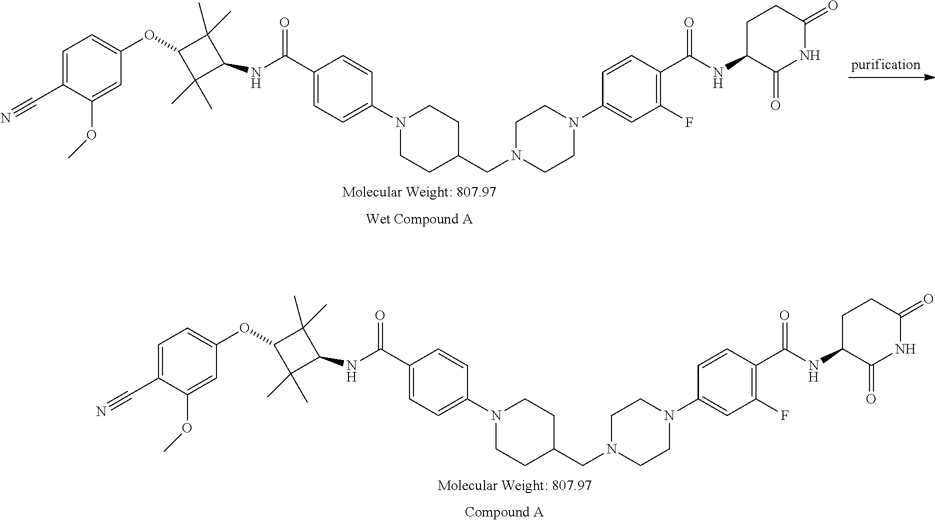 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N ; COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

TABLE 7
List of Materials for Example 5

| | | | | | Actual | |
|---|---|---|---|---|---|---|
| Reagents/Materials | MW | Eqs. | Moles | Density | Amount | |
| Compound A | 807.97 | 1.0 | 17.7 | — | 14.3 | kg |
| Acetonitrile | 41.05 | 20v | — | 0.79 | 224 | kg |
| 1:1 (v/v) | — | 10v | — | — | 134 | kg |
| Acetonitrile/Water | | | | | | |
| solution | | | | | | |

Procedure: To a 400 L reactor charged through a polish filter
154 kg Process Water, filtered and
135 kg Acetonitrile. The agitation was started and charged
   Wet Compound A and heated the contents to 31-36° C. and held the contents for at least 16 hours. Contents were the filtered through the centrifuge and rinsed with polish filtered
118 kg 1:1 (v/v) Acetonitrile/Water solution and
112 kg Acetonitrile.
   Withdrew a representative sample from the reactor.
   Analysis: HPLC: 98.1%
      Chiral: 97.4%
   Wet Compound A was ready for the next step.

### Example 6. Alternative Purification of Compound A (Step (IV))

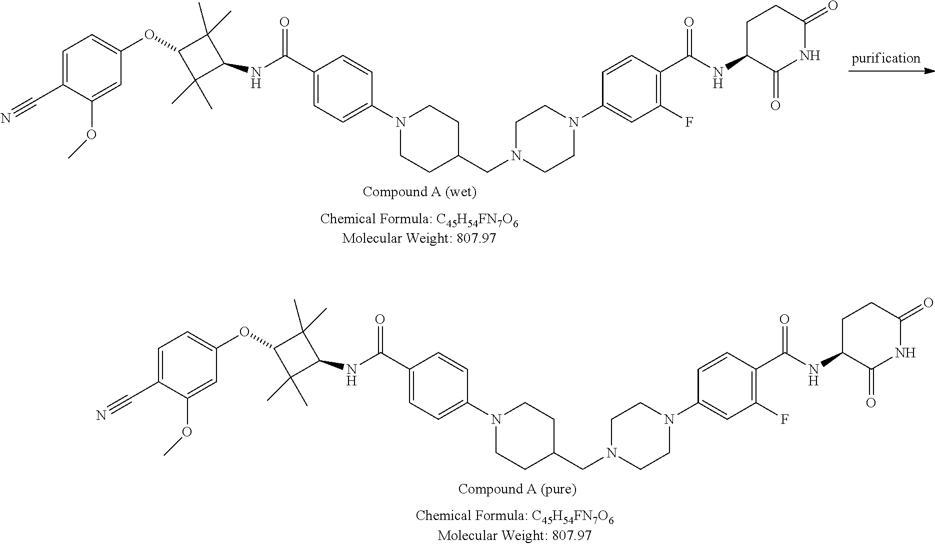 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N ; COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

TABLE 8
List of Materials for Example 6

| | | | | | Actual | |
|---|---|---|---|---|---|---|
| Reagents/Materials | MW | Eqs. | Moles | Density | Amount | |
| Compound A | 807.97 | 1.0 | 17.7 | — | 12.25 | kg |
| (theoretical amount) | | | | | | |
| Acetonitrile | 41.05 | 20v | — | 0.79 | 96 | kg |
| 1:1 (v/v) | — | 10v | — | — | 115 | kg |
| Acetonitrile/Water | | | | | | |
| solution | | | | | | |

Procedure: To a 400 L reactor charged through a polish filter
117 kg Process Water, filtered and
96 kg Acetonitrile. The agitation was started and charged
   Wet Compound A and heated the contents to 31-36° C. and held the contents for 15 to 17 hours. Contents cooled to 25±5° C. and were the filtered through the centrifuge and rinsed with polish filtered
57 kg 1:1 (v/v) Acetonitrile/Water solution and
96 kg Acetonitrile.
   Withdrew a representative sample from the reactor.
      Analysis: HPLC: 98.5%
         Chiral: 97.2%

TABLE 9

| Batch | | | | | |
|---|---|---|---|---|---|
| Number | Yield (kg) | Yield (%) | HPLC Purity | Chiral Purity | KF |
| 6.1 | 7.75 | 43% | 98.5% | 97.2% | 1.8% |

### Example 7. Crystallization of Amorphous Compound A (Step (V))

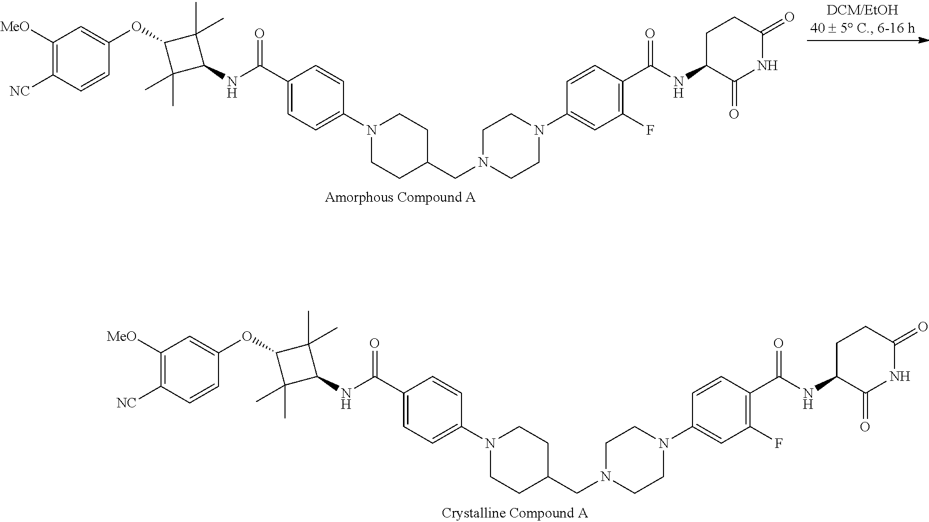 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N ; COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

TABLE 10
List of Materials for Example 7

| | | | | | Actual | |
|---|---|---|---|---|---|---|
| Reagents/Materials | MW | Eqs. | Moles | Density | Amount | |
| Compound A | 807.97 | 1.0 | 9.7 | — | 7.8 | kg |
| (amorphous) | | | | | | |
| Compound A | 807.97 | 0.005mass | — | — | 0.039 | kg |
| Seed Crystals | | | | | | |
| Ethanol (EtOH), | 46.07 | 40v | — | 0.82 | 246 | kg |
| absolute, 200 proof | | | | | | |

Procedure: To a 400 L reactor charged through a polish filter the contents of Compound A in DCM and polish filtered
5 kg DCM, started agitation and warned the contents to 31 to 37° C. Maintaining 31 to 37° C., over at least 30 minutes, through a polish filter, charged
92 kg Ethanol and then warmed then contents to 40±5° C. To the reactor, charged
   Compound A Seed Crystals and agitated for 15 to 30 minutes. With a slow stream of nitrogen, atmospherically distilled the contents until ˜117 L remained in the reactor.
      Withdrew a representative sample from the reactor.
      Analysis: DCM: 2.7%
   To the reactor charged
92 kg Ethanol and atmospherically distilled the contents until DCM was <2%, project chemist to determine. After completion, the contents were agitated for at least 1 hour at 40±5° C. and then slowly cooled the contents to 20±5° C. over at least 2 hours. At 20±5° C. the contents agitated for 14 to 16 hours in the reactor. Compound A was then filtered and rinsed with polish filtered
62 kg Ethanol. The Wet Compound A was then dried at ≤45° C.

TABLE 11
Production Results for Example 7

| Batch | | | | | |
|---|---|---|---|---|---|
| Number | Yield (kg) | Yield (%) | HPLC Purity | Chiral Purity | KF |
| 7.1 | 7.35 | 94.2 | 99.0% | 97.4% | 0.26% |

### Example 8. Synthesis of Intermediate I-3 (Step (I″))

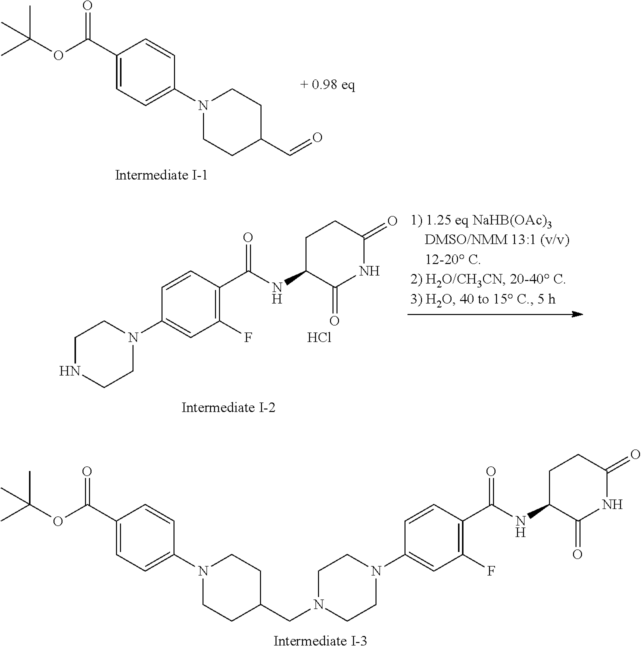 [SMILES: CC(C)(C)OC(=O)c1ccc(N2CCC(C=O)CC2)cc1 ; O=C1CC[C@H](NC(=O)c2ccc(N3CCNCC3)cc2F)C(=O)N1 ; Cl ; CC(C)(C)OC(=O)c1ccc(N2CCC(CN3CCN(c4ccc(C(=O)N[C@H]5CCC(=O)NC5=O)c(F)c4)CC3)CC2)cc1]

### Process Description:

1. Charge the reactor with Intermediate I-1 (24.0 kg, 82.9 mol), 0.98 eq. Intermediate I-2 (30.05 kg, 81.0 mol), DMSO (378 kg, 344 L) and 3.1 eq. N-Methylmorpholine (25.92 kg, 28 L, 256.3 mol).

2. Start agitation at 18-25° C. and continue stirring for 5-10 min until a clear solution is obtained.

3. Take a sample of the solution as IPC (HPLC): P0.

4. Cool the reaction mixture to 10-20° C.

5. Add 1.25 eq. sodium triacetoxyborohydride, STAB (21.98 kg, 103.7 mol) to the reaction mixture as a solid in 5 separate portions at 10-20° C. Wait 5-10 min after every addition.

6. Continue agitation of the reaction mixture at 10-20° C. Take first IPC sample after approx. 3 hours. If IPC criterion is not met continue agitation at 10-20° C. for max. 2 hours.

7. Add water (12 kg, 12 L) to the reaction mixture within approx. 20 min at 12-23° C.

8. Add Acetonitrile (309 kg, 396 L) to the reaction mixture within approx. 20 min at 12-23° C.

9. Warm the mixture to 35-40° C.

10. Add Intermediate I-3 seeding crystals (38 g) suspended in Acetonitrile to the solution.

11. Add water (64 kg, 64 L) to the solution within 20-30 min at 35-40° C.: precipitation.

12. Cool the slurry to 28-38° C.

13. Continue agitation for min. 3 hours at 28-38° C.

14. Cool the slurry stepwise and within 2 hours to 10-20° C.

15. Continue agitation for min. 2 hours at 10-20° C.

16. Collect the precipitate by filtration at 15-25° C.

17. Rinse the filter cake with an Acetonitrile/water mixture (approx. 319 L, 10:1 (v/v)) at 15-23° C. Combine rinse solution with the mother liquor.

18. Rinse the filter cake with an Acetonitrile/water mixture (approx. 160 L, 1:1 (v/v)) at 15-23° C. Combine rinse solution with the mother liquor.

19. Rinse the filter cake with an Acetonitrile/water mixture (approx. 80 L, 10:1 (v/v)) at 15-23° C. Combine rinse solution with the mother liquor.

20. Dry the product under vacuum at max. 45° C. until≤10.0 wt % water.

### Production

TABLE 12
Production Results for the Synthesis of Intermediate I-3.

| | Input | | | | | | | | | |
|---|---|---|---|---|---|---|---|---|---|---|
| | kg | Intermediate I-1 | | | Output | | | Yield | | |
| Batch | total | Intermediate I-2 | kg | assay | kg | assay | HI | as is | corr. | Comments |
| 9a | 2.50 | Intermediate I-1 | 2.50 | | 4.86 | 96.4% | 99.7% | 93% | 89% | 3 |
| | 3.13 | Intermediate I-2 | 3.13 | | | | | | | |
| 9b | 24.00 | Intermediate I-2 | 24.00 | | 35.17 | 90.4% | 99.7% | 70% | 63% | 3a |
| 9c | 30.05 | Intermediate I-2 | | | 16.44 | 68.7% | 99.8% | 33% | 22% | |
| | | Intermediate I-2 | | | | | | | | |
| 9d | 24.00 | Intermediate I-1 | 24.00 | | 40.70 | 92.7% | 99.7% | 81% | 75% | 3b |
| | 30.05 | Intermediate I-2 | 9.56 | | | | | | | |
| | | Intermediate I-2 | 20.49 | | | | | | | |
| 9e | | | | | 5.27 | 74.3% | 99.6% | 10% | 8% | |
| | 50.50 | | | | 97.17 | | | 95% | 86% | |

Comments:

| | |
|---|---|
| 3 | Final water content in isolated product: 2.5% wt./chiral purity 99.6% (HPLC) |
| 3a | Final water content in isolated product: 4.2% wt./chiral purity 99.6% (HPLC) |
| 3b | Final water content in isolated product: 5.7% wt./chiral purity 99.6% (HPLC) |

### Analytical Results

TABLE 13
Analytical Results for Batch 9a

| Attribute | Result |
|---|---|
| Color and Appearance | white solid |
| Identity (¹H NMR) | consistent |
| Assay (% w/w; ¹H NMR) | 96.4% |
| Purity (HPLC, % area) | 99.7% |
| Related substances (HPLC, % area) | |
| Specified impurities. | |
| Compound B (RRT 1.08) | N.D. |
| Intermediate I-2 (RRT 0.28) | N.D. |
| Individual unspecified impurities | 0.06% (RRT 0.518) |
| | 0.07% (RRT 0.522) |
| | 0.13% (RRT 1.344) |
| Total impurities | 0.3% |
| Chiral purity (HPLC, % area) | 99.6% |
| Water content (K.F.-Titration, % w/w) | 2.5% |

TABLE 14
Analytical Results for Batch 9b

| Attribute | Result |
|---|---|
| Color and Appearance | White to off-white solid |
| Identity (¹H-NMR) | Consistent with structure |
| Assay (% w/w; ¹H-NMR) | 90.4%-w/w |
| Purity (HPLC, % area) | 99.7% |
| Related substances (HPLC, % area) | |
| specified impurities | |
| Compound B (RRT 1.08) | N.D. |
| Intermediate I-2 (RRT 0.28) | N.D. |
| Individual unspecified | 0.05% (RRT 0.45) |
| impurities | 0.07% (RRT 0.52) |
| | 0.11% (RRT 0.53) |
| | 0.05% (RRT 1.05) |
| Total impurities | 0.28% |
| Chiral purity (HPLC, % area) | 99.6% |
| Water content (K.F.-Titration, %-w/w) | 4.2% w/w |

TABLE 15
Analytical Results for Batch 9c

| Attribute | Result |
|---|---|
| Color and Appearance | White to off-white solid |
| Identity (¹H-NMR) | Consistent with structure |
| Assay (% w/w; ¹H-NMR) | 68.7% |
| Purity (HPLC, % area) | 99.8% |
| Related substances (HPLC, % area) | |
| specified impurities | |
| Compound B (RRT 1.08) | Not detected |
| Intermediate I-2 (RRT 0.28) | <LOQ |
| Individual unspecified impurities | 0.05% (RRT 0.45) |
| | 0.07% (RRT 0.52) |
| | 0.13% (RRT 0.53) |
| Total impurities | 0.25% |
| Chiral purity (HPLC, % area) | 99.5% |
| Water content (K.F.-Titration, %-w/w) | 2.7% |

TABLE 16
Analytical Results for Batch 9d

| Attribute | Result | |
|---|---|---|
| Color and Appearance | White to off-white solid | |
| Identity (¹H-NMR) | Consistent with structure | |
| Assay (% w/w; ¹H-NMR) | 92.7%-w/w | |
| Purity (HPLC, % area) | 99.7% | |
| Related substances (HPLC, % area) | | |
| specified impurities | | |
| Compound B (RRT 1.08) | N.D. | |
| Intermediate I-2 (RRT 0.28) | N.D. | |
| Individual unspecified impurities | 0.05% | (RRT 0.35) |
| | 0.08% | (RRT 0.515) |
| | 0.08% | (RRT 0.519) |
| | 0.06% | (RRT 1.05) |
| Total impurities | 0.27% | |
| Chiral purity (HPLC, % area) | 99.6% | |
| Water content (K.F.-Titration, %-w/w) | 5.7% | w/w |

TABLE 17
Analytical Results for Batch 9e

| Attribute | Result | |
|---|---|---|
| Color and Appearance | White to off-white solid | |
| Identity (¹H-NMR) | Consistent with structure | |
| Assay (% w/w; ¹H-NMR) | 74.3% | |
| Purity (HPLC, % area) | 99.6% | |
| Related substances (HPLC, % area) | | |
| specified impurities | | |
| Compound B (RRT 1.08) | N.D. | |
| Intermediate I-2 (RRT 0.28) | N.D. | |
| Individual unspecified impurities | 0.05% | (RRT 0.35) |
| | 0.08% | (RRT 0.525) |
| | 0.13% | (RRT 0.530) |
| | 0.05% | (RRT 0.94) |
| | 0.07% | (RRT 1.05) |

| | |
|---|---|
| Total impurities | 0.38% |
| Chiral purity (HPLC, % area) | 99.3% |
| Water content (K.F.-Titration, %-w/w) | 8.4% |

### Example 9. Synthesis of Intermediate I-4 (Step (II″))

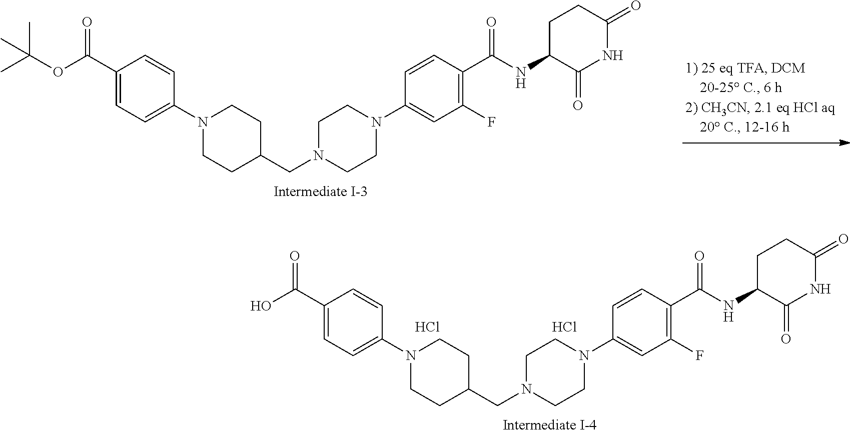 [SMILES: CC(C)(C)OC(=O)c1ccc(N2CCC(CN3CCN(c4ccc(C(=O)N[C@H]5CCC(=O)NC5=O)c(F)c4)CC3)CC2)cc1 ; O=C1CC[C@H](NC(=O)c2ccc(N3CCN(CC4CCN(c5ccc(C(=O)O)cc5)CC4)CC3)cc2F)C(=O)N1 ; Cl ; Cl]

### Process Description:

1. Charge the reactor with Intermediate I-3 (29.0 kg, 47.7 mol) and Dichloromethane, DCM (230.6 kg, 174 L).

2. Start agitation and adjust temperature to 20-25° C.

3. Add 24.6 eq Trifluoroacetic acid (134 kg, 1171 mol, 89.6 L) to the mixture over min. 15 min at 20-25° C.
During the addition the reaction mixture changes from a thick slurry to a clear solution.
Even though a significant amount of gas (isobutene) is released no foaming was observed on lab. scale.

4. Continue agitation of the reaction mixture at 15-25° C. Take first IPC sample after approx. 20 hours.

5. In-Process Control: Determine HPLC chromatogram of the reaction mixture.

$$\text{Criterion:} \frac{area \% ( Intermediate I - 3 )}{area \% ( Intermediate I - 3 ) + area \% ( Intermediate I - 4 )} \times 100 \% \leq 0.5 \%$$

6. Add Acetonitrile (477 kg, 612 L) to the solution over min. 10 min at 15-25° C. The reaction mixture remains a clear solution.

7. Add Intermediate I-4 seeding crystals (29 g) suspended in Acetonitrile to the solution.

8. Add 2.05 eq 32% hydrochloric acid (11.1 kg, 97.8 mol, 9.6 L) to the solution within 10 min at 15-25° C.: precipitation.

9. Continue agitation for 6-16 hours at 10-25° C.

10. Collect the precipitate by filtration at 15-25° C.

11. Rinse the filter cake in portions with Acetonitrile (271 kg, 348 L.)

12. Start drying of the filter cake in a flow of nitrogen for min. 30 min.

13. In-Process Control: Check Intermediate I-4 content of combined mother liquor/rinse solutions before disposal by HPLC.

14. Dry the product under vacuum at max. 45° C. until ≥96 wt % by QNMR (>99.7% by HPLC, >99.5% chiral purity, water content by KF: <0.5% w/w).

### Production

TABLE 18
Production Results for the Synthesis of Intermediate I-4.

| | Input | | | | | | | | | |
|---|---|---|---|---|---|---|---|---|---|---|
| | kg | Intermediate | | | Output | | | Yield | | |
| Batch | total | I-3 | kg | assay | kg | assay | HI | as is | corr. | Comments |
| 10a | 4.60 | 9a | 4.60 | 96.4% | 4.34 | 93.3% | 99.6% | 98% | 94% | 2, 3 |
| 10b | 29.00 | 9b | 29.00 | 90.4% | 27.92 | 96.7% | 99.7% | 94% | 100% | 2a, 3a |
| 10c | 29.00 | 9d | 29.00 | 92.7% | 28.36 | 96.8% | 99.9% | 95% | 99% | 2b, 3b |
| 10d | 34.50 | 9b | 5.11 | 90.4% | 28.33 | 96.7% | 99.8% | 80% | 97% | 2c, 3c |
| | | 9c | 16.26 | 68.7% | | | | | | |
| | | 9d | 11.25 | 92.7% | | | | | | |
| | | 9e | 1.88 | 74.3% | | | | | | |
| | 97.10 | | | | 88.95 | | | 92% | 98% | |

Comments:

| | |
|---|---|
| 2 | conversion Intermediate I-3 to Intermediate I-4 = 99.7% (HPLC) |
| | assay of dried filter cake = 92.1% wt. (QNMR) |
| 2a | conversion Intermediate I-3 to Intermediate I-4 = 99.7% (HPLC) |
| | assay of dried filter cake = 98.0% wt. (QNMR) |
| 2b | conversion Intermediate I-3 to Intermediate I-4 = 99.7% (HPLC) |
| | assay of dried filter cake = 96.2% wt. (QNMR) |
| 2c | conversion Intermediate I-3 to Intermediate I-4 = 99.8% (HPLC) |
| | assay of dried filter cake = 99.8% wt. (QNMR) |
| 3 | Water (K.F.): 0.7% wt./chiral purity 99.7% (HPLC)/assay |
| | TFA (¹⁹F NMR): 0.35% wt./Chloride: 11.8% wt. |
| 3a | Water (K.F.): 0.4% wt./chiral purity 99.6% (HPLC)/assay |
| | TFA (¹⁹F NMR): 0.32% wt./Chloride: not available |
| 3b | Water (K.F.): 0.3% wt./chiral purity 99.8% (HPLC)/assay |
| | TFA (¹⁹F NMR): 0.16% wt./Chloride: 11.6% wt. |
| 3c | Water (K.F.): 0.5% wt./chiral purity 99.6% (HPLC)/assay |
| | TFA (¹⁹F NMR): 0.26% wt./Chloride: 9.8% wt. |

### Analytical Results

TABLE 19
Analytical Results for Batch 10a

| Attribute | Result |
|---|---|
| Color and Appearance | white solid |
| Identity (¹H NMR) | consistent |
| Assay (qNMR, % w/w) | 93.3% |
| TFA (¹⁹F NMR, % w/w) | 0.35% |
| Purity (HPLC, % area) | 99.6% |
| Related substances (HPLC, % area) | |
| Specified impurities. | |
| Intermediate I-2 (RRT 0.84) | N.D. |
| Intermediate I-3 (RRT 3.03) | N.D. |
| Individual unspecified impurities | 0.15% (RRT 0.731) |
| | 0.12% (RRT 1.456) |
| | 0.13% (RRT 3.066) |
| Total impurities | 0.3% |
| Chiral purity (HPLC, % area) | 99.7% |
| Water content (K.F.-Titration, % w/w) | 0.7% |

TABLE 20
Analytical Results for Batch 10b

| Attribute | Result | |
|---|---|---|
| Color and Appearance | White to off-white solid | |
| Identity (¹H NMR) | Consistent with structure | |
| Assay (qNMR, %-w/w) | 96.7% | w/w |
| TFA (¹⁹F NMR, %-w/w) | 0.32% | |
| Purity (HPLC, % area) | 99.7% | |
| Related substances (HPLC, % area) | | |
| specified impurities | | |
| Intermediate I-2 (RRT 0.84) | <LOQ | |
| Intermediate I-3 (RRT 3.03) | 0.12% | |
| Individual unspecified impurities | 0.15% | (RRT 0.74) |
| Total impurities | 0.27% | |
| Chiral purity (HPLC, area %) | 99.6% | |
| Water (% wt.) | 0.37% | wt. |

TABLE 21
Analytical Results for Batch 10c

| Attribute | Result | |
|---|---|---|
| Color and Appearance | Off-white solid | |
| Identity (¹H NMR) | Consistent with structure | |
| Assay (qNMR, %-w/w) | 96.8% | w/w |
| TFA (¹⁹F NMR, %-w/w) | 0.16% | |
| Purity (HPLC, % area) | 99.9% | |
| Related substances (HPLC, % area) | | |
| specified impurities | | |
| Intermediate I-2 (RRT 0.84) | <LOQ | |
| Intermediate I-3 (RRT 3.03) | <LOQ | |
| Individual unspecified impurities | 0.14% | (RRT 0.73) |
| Total impurities | 0.14% | |
| Chiral purity (HPLC, area %) | 99.8% | |
| Water (% wt.) | 0.3% | wt. |

TABLE 22
Analytical Results for Batch 10d

| Attribute | Result | |
|---|---|---|
| Color and Appearance | Off-white solid | |
| Identity (¹H NMR) | Consistent with structure | |
| Assay (qNMR, %-w/w) | 96.7% | w/w |
| TFA (¹⁹F NMR, %-w/w) | 0.26% | |
| Purity (HPLC, % area) | 99.8% | |
| Related substances (HPLC, % area) | | |
| specified impurities | | |
| Intermediate I-2 (RRT 0.84) | <LOQ | |
| Intermediate I-3 (RRT 3.03) | <LOQ | |
| Individual unspecified impurities | 0.10% | (RRT 0.74) |
| | 0.12% | (RRT 0.96) |
| Total impurities | 0.22% | |
| Chiral purity (HPLC, area %) | 99.6% | |
| Water (% wt.) | 0.5% | wt. |

### Example 10. Synthesis of Compound A-Step (III″)

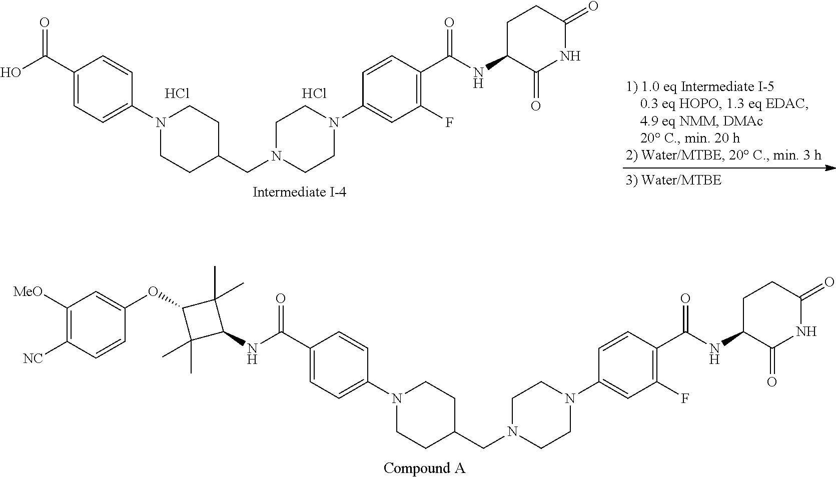 [SMILES: O=C1CC[C@H](NC(=O)c2ccc(N3CCN(CC4CCN(c5ccc(C(=O)O)cc5)CC4)CC3)cc2F)C(=O)N1 ; Cl ; Cl ; COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

### Process Description

1. Charge the reactor with Intermediate I-4 (4.04 kg, 6.87 mol), 1.0 eq. Intermediate I-5 (2.14 kg, 6.89 mol), 0.3 eq HOPO (0.23 kg, 2.07 mol) and DMAc (18.9 kg, 20 L).

2. Start agitation at 18-25° C.

3. Add 4.9 eq NMM (3.43 kg, 33.95 mol, 3.7 L) to the reaction mixture over min. 10 min at ≤ 25° C.

4. Add 1.3 eq EDAC (1.70 kg, 8.89 mol) as a solid in two portions over min. 15 min at ≤25° C.

5. Continue agitation of the reaction mixture at 15-25° C. Take first IPC sample after approx. 16 h.

6. In-Process Control: Determine HPLC chromatogram of the reaction mixture. Criterion: Conversion Intermediate I-4 to Compound A (crude) ≥98.8% (HPLC)

7. Charge a second reactor with water (121 kg, 121 L) at 15-25° C.

8. Add Compound A (crude) seeding crystals (6 g) suspended in water.

9. Add the reaction mixture from the first reactor to the second reactor over 30 min at 15-30° C.: precipitation.

10. Rinse the first reactor and the line with water (5 kg, 5 L).

11. Continue agitation for approx. 60 min at 15-25° C.

12. Add MTBE (15.0 kg, 20 L) to the slurry within 30-45 min at 15-25° C.

13. Continue agitation for min. 2 hours at 15-25° C.

14. Collect the precipitate by filtration.

15. Rinse the filter cake in portions with water (81 kg, 81 L) at ambient temperature. Combine rinse solution with the mother liquor.

16. Rinse the filter cake in portions with MTBE (30 kg, 40 L at ambient temperature.

Combine rinse solution with the mother liquor.

17. In-Process Control: Check Compound A (crude) content of combined mother liquor/rinse solutions by HPLC before disposal.

18. Dry the product under vacuum at max. 45° C. until ≥92 wt % (QNMR).

### Production

TABLE 23
Production Results for the Synthesis of Compound A.

| | Input | | | | | | | | |
|---|---|---|---|---|---|---|---|---|---|
| | kg | Intermediate | | | Output | | | Yield | |
| Batch | total | I-4 | kg | assay | kg | assay | HI | as is | corr. |
| 11a | 4.04 | 10a | 4.04 | 93.1% | 5.48 | 92.9% | 98.5% | 99% | 99%* |
| | 4.04 | | | | 5.48 | | | 99% | 99% |

*conversion Intermediate I-4 to Compound A (crude) was 99.9% vs. NLT 99.8% specified.

### Analytical Results

TABLE 24
Analytical Results for Batch 11a

| Attribute | Result |
|---|---|
| Color and Appearance | white solid with lumps |
| Identity (¹H NMR) | consistent |
| Assay (qNMR, % w/w) | 92.9% |
| Purity (HPLC, % area) | 98.5% |
| Related substances (HPLC, % area) | |
| Specified impurities | |
| Mixture of Compound E & | N.D. |
| Compound F (RRT 0.60) | |
| Individual unspecified impurities | 0.11% (RRT 0.360) |
| | 0.19% (RRT 0.594) |
| | 0.15% (RRT 0.652) |
| | 0.10% (RRT 0.739) |
| | 0.14% (RRT 1.045) |
| | 0.11% (RRT 1.075) |
| | 0.11% (RRT 1.315) |
| Total impurities | 1.5% |
| Chiral purity (HPLC, % area) | 98.9% |
| Water content (K.F.-Titration, % w/w) | 1.6% |

### Example 11. Synthesis of Compound A-Step (III′″)

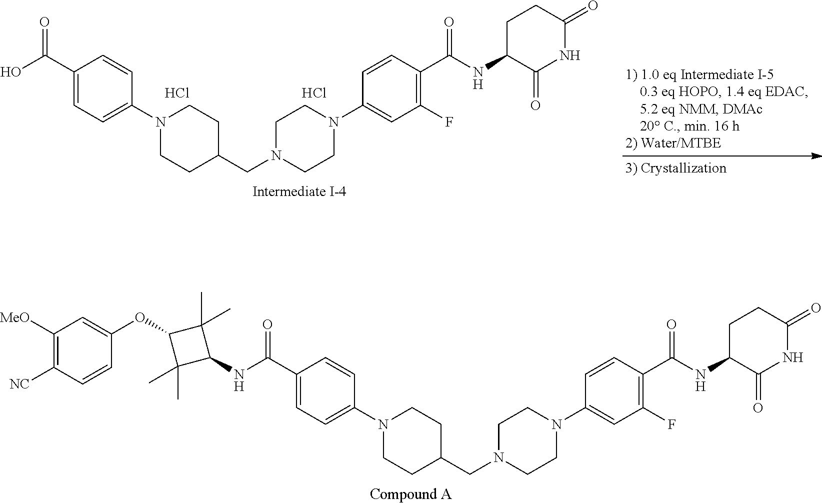 [SMILES: O=C1CC[C@H](NC(=O)c2ccc(N3CCN(CC4CCN(c5ccc(C(=O)O)cc5)CC4)CC3)cc2F)C(=O)N1 ; Cl ; Cl ; COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

### Process Description:

The following process description is based on batch 12e (scale up batch):

1. Charge the reactor with Intermediate I-4 (21.0 kg, 33.6 mol), 1.1 eq Intermediate I-5 (11.1 kg, 35.8 mol), 0.3 eq 2-hydroxypyridine 1-oxide, HOPO (1.20 kg, 10.8 mol) and Dimethylacetamide, DMAc (98 kg, 105 L).

2. Start agitation.

3. Dose 5.2 eq N-Methylmorpholine, NMM (17.9 kg, 176.5 mol, 19.4 L) to the mixture over min. 10 min at ≤25° C.

During the addition the reaction mixture changes from a thick slurry to a clear solution and back to a stirrable suspension.

4. Adjust the temperature of the slurry to 15-25° C.

5. Add 1.4 eq EDAC*HCl (8.9 kg, 46.2 mol) to the mixture as a solid in two portions within min.
15 min at 15-25° C.

6. Continue agitation of the reaction mixture at 15-25° C. Take first IPC sample after approx. 16 h.

7. In-Process Control: Determine HPLC chromatogram of the reaction mixture.

$$\text{Criterion:} \frac{area \% ( Compound A )}{area \% ( Compound A ) + area \% ( Intermediate I - 4 )} \times 100 \% \geq 99.8 \%$$

8. Charge a second reactor with water (630 kg, 630 L) at 15-25° C.

9. Add Compound A (crude) seeding crystals (32 g) suspended in water to the solution.

10. Add the reaction mixture from the first reactor to the second reactor over min. 30 min at 15-30° C.

11. Rinse the transfer line with water (26 kg, 26 L) to the second reactor.

12. Continue agitation of the mixture in the second reactor for approx. 60 min at 15-25° C.

13. Add MTBE (78 kg, 105 L) to the suspension at 15-25° C.

14. Continue agitation for min. 2 hours at 15-25° C.

15. Stop agitation and allow the phases to separate.

16. Remove the clear mother liquor (bottom) from the reactor without agitation until the solution becomes turbid.

17. Filter the remaining slurry, if possible, in one filling of the filter unit.

18. Rinse the filter cake with water (420 kg, 420 L) and MTBE (155 kg, 210 L).

19. In-Process Control: Check Compound A (crude) content of the combined filtrates by

HPLC before disposal.

20. Dry the product under vacuum at max. 45° C. until a manageable solid is obtained . . .

21. Record the weight of the damp filter cake and determine the assay of Compound A (crude) by NMR, water content by K. F., purity and chiral purity by HPLC.

22. Calculate the theoretical weight of Compound A (crude): m (crude Compound A).

$$m ( crude Compound A ) = \frac{m ( wet cake ) \times assay ( crude Compound A )}{90 \%}$$

23. Charge the crystallization reactor with polish filtered ethanol (20 L/kg×m (crude Compound A)) at 15-25° C.

24. Charge another reactor with the damp product from step 20.

25. Add DCM (17.4 1/kg×m (crude Compound A)) to the product.

26. Stir the suspension for approx. 30-60 min at 18-25° C. until a clear solution develops.

27. Stop agitation and allow the layers to separate for min. 2 hours.
Two clear layers are expected.

28. Transfer the bottom, organic layer through a particle filter to the crystallization reactor.

29. Drain the top aqueous layers from the reactor separately and check Compound A content by HPLC before disposal.

30. Add Compound A seeding crystals (30 g) suspended in EtOH to the solution.

31. Rinse the line with DCM (1 L/kg×m (crude Compound A)).

32. Concentrate the solution to approx. 20 L/kg×m (crude Compound A) by distillation under reduced pressure at max. 45° C.: precipitation.

33. Add to the crystallization reactor via the particle filter EtOH (5 L/kg×m (crude Compound A).)

34. Concentrate the slurry to approx. 20 L/kg×m (crude Compound A) by distillation under vacuum at max. 45° C.

35. Add to the crystallization reactor via the particle filter EtOH (5 L/kg×m (crude Compound A).)

36. Concentrate the slurry to approx. 20 L/kg×m (crude Compound A) by distillation under vacuum at max. 45° C.

37. Purge the reactor with nitrogen and cool the slurry to 18-25° C. over min. 60 min.

38. Continue agitation for min. 8 hours at 18-25° C.

39. Collect the precipitate by filtration.

40. Rinse the filter cake in portions with EtOH via the particle filter and reactor (4 L/kg×m (crude Compound A).)

41. In-Process Control: Check Compound A content of combined mother liquor/rinse solutions by HPLC before disposal.

42. Dry the product under vacuum at max. 45° C. to provide crystalline Compound A (>99% HPLC purity, >98.5% chiral purity and >97% assay by HPLC).

### Example 12. Synthesis of Compound A-Sten (III″ ″″)

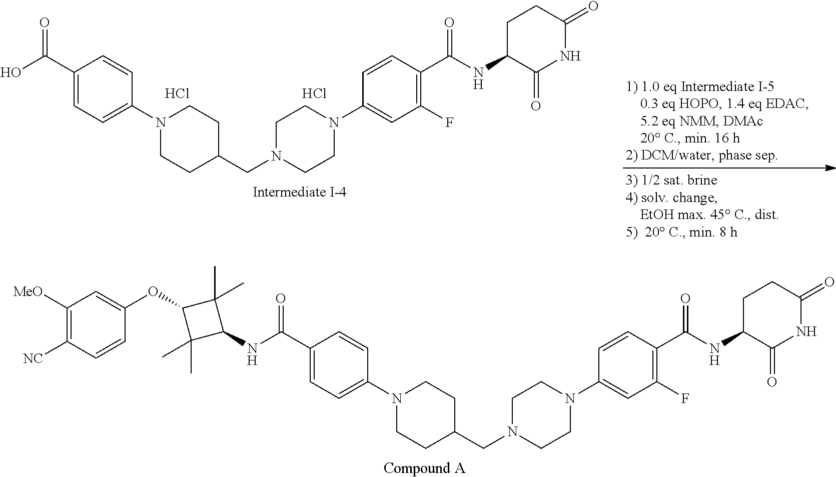 [SMILES: O=C1CC[C@H](NC(=O)c2ccc(N3CCN(CC4CCN(c5ccc(C(=O)O)cc5)CC4)CC3)cc2F)C(=O)N1 ; Cl ; Cl ; COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

The following process description is based on batch 12f (scale up batch):

1. Charge the reactor with Intermediate I-4 (20.3 kg, 32.4 mol), 1.1 eq Intermediate I-5 (10.7 kg, 34.6 mol), 0.3 eq 2-hydroxypyridine 1-oxide, HOPO (1.16 kg, 10.4 mol) and Dimethylacetamide, DMAc (93 kg, 101 L).

2. Start agitation.

3. Dose 5.3 eq N-Methylmorpholine, NMM (17.2 kg, 170.2 mol, 18.7 L) to the mixture over min. 10 min at ≤25° C.

During the addition the reaction mixture changes from a thick slurry to a clear solution and back to a stirrable suspension.

4. Adjust the temperature of the slurry to 15-25° C.

5. Add 1.4 eq EDAC*HCl (8.6 kg, 44.6 mol) to the mixture as a solid in two portions within min.

15 min at 15-25° C.

6. Continue agitation of the reaction mixture at 15-25° C. Take first IPC sample after approx. 16 h.

7. In-Process Control: Determine HPLC chromatogram of the reaction mixture.

$$\text{Criterion:} \frac{area \% ( Compound A )}{area \% ( Compound A ) + area \% ( Intermediate I - 4 )} \times 100 \% \geq 99.8 \%$$

8. Add Dichloromethane, DCM (537 kg, 420 L) to the slurry at 15-25° C.

9. Add Water (405 kg, 405 L) to the slurry at 15-25° C.

10. Stir the reaction mixture vigorously for a minimum of 20 minutes at 15-25° C.

11. Allow layers to separate for a minimum of 30 minutes.

12. Drain both layers from the reactor separately.

13. In-Process Control: Check Compound A content of the aqueous layer by HPLC before disposal.

14. Charge the reactor with the organic layer (bottom).

15. Add half sat. brine (13%) (509 kg, 405 L) to the reactor at 15-25° C.

16. Stir the reaction mixture vigorously for a minimum of 20 minutes at 15-25° C.

17. Allow layers to separate for 30 minutes.

18. Drain both layers from the reactor separately.

19. In-Process Control: Check Compound A content of the aqueous layer by HPLC before disposal.

20. Charge the (second) crystallization reactor with the organic layer (bottom) through a particle filter.

21. Rinse the line with DCM (27 kg, 20 L).

22. Add to the crystallization reactor via the particle filter EtOH (460 kg, 582 L) at 15-25° C.

23. Add Compound A seeding crystals (26 g) suspended in EtOH to the solution.

24. Concentrate the solution to approx. 582 L by distillation under reduced pressure at max. 45° C.: precipitation.

25. Add to the crystallization reactor via the particle filter EtOH (115 kg, 146 L) at max. 45° C.

26. Concentrate the solution to approx. 582 L by distillation under reduced pressure at max. 45° C.

27. Add to the crystallization reactor via the particle filter EtOH (115 kg, 146 L) at max. 45° C.

28. Concentrate the solution to approx. 582 L by distillation under reduced pressure at max. 45° C.

29. Purge the reactor with nitrogen and cool the slurry to 18-25° C. over a minimum of 60 minutes.

30. Continue agitation for a minimum of 8 hours at 18-25° C.

31. Collect the precipitate by filtration.

32. Rinse the filter cake in portions with EtOH via the particle filter (92 kg, 116 L.)

33. In-Process Control: Check Compound A content of combined mother liquor/rinse solutions by HPLC before disposal.

34. Dry the product under vacuum at max. 45° C. to provide Crystalline Compound A (>99% HPLC purity, >98.5% chiral purity and >97% assay by HPLC).

### Production

TABLE 25
Production Results for the Synthesis of Compound A.

| | Input | | | | | | | | | |
|---|---|---|---|---|---|---|---|---|---|---|
| | kg | Compound A | | | Output | | | Yield | | |
| Batch | total | (crude) | kg | assay | kg | assay | HI | as is | corr. | Comments |
| 12a | 5.24 | 11a | 5.24 | 92.9% | 4.58 | 98.9% | 99.5% | 87% | 93% | 3 |
| | kg | Intermediate | | | | | | | | |
| | total | I-4 | kg | assay | kg | assay | HI | as is | corr. | |
| 12b | 27.80 | 10b | 27.80 | 96.7% | 13.76 | 97.5% | 99.3% | 38% | 39% | 3a, 7a |
| 12c | | | | | 18.21 | 98.5% | 99.2% | 51% | 52% | 3b, 7b |
| 12d | 14.00 | 10c | 14.00 | 96.8% | 13.94 | 96.4% | 98.4% | 77% | 77% | 3c, 7c |
| 12e | 21.00 | 10c | 14.16 | 96.8% | 21.40 | 98.0% | 99.5% | 79% | 79% | 3d, 7d |
| | | 10d | 6.84 | 99.8% | | | | | | |
| 12f | 20.26 | 10d | 20.26 | 99.8% | 23.14 | 98.0% | 99.5% | 88% | 87% | 3e |
| | 88.30 | | | | 95.03 | | | | 85% | |

Comments:

| | |
|---|---|
| 3 | Compound A: Assay (HPLC): 98.9% wt., Purity (HPLC): 99.5 area %, Chiral purity |
| | (HPLC): 99.0%, Water (KF): 0.15% wt., RoI: <0.1% wt., EtOH: 555 ppm, MeCN: <41 |
| | ppm, DCM: 1118 ppm, MTBE: n.d., NMM: <100 ppm, DMAc: 112 ppm, DMSO: n.d., |
| | HOAc (GC): n.d., TFA (IC): <500 ppm |
| 3a | Compound A: Assay (HPLC): 97.5% wt., Purity (HPLC): 99.3 area %, Chiral purity |
| | (HPLC): 98.8%, Water (KF): 0.11% wt., RoI: <0.1% wt., EtOH: 2953 ppm, MeCN: |
| | n.d., DCM: 3769 ppm, MTBE: n.d., NMM: n.d., DMAc: n.d., DMSO: n.d., HOAc (GC): |
| | n.d., TFA (IC): <100 ppm |
| 3b | Compound A: Assay (HPLC): 98.5% wt., Purity (HPLC): 99.2 area %, Chiral purity |
| | (HPLC): 98.7%, Water (KF): 0.10% wt., RoI: <0.1% wt., EtOH: 1841 ppm, MeCN: |
| | n.d., DCM: 2392 ppm, MTBE: n.d., NMM: n.d., DMAc: <109 ppm, DMSO: n.d., HOAc |
| | (GC): n.d., TFA (IC): <100 ppm |
| 3c | Compound A: Assay (HPLC): 96.4% wt., Purity (HPLC): 98.4 area %, Chiral purity |
| | (HPLC): 98.7%, Water (KF): 0.49% wt., RoI: <0.1% wt., EtOH: 1604 ppm, MeCN: |
| | n.d., DCM: 779 ppm, MTBE: n.d., NMM: n.d., DMAc: 360 ppm, DMSO: n.d., HOAc |
| | (GC): n.d., TFA (IC): <100 ppm |
| 3d | Compound A: Assay (HPLC): 98.0% wt., Purity (HPLC): 99.5 area %, Chiral purity |
| | (HPLC): 98.6%, Water (KF): 0.21% wt., RoI: <0.1% wt., EtOH: 767 ppm, MeCN: n.d., |
| | DCM: n.d., MTBE: n.d., NMM: n.d., DMAc: 2462 ppm, DMSO: n.d., HOAc (GC): n.d., |
| | TFA (IC): <100 ppm |
| 3e | Compound A: Assay (HPLC): 98.0% wt., Purity (HPLC): 99.5 area %, Chiral purity |
| | (HPLC): 98.8%, Water (KF): 0.08% wt., RoI: <0.1% wt., EtOH: 2113 ppm, MeCN: |
| | n.d., DCM: n.d., MTBE: n.d., NMM: n.d., DMAc: 2209 ppm, DMSO: n.d., HOAc (GC): |
| | n.d., TFA (IC): <100 ppm |
| 7a | Compound A (crude): 15.67 kg, qNMR: 88% wt., purity: 98.8 area % (HPLC), chiral |
| | purity: 98.9% (HPLC) |
| 7b | Compound A (crude): 26.49 kg, qNMR: 65% wt., purity: 98.6 area % (HPLC), chiral |
| | purity: 98.8% (HPLC) |
| 7c | Compound A (crude): 29.19 kg, qNMR: 50% wt., purity: 97.3 area % (HPLC), Water |
| | (KF): 49.9% wt. |
| 7d | Compound A (crude): 35.56 kg, qNMR: 63% wt., purity: 97 area % (HPLC), chiral |
| | purity: 98.7% (HPLC), Water (KF): 18.6% wt. |

### Analytical Results

TABLE 26
Analytical Results for Batch 12a

| Attribute | Result | |
|---|---|---|
| Color and Appearance | White to off-white solid | |
| Identity | | |
| IR | Consistent to reference spectrum | |
| HPLC RT | Conforms to Reference | |
| ¹H-NMR | See spectrum | |
| ¹³C-NMR | See spectrum | |
| Assay (HPLC, %-w/w “as is”) | 98.9% | |
| Purity (HPLC, % area) | 99.5% | |
| Related substances (HPLC, % area) | | |
| specified impurities | | |
| RRT 0.35 (Intermediate I-4) | 0.08% | |
| RRT 0.55 | 0.05% | |
| RRT 0.60 (Mixture of | 0.16% | |
| Compound E & Compound F) | | |
| RRT 0.74 | 0.07% | |
| RRT 1.08 (Intermediate I-3) | <0.05% | |
| Individual unspecified impurities | <0.05% | (RRT 0.61) |
| (Report all ≥0.05% with RRT) | 0.08% | (RRT 1.05) |
| | 0.09% | (RRT 1.32) |
| Total impurities | 0.53% | |
| Chiral purity by HPLC | 99.0% | |
| Water content by Karl Fisher (% wt.) | 0.15% | |
| Residual solvents (GC-HS) | | |
| Ethanol (EtOH) | 555 | ppm |
| Acetonitrile (ACN) | <41 | ppm |
| Dichloromethane (DCM) | 1118 | ppm |
| Methyl tert-Butyl ether (MTBE) | Not detected | |
| n-Methylmorpholine | <100 | ppm |
| N,N-Dimethylacetamide (DMAc) | 112 | ppm |
| Dimethyl sulfoxide (DMSO) | Not detected | |
| Acetic acid content (GC) | Not detected | |
| DSC | 238° | C. |
| Particle size (D10, D50, D90) | d₁₀: 4.089 μm | |
| | d₅₀: 20.10 μm | |
| | d₉₀: 67.64 μm | |
| Elemental Impurities | | |
| (corresp. To USP <232>/<233>) | | |
| Arsenic (As) | <1 | ppm |
| Cadmium (Cd) | <0.5 | ppm |
| Mercury (Hg) | <1 | ppm |
| Lead (Pb) | <0.5 | ppm |
| Cobalt (Co) | <1 | ppm |
| Nickel (Ni) | <1 | ppm |
| Vanadium (V) | <1 | ppm |

TABLE 27
Analytical Results for Batch 12b

| Attribute | Result | |
|---|---|---|
| Color and Appearance | Off-white solid | |
| Identity | | |
| FT-IR | Consistent to reference spectrum | |
| HPLC Retention time | Conforms to reference standard | |
| Assay (HPLC, %-w/w) | | |
| Assay “as is” | 97.5% | |
| Assay “water and solvent free” | 98.2% | |
| Assay “corrected for chiral purity” | 96.3% | |
| Purity (HPLC, % area) | 99.3% | |
| Related substances (HPLC, % area) | | |
| Specified impurities | | |
| Intermediate I-4 (RRT 0.35) | <LOQ | |
| Compound C (RRT 0.54) | <LOQ | |
| Compound D (RRT 0.55) | <LOQ | |
| Compound E (RRT 0.580) | 0.06% | |
| Compound F (RRT 0.584) | 0.26% | |
| Compound G (RRT 0.74) | 0.05% | |
| RRT 1.05 | 0.07% | |
| Intermediate I-3 (RRT 1.08) | 0.05% | |
| Individual unspecified impurities | 0.07% | (RRT 0.55) |
| (Report all ≥0.05% with RRT) | 0.05% | (RRT 1.11) |
| | 0.05% | (RRT 1.20) |
| | 0.07% | (RRT 1.57) |
| Total impurities | 0.73% | |
| Chiral purity (HPLC, % area) | | |
| Compound A | 98.8% | |
| epi-Compound A | 1.2% | |
| Water content by Karl Fisher (%-w/w) | 0.11% | |
| Residue on Ignition | <0.1% | |
| DSC | 240° | C. |
| Particle Size Distribution | d₁₀: 4.49 | |
| | d₅₀: 24.6 | |
| | d₉₀: 111.2 | |
| Residual solvents (GC-HS) | | |
| Ethanol (EtOH) | 2953 | ppm |
| Acetonitrile (ACN) | Not detected | |
| Dichloromethane (DCM) | 3769 | ppm |
| Methyl tert-Butyl ether (MTBE) | Not detected | |
| N-Methylmorpholine | Not detected | |
| N,N-Dimethylacetamide (DMAc) | Not detected | |
| Dimethyl sulfoxide (DMSO) | Not detected | |
| Acetic acid content (GC) | Not detected | |
| Trifluoroacetic acid (TFA) content | <100 | ppm |
| Elemental Impurities | | |
| (corresp. to USP <232>/<233>) | | |
| Arsenic (As) | <1 | ppm |
| Cadmium (Cd) | <0.5 | ppm |
| Mercury (Hg) | <1 | ppm |
| Lead (Pb) | <0.5 | ppm |
| Cobalt (Co) | <1 | ppm |
| Nickel (Ni) | <1 | ppm |
| Vanadium (V) | <1 | ppm |

TABLE 28
Analytical Results for Batch 12c

| Attribute | Result | |
|---|---|---|
| Color and Appearance | White to off-white solid | |
| Identity | | |
| FT-IR | Consistent to reference spectrum | |
| HPLC Retention time | Conforms to reference standard | |
| Assay (HPLC, %-w/w) | | |
| Assay “as is” | 98.5% | |
| Assay “water and solvent free” | 99.0% | |
| Assay “corrected for chiral purity” | 97.2% | |
| Purity (HPLC, % area) | 99.2% | |
| Related substances (HPLC, % area) | | |
| specified impurities | | |
| Intermediate I-4 (RRT 0.35) | <LOQ | |
| Compound C (RRT 0.54) | <LOQ | |
| Compound D (RRT 0.55) | <LOQ | |
| Compound E (RRT 0.580) | 0.06% | |
| Compound F (RRT 0.584) | 0.29% | |
| Compound G (RRT 0.74) | 0.05% | |
| RRT 1.05 | 0.13% | |
| Intermediate I-3 (RRT 1.08) | 0.05% | |
| Individual unspecified impurities | 0.08% | (RRT 0.55) |
| (Report all ≤0.05% with RRT) | 0.06% | (RRT 1.11) |
| | 0.06% | (RRT 1.20) |
| | 0.07% | (RRT 1.57) |
| Total impurities | 0.85% | |
| Chiral purity (HPLC, % area) | | |
| Compound A | 98.7% | |
| epi-Compound A | 1.3% | |
| Water content by Karl Fisher (%-w/w) | 0.10% | |
| Residue on Ignition | <0.1% | |
| DSC | 241° | C. |
| Particle Size Distribution | d₁₀: 3.74 | |
| | d₅₀: 189 | |
| | d₉₀: 58.6 | |
| Residual solvents (GC-HS) | | |
| Ethanol (EtOH) | 1841 | ppm |
| Acetonitrile (ACN) | Not detected | |
| Dichloromethane (DCM) | 2392 | ppm |
| Methyl tert-Butyl ether (MTBE) | Not detected | |
| N-Methylmorpholine | Not detected | |
| N,N-Dimethylacetamide (DMAc) | <109 | ppm |
| Dimethyl sulfoxide (DMSO) | Not detected | |
| Acetic acid content (GC) | Not detected | |
| Trifluoroacetic acid (TFA) content | <100 | ppm |
| Elemental Impurities | | |
| (corresp. to USP <232>/<233>) | | |
| Arsenic (As) | <1 | ppm |
| Cadmium (Cd) | <0.5 | ppm |
| Mercury (Hg) | <1 | ppm |
| Lead (Pb) | <0.5 | ppm |
| Cobalt (Co) | <1 | ppm |
| Nickel (Ni) | <1 | ppm |
| Vanadium (V) | <1 | ppm |

TABLE 29
Analytical Results for Batch 12d

| Attribute | Result | |
|---|---|---|
| Color and Appearance | White to off-white solid | |
| Identity | | |
| FT-IR | Consistent to reference spectrum | |
| HPLC Retention time | Consistent to reference standard | |
| Assay (HPLC, %-w/w) | | |
| Assay “as is” | 96.4% | |
| Assay “water and solvent free” | 97.2% | |
| Assay “corrected for chiral purity” | 95.2% | |
| Purity (HPLC, % area) | 98.4% | |
| Related substances (HPLC, % area) | | |
| Specified impurities | | |
| Intermediate I-4 (RRT 0.35) | 0.94% | |
| Compound C (RRT 0.54) | 0.05% | |
| Compound D (RRT 0.55) | 0.06% | |
| Compound E (RRT 0.580) | 0.09% | |
| Compound F (RRT 0.584) | 0.29% | |
| Compound G (RRT 0.74) | 0.05% | |
| (RRT 1.05) | 0.08% | |
| Intermediate I-3 (RRT 1.08) | <LOQ | |
| Individual unspecified impurities | None detected | |
| (Report all ≥0.05% with RRT) | | |
| Total impurities | 1.6% | |
| Chiral purity (HPLC, % area) | | |
| Compound A | 98.7% | |
| epi-Compound A | 1.3% | |
| Water content by Karl Fisher (%-w/w) | 0.49% | |
| Residue on Ignition | <0.1% | w/w |
| DSC | 240° | C. |
| Particle Size Distribution | d₁₀: 6.736 μm | |
| | d₅₀: 25.11 μm | |
| | d₉₀: 79.67 μm | |
| Residual solvents (GC-HS) | | |
| Ethanol (EtOH) | 1604 | ppm |
| Acetonitrile (ACN) | Not detected | |
| Dichloromethane (DCM) | 779 | ppm |
| Methyl tert-Butyl ether (MTBE) | Not detected | |
| N-Methylmorpholine | Not detected | |
| N,N-Dimethylacetamide (DMAc) | 360 | ppm |
| Dimethyl sulfoxide (DMSO) | Not detected | |
| Acetic acid content (GC) | Not detected | |
| Trifluoroacetic acid (TFA) content | <100 | ppm |
| Elemental impurities | | |
| (corresp. to USP <232>/<233>) | | |
| Arsenic (As) | <1 | ppm |
| Cadmium (Cd) | <0.5 | ppm |
| Mercury (Hg) | <1 | ppm |
| Lead (Pb) | <0.5 | ppm |
| Cobalt (Co) | <1 | ppm |
| Nickel (Ni) | <1 | ppm |
| Vanadium (V) | <1 | ppm |

TABLE 30
Analytical Results for Batch 12e

| Attribute | Result | |
|---|---|---|
| Color and Appearance | White to off-white solid | |
| Identity | | |
| FT-IR | Consistent to reference spectrum | |
| HPLC Retention time | Conforms to reference standard | |
| Assay (HPLC, %-w/w) | | |
| Assay “as is” | 98.0% | |
| Assay “water and solvent free” | 98.5% | |
| Assay “corrected for chiral purity” | 96.6% | |
| Purity (HPLC, % area) | 99.5% | |
| Related substances (HPLC, % area) | | |
| specified impurities | | |
| Intermediate I-4 (RRT 0.35) | <LOQ | |
| Compound C (RRT 0.54) | <LOQ | |
| Compound D (RRT 0.55) | 0.05% | |
| Compound E (RRT 0.580) | 0.06% | |
| Compound F (RRT 0.584) | 0.22% | |
| Compound G (RRT 0.74) | 0.05% | |
| RRT 1.05 | 0.10% | |
| Intermediate I-3 (RRT 1.08) | <LOQ | |
| Individual unspecified Impurities | 0.05% | (RRT 1.20) |
| (Report all ≥0.05% with RRT) | | |
| Total Impurities | 0.53% | |
| Chiral purity (HPLC, % area) | | |
| Compound A | 98.6% | |
| epi-Compound A | 1.4% | |
| Water content by Karl Fisher (%-w/w) | 0.21% | |
| Residue on Ignition | <0.1% | w/w |
| DSC | 242° | C. |
| Particle Size Distribution | d₁₀: 15.80 μm | |
| | d₅₀: 83.34 μm | |
| | d₉₀: 171.8 μm | |
| Residual solvents (GC-HS) | | |
| Ethanol (EtOH) | 767 | ppm |
| Acetonitrile (ACN) | Not detected | |
| Dichloromethane (DCM) | Not detected | |
| Methyl tert-Butyl ether (MTBE) | Not detected | |
| N-Methylmorpholine | Not detected | |
| N,N-Dimethylacetamide (DMAc) | 2462 | ppm |
| Dimethyl sulfoxide (DMSO) | Not detected | |
| Acetic acid content (GC) | Not detected | |
| Trifluoroacetic acid (TFA) content | <100 | ppm |
| Elemental Impurities | | |
| (corresp. to USP <232>/<233>) | | |
| Arsenic (As) | <1 | ppm |
| Cadmium (Cd) | <0.5 | ppm |
| Mercury (Hg) | <1 | ppm |
| Lead (Pb) | <0.5 | ppm |
| Cobalt (Co) | <1 | ppm |
| Nickel (Ni) | <1 | ppm |
| Vanadium (V) | <1 | ppm |

TABLE 31
Analytical Results for Batch 12f

| Attribute | Result | |
|---|---|---|
| Color and Appearance | White to off-white solid | |
| Identity | | |
| FT-IR | Consistent to reference spectrum | |
| HPLC Retention time | Conforms to reference standard | |
| Assay (HPLC, %-w/w) | | |
| Assay “as is” | 98.0% | |
| Assay “water and solvent free” | 98.8% | |
| Assay “corrected and chiral purity” | 98.8% | |
| Purity (HPLC, % area) | 99.4% | |
| Related substances (HPLC, % area) | | |
| specified impurities | | |
| Intermediate I-4 | <LOQ | |
| Compound C | <LOQ | |
| Compound D | <LOQ | |
| Compound E + Compound F | <LOQ + 0.09% = 0.09% | |
| Compound G | 0.12% | |
| RRT 1.05 | 0.12% | |
| Intermediate I-3 | <LOQ | |
| Individual unspecified impurities | 0.06% | (RRT 0.55) |
| (Report all ≥0.05% with RRT) | 0.05% | (RRT 1.11) |
| | 0.06% | (RRT 1.20) |
| | 0.05% | (RRT 1.57) |
| Total impurities | 0.55% | |
| Chiral purity (HPLC, % area) | | |
| Compound A | 98.8% | |
| epi-Compound A | 1.2% | |
| Water content by Karl Fisher (%-w/w) | 0.08% | |
| Residue on Ignition | <0.1% | m/m |
| DSC | 242.6° | C. |
| Particle Size Distribution | d₁₀ = 7.534 μm | |
| | d₁₀ = 28.13 μm | |
| | d₁₀ = 63.85 μm | |
| Residual solvents (GC-HS) | | |
| Ethanol (EtOH) | 2113 | pp, |
| Acetonitrile (CAN) | Not detected | |
| Dichloromethane (DCM) | 3285 | ppm |
| N-Methylmorpholine | Not detected | |
| N,N-Dimethylacetamide (DMAc) | 2209 | ppm |
| Dimethyl sulfoxide (DMSO) | Not detected | |
| Acetic acid content (GC) | Not detected | |
| Trifluoroacetic acid (TFA) content | <100 | ppm |
| Elemental impurities | | |
| (corresp. To USP <232>/<233>) | | |
| Arsenic (As) | <1 | ppm |
| Cadmium (Cd) | <0.5 | ppm |
| Mercury (Hg) | <1 | ppm |
| Lead (Pb) | <0.5 | ppm |
| Cobalt (Co) | <1 | ppm |
| Nickel (Ni) | <1 | ppm |
| Vanadium (V) | <1 | ppm |

### EQUIVALENTS

It is to be understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### Enumerated Embodiments

The aspects of the present disclosure are further described with references to the following numbered embodiments:

1. A method comprising Step (III):
(III) reacting Intermediate I-4:

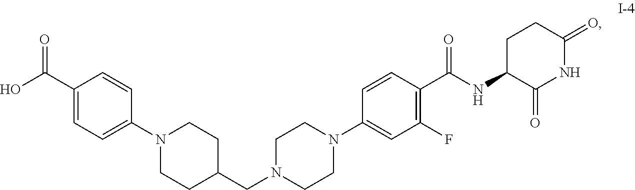 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

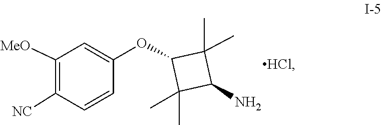 [SMILES: COc1cc(O[C@H]2C(C)(C)[C@H](N)C2(C)C)ccc1C#N]

in a reaction mixture, wherein the reaction mixture comprises a base, a coupling reagent, and an additive in a solvent to provide Compound A:

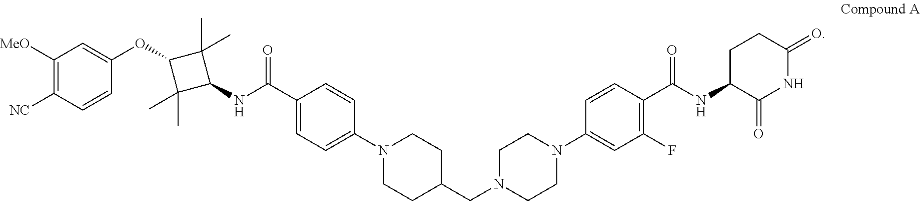 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

2. The method of Embodiment 1, wherein Compound A is provided in crude form.

3. The method of Embodiment 1, wherein Compound A is provided in crystalline form.

4. The method of any one of the preceding Embodiments, wherein the salt of Intermediate I-4 is a hydrochloride salt.

5. The method of any one of the preceding Embodiments, wherein the salt of Intermediate I-4 is a bis-hydrochloride salt.

6. The method of any one of the preceding Embodiments, wherein the base of Step (III) is an amine base.

7. The method of any one of the preceding Embodiments, wherein the base of Step (III) is N-methylmorpholine, trimethylamine, triethylamine, N,N-diisopropylethylamine, or N,N-dimethylaniline.

8. The method of any one of the preceding Embodiments, wherein the molar ratio of the base of Step (III) to Intermediate I-4 is about 1:1 to about 10:1.

9. The method of any one of the preceding Embodiments, wherein the molar ratio of the base of Step (III) to Intermediate I-4 is about 3:1 to about 8:1, or preferably about 4.5:1 to about 6.5:1.

10. The method of any one of the preceding Embodiments, wherein the molar ratio of the base of Step (III) to Intermediate I-4 is about 1.0:1, about 1.5:1, about 2.0:1, about 2.5:1, about 3.0:1, about 3.5:1, about 4.0:1, about 4.5:1, about 5.0:1, about 5.5:1, about 6.0:1, about 6.5:1, about 7.0:1, about 7.5:1, about 8.0:1, about 8.5:1, about 9.0:1, about 9.5:1, about 10.0:1.

11. The method of any one of the preceding Embodiments, wherein the coupling reagent of Step (III) is a carbodiimide.

12. The method of any one of the preceding Embodiments, wherein the coupling reagent of Step (III) is 1-Ethyl-3-(3′-dimethylaminopropyl) carbodiimide or a salt thereof.

13. The method of any one of the preceding Embodiments, wherein the coupling reagent of Step (III) is 1-Ethyl-3-(3′-dimethylaminopropyl) carbodiimide hydrochloride.

14. The method of any one of the preceding Embodiments, wherein the molar ratio of the coupling reagent of Step (III) to Intermediate I-4 is about 1:1 to about 2:1.

15. The method of any one of the preceding Embodiments, wherein the molar ratio of the coupling reagent of Step (III) to Intermediate I-4 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

16. The method of any one of the preceding Embodiments, wherein the additive of Step (III) is a racemization inhibitor.

17. The method of any one of the preceding Embodiments, wherein the additive of Step (III) is ethyl 2-(hydroxyimino) cyanoacetate (OXYMA) or 2-hydroxypyridine 1-oxide (HOPO).

18. The method of any one of the preceding Embodiments, wherein the additive of Step (III) is ethyl 2-(hydroxyimino) cyanoacetate (OXYMA).

19. The method of any one of the preceding Embodiments, wherein the additive of Step (III) is 2-hydroxypyridine 1-oxide (HOPO).

20. The method of any one of the preceding Embodiments, wherein the molar ratio of the additive of Step (III) to Intermediate I-4 is about 0.1:1 to about 2:1.

21. The method of any one of the preceding Embodiments, wherein the molar ratio of the additive of Step (III) to Intermediate I-4 is about 0.10:1, about 0.15:1, about 0.2:1, about 0.25:1, about 0.30:1, about 0.35:1, about 0.40:1, about 0.45:1, about 0.50:1, about 0.55:1, about 0.60:1, about 0.65:1, about 0.70:1, about 0.75:1, about 0.80:1, about 0.85:1, about 0.90:1, about 0.95:1, about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, or about 2.00:1.

22. The method of any one of the preceding Embodiments, wherein the solvent of Step (III) is a polar solvent.

23. The method of any one of the preceding Embodiments, wherein the solvent of Step (III) is dimethylacetamide, dimethylsulfoxide, N-methyl-2-pyrrolidone, 2-methyltetrahydrofuran, dichloromethane, or any combination thereof.

24. The method of any one of the preceding Embodiments, wherein Step (III) is conducted at a temperature of about −30° C. to about 30° C., or about −10° C. to about 10° C.

25. The method of any one of the preceding Embodiments, wherein Step (III) is conducted at a temperature of about −30° C., about −25° C., about −20° C., about −15° C., about −10° C., about −5° C., about 0° C., about 5° C., about 10° C., about 15° C., about 20° C., about 25° C., or about 30° C.

26. The method of any one of the preceding Embodiments, wherein Step (III) further comprises Steps (III-ic), (III-iic), (III-iiic), (III-ivc), (III-vc), (III-vic), (III-viic), and (III-viiic):
(III-ic) adding water to the reaction mixture;
(III-iic) adding an agent that induces nucleation to the reaction mixture of Step (III-ic);
(III-iiic) warming the reaction mixture of Step (III-iic) to an elevated temperature;
(III-ivc) agitating the reaction mixture of Step (III-iiic);
(III-vc) adding a second solvent to the reaction mixture of Step (III-ivc);
(III-vic) agitating the reaction mixture of Step (III-vc);
(III-viic) filtering the reaction mixture of Step (III-vic) to produce a cake and rinsing the cake with water; and
(III-viiic) filtering and rinsing the cake of Step (III-viic) with a third solvent.

27. The method of any one of the preceding Embodiments, wherein the agent that induces nucleation of Step (III-iicc) is crystallization promoter, e.g., a seed crystal of Compound A.

28. The method of any one of the preceding Embodiments, wherein the elevated temperature of Step (III-iiic) is about 10° C. to about 35° C.

29. The method of any one of the preceding Embodiments, wherein the elevated temperature of Step (III-iiic) is about 10° C., about 15° C., about 20° C., about 25° C., about 30° C., or about 35° C.

30. The method of any one of the preceding Embodiments, wherein the second solvent of Step (III-vc) is methyl tert-butyl ether.

31. The method of any one of the preceding Embodiments, wherein the third solvent of Step (III-viiic) is methyl tert-butyl ether.

32. The method of any one of the preceding Embodiments, wherein Step (III) further comprises Steps (III-i), (III-ii), and (III-iii):
(III-i) adding water to the reaction mixture;
(III-ii) warming the reaction mixture to an elevated temperature; and
(III-iii) agitating the reaction mixture.

33. The method of any one of the preceding Embodiments, wherein the elevated temperature of Step (III-ii) is about 10° C. to about 30° C.

34. The method of any one of the preceding Embodiments, wherein the elevated temperature of Step (III-ii) is about 10° C., about 15° C., about 20° C., about 25° C., or about 30° C.

35. The method of any one of the preceding Embodiments, wherein Step (III) further comprises Step (III-iv): (III-iv) filtering the mixture and washing the filtrate with water, methyl tert-butyl ether, acetonitrile, or a combination thereof.

36. The method of any one of the preceding Embodiments, comprising washing the filtrate of Step (III-iv) with a combination of water and methyl tert-butyl ether, optionally wherein the ratio of water to methyl tert-butyl ether in the combination is about 1:1.0 (v/v), about 1:1.1 (v/v), about 1:1.2 (v/v), about 1:1.3 (v/v), about 1:1.4 (v/v), or about 1:1.5 (v/v).

37. The method of any one of the preceding Embodiments, further comprising purifying crude Compound A to provide purified Compound A.

38. The method of any one of the preceding Embodiments, wherein the method provides purified, amorphous Compound A.

39. The method of any one of the preceding Embodiments, wherein purifying crude Compound A comprises Step (IV), which comprises Steps (IV-i), (IV-ii), (IV-iii), and (IV-iv):
(IV-i) combining crude Compound A and a solvent to prepare a mixture;
(IV-ii) heating the mixture;
(IV-iii) agitating the mixture; and
(IV-iv) filtering the mixture to provide purified Compound A;
   optionally wherein steps (IV-i), (IV-ii), (IV-iii), and/or (IV-iv) are repeated.

40. The method of any one of the preceding Embodiments, wherein the solvent of Step (IV-i) is water, acetonitrile, or a combination thereof.

41. The method of any one of the preceding Embodiments, wherein the mixture of Step (IV-ii) is heated to a temperature of about 20° C. to about 50° C.

42. The method of any one of the preceding Embodiments, wherein the mixture of Step (IV-ii) is heated to a temperature of about 20° C., about 25° C., about 30° C., about 35° C., about 40° C., about 45° C., or about 50° C.

43. The method of any one of the preceding Embodiments, wherein the purified Compound A of Step (IV-iv) is washed with a combination of water and acetonitrile.

44. The method of any one of the preceding Embodiments, wherein the ratio of water to acetonitrile in the combination used in Step (IV-iv) is about 0.5:1 (v/v), about 0.6:1 (v/v), about 0.7:1 (v/v), about 0.8:1 (v/v), about 0.9:1 (v/v), about 1.0:1 (v/v), about 1.1:1 (v/v), about 1.2:1 (v/v), about 1.3:1 (v/v), about 1.4:1 (v/v), or about 1.5:1 (v/v).

45. The method of any one of the preceding Embodiments, further comprising Step (V): (V) crystallizing Compound A to provide purified crystalline Compound A.

46. The method of any one of the preceding Embodiments, wherein Step (V) further comprises Steps (V-i), (V-ii), (V-iii), (V-iv), (V-v), and (V-vi):
(V-i) combining a first solvent and amorphous Compound A to prepare a mixture;
(V-ii) heating the mixture of Step (V-i);
(V-iii) adding a second solvent to the mixture of Step (V-ii);
(V-iv) adding an agent that induces nucleation to the mixture of Step (V-iii);
(V-v) reducing the volume of the mixture of Step (V-iv); and
(V-vi) agitating the mixture of Step (V-iv).

47. The method of any one of the preceding Embodiments, wherein the first solvent of Step (V-i) is dichloromethane.

48. The method of any one of the preceding Embodiments, wherein the mixture of Step (V-ii) is heated to a temperature of about 20° C. to about 50° C.

49. The method of any one of the preceding Embodiments, wherein the mixture of Step (V-ii) is heated to a temperature of about 20° C., about 25° C., about 30° C., about 35° C., about 40° C., about 45° C., or about 50° C.

50. The method of any one of the preceding Embodiments, wherein the second solvent of Step (V-iii) is an alcohol; optionally wherein the second solvent of Step (V-iii) is methanol or ethanol.

51. The method of any one of the preceding Embodiments, wherein the agent that induces nucleation of Step (V-iv) is a crystallization promoter.

52. The method of any one of the preceding Embodiments, wherein the crystallization promoter of Step (V-iv) is a seed crystal of Compound A.

53. The method of any one of the preceding Embodiments, wherein Step (V) further comprises Steps (V-vii), (V-viii), (V-ix), (V-x), (V-xi), and (V-xii):
(V-vii) adding an additional amount of the second solvent of Step (V-iii) to the mixture of Step (V-vi) to provide a second mixture;
(V-viii) removing the first solvent of Step (V-i) from the second mixture of Step (V-vii) to provide a concentrated second mixture;
(V-ix) agitating the concentrated second mixture of Step (V-viii);
(V-x) cooling the concentrated second mixture of Step (V-ix) to provide a slurry;
(V-xi) filtering the slurry of Step (V-x) to provide a cake; and
(V-xii) washing the cake of Step (V-xi) with the second solvent to provide purified crystalline Compound A.

54. The method of any one of the preceding Embodiments, wherein the first solvent of Step
(V-viii) is removed by distillation.

55. The method of any one of the preceding Embodiments, wherein the first solvent of Step (V-viii) is removed by vacuum distillation.

56. The method of any one of the preceding Embodiments, wherein the first solvent of Step (V-viii) is removed by atmospheric distillation.

57. The method of any one of the preceding Embodiments, wherein the purified crystalline Compound A prepared in Step (V-xii) is at least 95%, at least 96%, at least 97%, at least 98%, or at least 99% pure, as measured by HPLC.

58. The method of any one of the preceding Embodiments, wherein Step (III) further comprises Step (III-A).

59. The method of any one of the preceding Embodiments, wherein Step (III-A) further comprises Step (III-Ai), Step (III-Aii), Step (III-Aiii), Step (III-Aiv), Step (III-Av), Step (III-Avi), Step (III-Avii), Step (III-Aviii), Step (III-Aix), Step (III-Ax), Step (III-Axi), Step (III-Axii), Step (III-Axiii), Step (III-Axiv), Step (III-Axv), Step (III-Axvi), Step (III-Axvii), and Step (III-Axviii):
(III-Ai) adding the reaction mixture comprising crude Compound A to a second reaction mixture comprising water and an agent that induces nucleation to provide a third reaction mixture;
(III-Aii) adding methyl tert-butyl ether to the third reaction mixture of Step (III-Ai);
(III-Aiii) agitating the third reaction mixture of Step (III-Aii) at a temperature of about 15-25° C.;
(III-Aiv) stopping the agitating of the third reaction mixture of Step (III-Aiii) and allowing the phases to separate into layers; (III-Av) removing the bottom layer from the third reaction mixture in Step (III-Aiv) and filtering the solids followed by rinsing, and drying to provide wet, crude Compound A. (III-Avi) charging a separate crystallization reactor with ethanol;
(III-Avii) heating the crystallization reactor of Step (III-Avi) at a temperature of about 15-25° C.;
(III-Aviii) adding dichloromethane and wet, crude Compound A in a separate vessel to provide a fourth reaction mixture;
(III-Aix) stirring the fourth reaction mixture of Step (III-Aviii);
(III-Ax) stopping the stirring of the fourth reaction of Step (III-Aix) and allowing the phases to separate into layers;
(III-Axi) transferring the bottom layer of the fourth reaction mixture of Step (III-Ax) to the crystallization reactor of Step (III-Avii);
(III-Axii) adding a mixture of an agent that induces nucleation in ethanol to the crystallization reactor of step (III-Axi);
(III-Axiii) removing the ethanol and dichloromethane of the crystallization reactor of Step (III-Axii);
(III-Axiv) adding ethanol to the crystallization reactor of Step (III-Axiii) then removing the ethanol;
(III-Axv) repeating Step (III-Axiv) one or more times;
(III-Axvi) cooling the crystallization reactor of Step (III-Axv) to a temperature of about 18-25° C., agitating the crystallization reactor to provide a slurry;
(III-Axvii) filtering the slurry of Step (III-Axvi) and rinsing the resulting cake with ethanol; and
(III-Axviii) drying the cake of Step (III-Axvii) to provide crystalline Compound A.

60. The method of any one of the preceding Embodiments, wherein the agent that induces nucleation of Step (III-Ai) and/or Step (III-Axii) is a crystallization promoter, e.g., a seed crystal of Compound A.

61. The method of any one of the preceding Embodiments, wherein the purified crystalline Compound A prepared in Step (III-Axviii) is at least 95% pure, at least 96% pure, at least 97% pure, at least 98% pure, or at least 99% pure, as measured by HPLC.

62. The method of any one of the preceding Embodiments, wherein Step (III) further comprises Step (III-ia), Step (III-iia), Step (III-iiia), Step (III-iva), Step (III-va), Step (III-via), Step (III-viia), Step (III-viiia), Step (III-ixa), and Step (III-xa):
(III-ia) adding an organic solvent and water and to the reaction mixture comprising crude Compound A, stirring for several minutes, and allowing the reaction mixture to separate into organic and aqueous layers;
(III-iia) removing the organic layer from Step (III-ia) and washing the organic layer with a brine solution;
(III-iiia) removing and filtering the washed organic layer from Step (III-iia);
(III-iva) adding a second solvent and an agent that induces nucleation to the filtrate of Step (III-iiia);
(III-va) removing the second solvent of Step (III-iva) to provide a concentrated mixture; (III-via) adding the second solvent of Step (III-iva) to the concentrated mixture of Step (III-va) and removing the second solvent to provide a concentrated mixture, optionally repeating the process one or more times;
(III-viia) cooling the concentrated mixture of Step (III-via) mixture to a temperature of about 18-25° C. over a period of about one hour;
(III-viiia) agitating the concentrated mixture of Step (III-viia) for about 8 hours or more at a temperature of about 18-25° C. to produce a slurry;
(III-ixa) filtering the slurry of Step (III-viiia) to provide a cake and rinsing the resulting cake with the second solvent of Step (III-iva); and
(III-xa) drying the resulting cake of Step (III-ixa) by removing the second solvent to provide crystalline Compound A.

63. The method of any one of the preceding Embodiments, wherein the organic solvent of Step (III-ia) is dichloromethane.

64. The method of any one of the preceding Embodiments, wherein the brine solution from Step (III-iia) is half saturated brine (i.e., about 13% NaCl (w/v) in water).

65. The method of any one of the preceding Embodiments, wherein the agent that induces nucleation of step (III-iva) is a crystallization promoter, e.g., a seed crystal of Compound A.

66. The method of any one of the preceding Embodiments, wherein the second solvent of Step (III-iva) is an alcohol.

67. The method of any one of the preceding Embodiments, wherein the second solvent of Step (III-iva) is ethanol or methanol.

68. The method of any one of the preceding Embodiments, wherein the second solvent of

Step (III-va), Step (III-via), and Step (III-xa) is removed by distillation.

69. The method of any one of the preceding Embodiments, wherein the second solvent of Step (III-va), Step (III-via), and Step (III-xa) is removed by vacuum distillation.

70. The method of any one of the preceding Embodiments, wherein the second solvent of Step (III-va), Step (III-via), and Step (III-xa) is removed by atmospheric distillation.

71. The method of any one of the preceding Embodiments, wherein the purified crystalline Compound A prepared in Step (III-xa) is at least 95%, at least 96%, at least 97%, at least 98%, or at least 99% pure, as measured by HPLC.

72. A method comprising Step (I):
(I) reductively aminating Intermediate I-1:

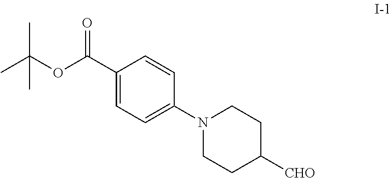 [SMILES: CC(C)(C)OC(=O)c1ccc(N2CCC(C=O)CC2)cc1]

with Intermediate I-2:

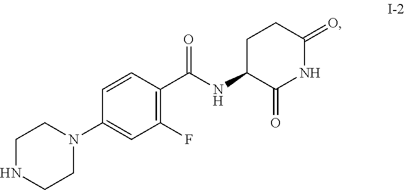 [SMILES: O=C1CC[C@H](NC(=O)c2ccc(N3CCNCC3)cc2F)C(=O)N1]

or a salt thereof,
in a reaction mixture, wherein the reaction mixture comprises a base, a reducing agent, and a solvent to provide wet Intermediate I-3:

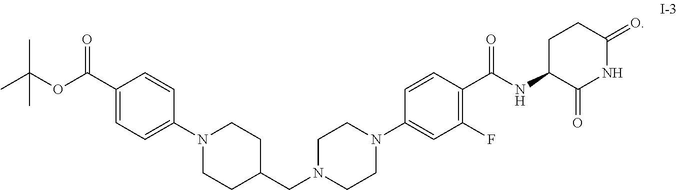 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

73. The method of any one of the preceding Embodiments, wherein the salt of Intermediate I-2 is a hydrochloride salt.

74. The method of any one of the preceding Embodiments, wherein the base of Step (I) is an amine base or a carbonate salt.

75. The method of any one of the preceding Embodiments, wherein the base of Step (I) is N-methylmorpholine, trimethylamine, triethylamine, N,N-diisopropylethylamine, N,N-dimethylaniline, lithium carbonate, sodium carbonate, potassium carbonate, or magnesium carbonate.

76. The method of any one of the preceding Embodiments, wherein the molar ratio of the base of Step (I) to Intermediate I-1 is about 1:1 to about 6:1.

77. The method of any one of the preceding Embodiments, wherein the molar ratio of the base of Step (I) to Intermediate I-1 is about 1.0:1, about 1.5:1, about 2.0:1, about 2.5:1, about 3.0:1, about 3.5:1, about 4.0:1, about 4.5:1, about 5.0:1, about 5.5:1, or about 6.0:1.

78. The method of any one of the preceding Embodiments, wherein the molar ratio of the base of Step (I) to Intermediate I-1 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, about 2.00:1, about 2.05:1, about 2.10:1, about 2:15:1, about 2:20:1, about 2.25:1, about 2.30:1, about 2.35:1, about 2.40:1, about 2.45:1, about 2.50:1, about 2.55:1, about 2.60:1, about 2.65:1, about 2.70:1, about 2.75:1, about 2.80:1, about 2.85:1, about 2.90:1, about 2.95:1, about 3.00:1, about 3.05:1, about 3.10:1, about 3.15:1, about 3.20:1, about 3.25:1, about 3.30:1, about 3.35:1, about 3.40:1, about 3.45:1, about 3.50:1, about 3.55:1, about 3.60:1, about 3.65:1, about 3.70:1, about 3.75:1, about 3.80:1, about 3.85:1, about 3.90:1, about 3.95:1, or about 4.00:1.

79. The method of any one of the preceding Embodiments, wherein the reducing agent of Step (I) is sodium triacetoxyborohydride, sodium borohydride, or sodium cyanoborohydride.

80. The method of any one of the preceding Embodiments, wherein the molar ratio of the reducing agent of Step (I) to Intermediate I-1 is about 1:1 to about 3:1, optionally about 1:1 to about 2:1.

81. The method of any one of the preceding Embodiments, wherein the molar ratio of the reducing agent of Step (I) to Intermediate I-1 is about 1.00:1, about 1.05:1, about 1.10:1, about 1.15:1, about 1.20:1, about 1.25:1, about 1.30:1, about 1.35:1, about 1.40:1, about 1.45:1, about 1.50:1, about 1.55:1, about 1.60:1, about 1.65:1, about 1.70:1, about 1.75:1, about 1.80:1, about 1.85:1, about 1.90:1, about 1.95:1, about 2.00:1, about 2.05:1, or about 2.10:1.

82. The method of any one of the preceding Embodiments, wherein the solvent of Step (I) is a polar solvent.

83. The method of any one of the preceding Embodiments, wherein the solvent of Step (I) is dimethylacetamide, acetonitrile, dimethylsulfoxide, N-methyl-2-pyrrolidone, 2-methyltetrahydrofuran, tetrahydrofuran, 1,4-dioxane, dichloromethane, dimethylformamide, or any combination thereof.

84. The method of any one of the preceding Embodiments, wherein the solvent of Step (I) is dimethylsulfoxide and acetonitrile.

85. The method of any one of the preceding Embodiments, wherein the solvent of Step (I) is dimethylacetamide.

86. The method of any one of the preceding Embodiments, wherein the solvent of Step (I) is acetonitrile.

87. The method of any one of the preceding Embodiments, wherein the solvent of Step (I) is dimethylsulfoxide.

88. The method of any one of the preceding Embodiments, wherein the solvent of Step (I) is 2-methyltetrahydrofuran.

89. The method of any one of the preceding Embodiments, wherein the solvent of Step (I) is 1,4-dioxane.

90. The method of any one of the preceding Embodiments, wherein Step (I) is conducted at a temperature of about −30° C. to about 30° C.; optionally about −10° C. to about 10° C.

91. The method of any one of the preceding Embodiments, wherein Step (I) is conducted at a temperature of about −30° C., about −25° C., about −20° C., about −15° C., about −10° C., about −5° C., about 0° C., about 5° C., about 10° C., about 15° C., about 20° C., about 25° C., or about 30° C.

92. The method of any one of the preceding Embodiments, wherein Step (I) further comprises Step (I-i) and optionally Step (I-A): (I-i) filtering and washing the wet Intermediate I-3 with a second solvent;
(I-A) azeotropically drying wet Intermediate I-3 using a third solvent, to provide dried Intermediate I-3.

93. The method of any one of the preceding Embodiments, wherein the second solvent of Step (I-i) is (i) water; or (ii) water and acetonitrile.

94. The method of any one of the preceding Embodiments, wherein the third solvent of Step (I-A) is toluene.

95. The method of any one of the preceding Embodiments, wherein Step (I) further comprises Steps (I-ia), (I-iia), (I-iiia), (I-iva), (I-va), (I-via), and (I-viia):
(I-ia) adding a second solvent to the reaction mixture;
(I-iia) adding a third solvent to the reaction mixture of Step (I-ia);
(I-iiia) adding an agent that induces nucleation to the reaction mixture of Step (I-iia);
(I-iva) adding water to the reaction mixture of Step (I-iiia);
(I-va) cooling the reaction mixture of Step (I-iva);
(I-via) agitating the reaction mixture of Step (I-va); and
(I-viia) filtering the reaction mixture of Step (I-va) to provide a cake and rinsing the cake with a fourth solvent one or more times.

96. The method of any one of the preceding Embodiments, wherein the second solvent of Step (I-ia) is (i) water; or (ii) water and acetonitrile.

97. The method of any one of the preceding Embodiments, wherein the agent that induces nucleation of Step (I-iiia) is a crystallization promoter.

98. The method of any one of the preceding Embodiments, wherein the crystallization promoter of Step (I-iiia) is a seed crystal of Intermediate I-3.

99. The method of any one of the preceding Embodiments, wherein Step (I-va) is conducted at a temperature of about 5° C. to about 25° C.

100. The method of any one of the preceding Embodiments, wherein the dried Intermediate I-3 prepared in Step (I-viia″) is at least 95%, at least 96%, at least 97%, at least 98%, or at least 99% pure, as measured by HPLC.

101. A method comprising Step (II):
(II) reacting Intermediate I-3:

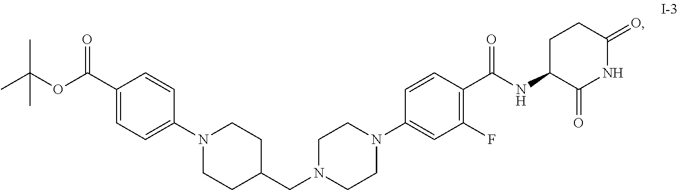 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid in a reaction mixture to provide Intermediate I-4:

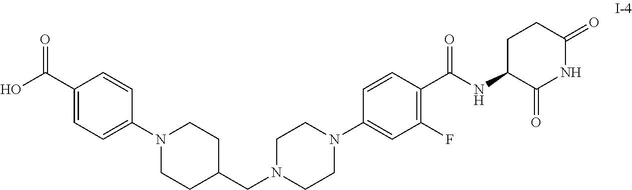 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, wherein the reaction mixture comprises a first solvent.

102. The method of any one of the preceding Embodiments, wherein Step (II) provides a hydrochloride salt of Intermediate I-4.

103. The method of any one of the preceding Embodiments, wherein Step (II) provides a bis-hydrochloride salt of Intermediate I-4.

104. The method of any one of the preceding Embodiments, wherein the acid of Step (II) is an organic acid.

105. The method of any one of the preceding Embodiments, wherein the organic acid is trifluoroacetic acid.

106. The method of any one of the preceding Embodiments, wherein the first solvent of Step (II) is dichloromethane.

107. The method of any one of the preceding Embodiments, wherein Step (II) is conducted at a temperature of about 20° C. to about 50° C.

108. The method of any one of the preceding Embodiments, wherein Step (II) is conducted at a temperature of about 20° C., about 25° C., about 30° C., about 35° C., about 40° C., about 45° C., or about 50° C.

109. The method of any one of the preceding Embodiments, wherein Step (II) further comprises Steps (II-i), (II-ii), (II-iii), (II-iv), and (II-v):
(II-i) adding a second solvent to the reaction mixture of Step (II);
(II-ii) distillatively removing the first solvent of Step (II) and second solvent of Step (II-i) from the reaction mixture of Step (II-i);
(II-iii) adding an additional amount of the second solvent of Step (II-i) to the reaction mixture of Step (II-ii);
(II-iv) adding an aqueous basic solution to the reaction mixture of Step (II-iii) such that the pH of the reaction mixture is about pH 5.5 to about pH 6.5; and
(II-v) filtering the reaction mixture of Step (II-iv) and washing the resulting cake with water to provide wet Intermediate I-4.

110. The method of any one of the preceding Embodiments, wherein the second solvent of Step (II-i) is acetonitrile.

111. The method of any one of the preceding Embodiments, wherein the aqueous basic solution of Step (II-iv) comprises sodium bicarbonate.

112. The method of any one of the preceding Embodiments, wherein Step (II) further comprises Step (II-vi) and optionally Step (II-vii):
(II-vi) filtering and washing the wet Intermediate I-4 with a second solvent; and/or (II-vii) azeotropically drying wet Intermediate I-4 using a third solvent, to provide dried Intermediate I-4.

113. The method of any one of the preceding Embodiments, wherein the second solvent of Step (II-vi) is (i) water; or (ii) water and acetonitrile.

114. The method of any one of the preceding Embodiments, wherein the third solvent of Step (II-vii) is toluene.

115. The method of any one of the preceding Embodiments, wherein Step (II) further comprises Steps (II-i′), (II-ii′), (II-iii′), (II-iv′), and (II-v′):
(II-i′) adding a second solvent to the reaction mixture of Step (II);
(II-ii′) distillatively removing the first solvent of Step (II) and the second solvent of Step
(II-i′) from the reaction mixture of Step (II-i′);
(II-iii′) adding an additional amount of the second solvent of Step (II-i′) to the reaction mixture of Step (II-ii′);
(II-iv′) adding an aqueous acidic solution to the reaction mixture of Step (II-iii′); and (II-v′) filtering the reaction mixture of Step (II-iv′) and washing the resulting cake with water to provide wet Intermediate I-4.

116. The method of any one of the preceding Embodiments, wherein the second solvent of Step (II-i′) is acetonitrile.

117. The method of any one of the preceding Embodiments, wherein the aqueous acidic solution of Step (II-iv′) comprises hydrochloric acid.

118. The method of any one of the preceding Embodiments, wherein Step (II) provides a bis-hydrochloride salt of Intermediate I-4.

119. The method of any one of the preceding Embodiments, wherein Intermediate I-4 is at least 95%, at least 96%, at least 97%, at least 98%, or at least 99% pure, as measured by HPLC.

120. The method of Embodiment 1, wherein Intermediate I-4:

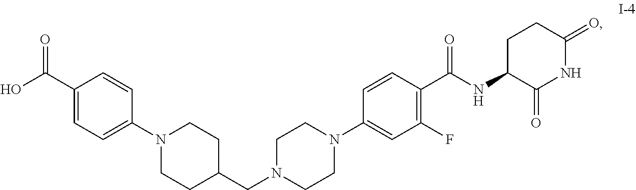 [SMILES: O=C1CC[C@H](NC(=O)c2ccc(N3CCN(CC4CCN(c5ccc(C(=O)O)cc5)CC4)CC3)cc2F)C(=O)N1]

or a salt thereof, is prepared by a method comprising reacting Intermediate I-3:

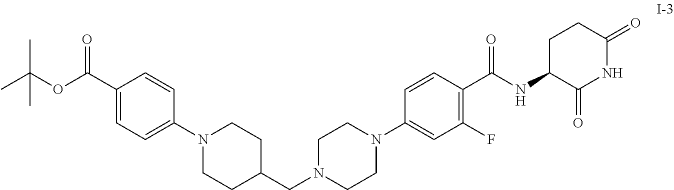 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof, with an acid in a reaction mixture comprising a first solvent.

121. The method of any one of the preceding Embodiments, wherein Intermediate I-3 is prepared by a method comprising reductively aminating Intermediate I-1:

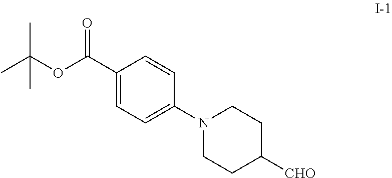 [SMILES: CC(C)(C)OC(=O)c1ccc(N2CCC(C=O)CC2)cc1]

with Intermediate I-2:

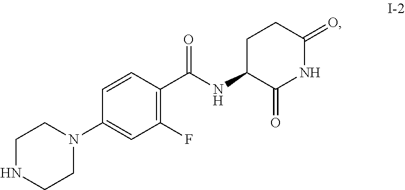 [SMILES: O=C1CC[C@H](NC(=O)c2ccc(N3CCNCC3)cc2F)C(=O)N1]

or a salt thereof,
in a reaction mixture comprising a base, a reducing agent, and a solvent to provide wet Intermediate I-3:

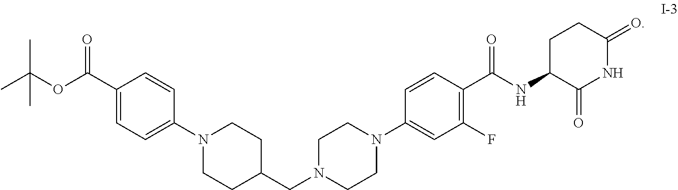 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

122. A method for preparing Compound A, wherein the method comprises:
(a) reacting Intermediate I-1:

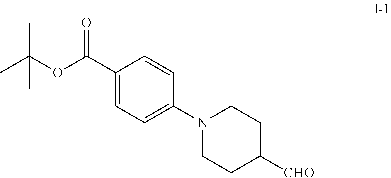 [SMILES: CC(C)(C)OC(=O)c1ccc(N2CCC(C=O)CC2)cc1]

with Intermediate I-2:

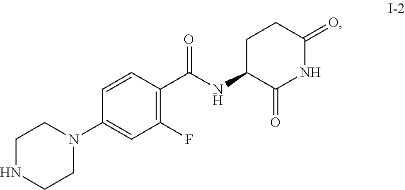 [SMILES: O=C1CC[C@H](NC(=O)c2ccc(N3CCNCC3)cc2F)C(=O)N1]

or a salt thereof,
in a first reaction mixture comprising a base, a reducing agent, and a first solvent to provide wet Intermediate I-3:

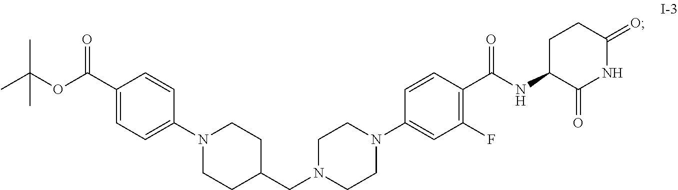 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

(b) drying wet Intermediate I-3;
(c) reacting dried Intermediate I-3:

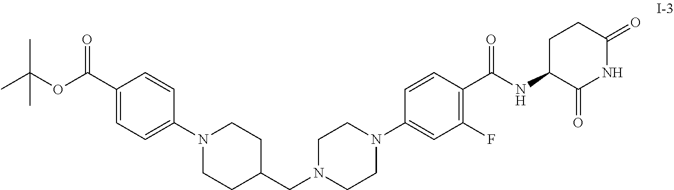 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

or a salt thereof,
with an acid in a second reaction mixture to provide Intermediate I-4:

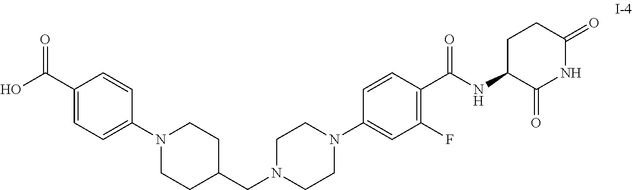 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
wherein the second reaction mixture comprises a second solvent;
(d) reacting Intermediate I-4

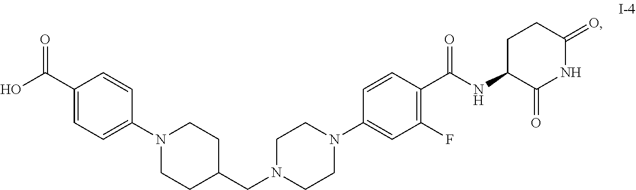 [SMILES: O=C1CC[C@H](NC(=O)c2ccc(N3CCN(CC4CCN(c5ccc(C(=O)O)cc5)CC4)CC3)cc2F)C(=O)N1]

or a salt thereof,
with Intermediate I-5:

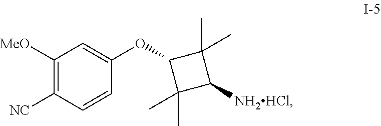

in a third reaction mixture, wherein the third reaction mixture comprises a base, a coupling reagent, and an additive in a third solvent to provide crude Compound A:

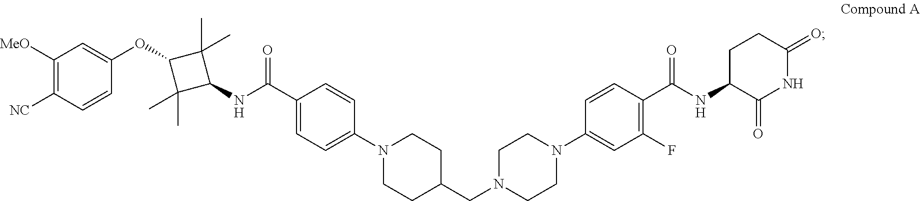 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and, optionally,
wherein crude Compound A is purified by recrystallization.

123. A compound which is:
tert-butyl(S)-4-(4-((4-(4-((2,6-dioxopiperidin-3-yl) carbamoyl)-3-fluorophenyl) piperazin-1-yl)methyl) piperidin-1-yl)benzoate (Intermediate I-3):

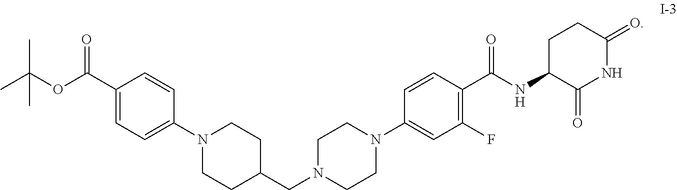 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

124. A preparation of Intermediate I-3, wherein the Intermediate I-3 in the preparation has a purity of greater than about 95%, greater than about 96%, or greater than about 97%, as measured by HPLC.

125. A compound which is:
(S)-4-(4-((4-(4-((2,6-dioxopiperidin-3-yl) carbamoyl)-3-fluorophenyl) piperazin-1-yl)methyl) piperidin-1-yl)benzoic acid (Intermediate I-4),

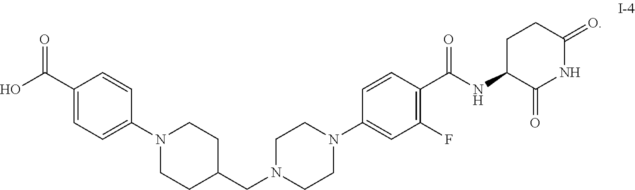 [SMILES: O=C1CC[C@H](NC(=O)c2ccc(N3CCN(CC4CCN(c5ccc(C(=O)O)cc5)CC4)CC3)cc2F)C(=O)N1]

126. A preparation of Intermediate I-4, wherein Intermediate I-4 in the preparation has a purity of greater than about 95%, greater than about 96%, greater than about 97%, or greater than about 98%, as measured by HPLC.

127. A preparation of 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide (Compound A),

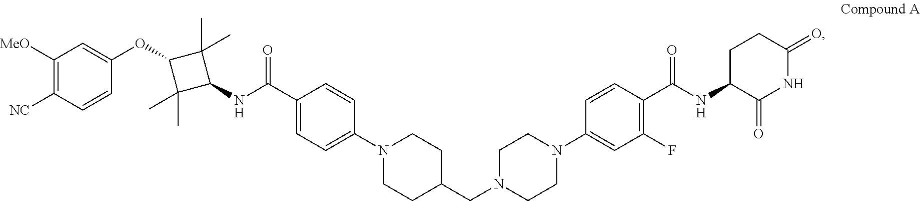 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein Compound A in the preparation has a purity of greater than about 95%, greater than about 96%, greater than about 97%, or greater than about 98%, as measured by HPLC.

128. A preparation of Compound A, wherein Compound A in the preparation has a purity of greater than about 98%, and comprises less than about 0.2%, less than about 0.3%, less than about 0.4%, or less than about 0.5% of impurity Intermediate I-3:

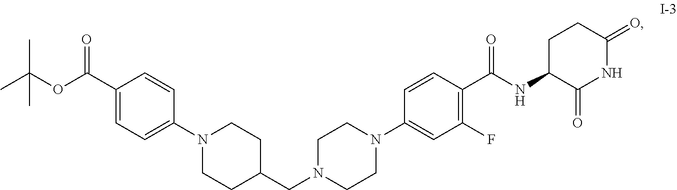 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

as measured by HPLC.

129. A preparation of Compound A, wherein Compound A in the preparation has a purity of greater than about 98%, and comprises less than about 0.2%, less than about 0.3%, less than about 0.4%, or less than about 0.5% of impurity Intermediate I-4:

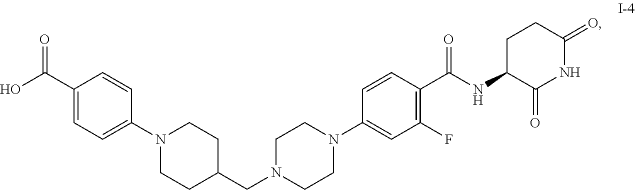 [SMILES: O=C1CC[C@H](NC(=O)c2ccc(N3CCN(CC4CCN(c5ccc(C(=O)O)cc5)CC4)CC3)cc2F)C(=O)N1]

as measured by HPLC.

130. A preparation of Compound A, wherein Compound A in the preparation has a purity of greater than about 98%, and comprises less than about 0.1%, less than about 0.2%, less than about 0.3%, less than about 0.4%, or less than about 0.5% of impurity:

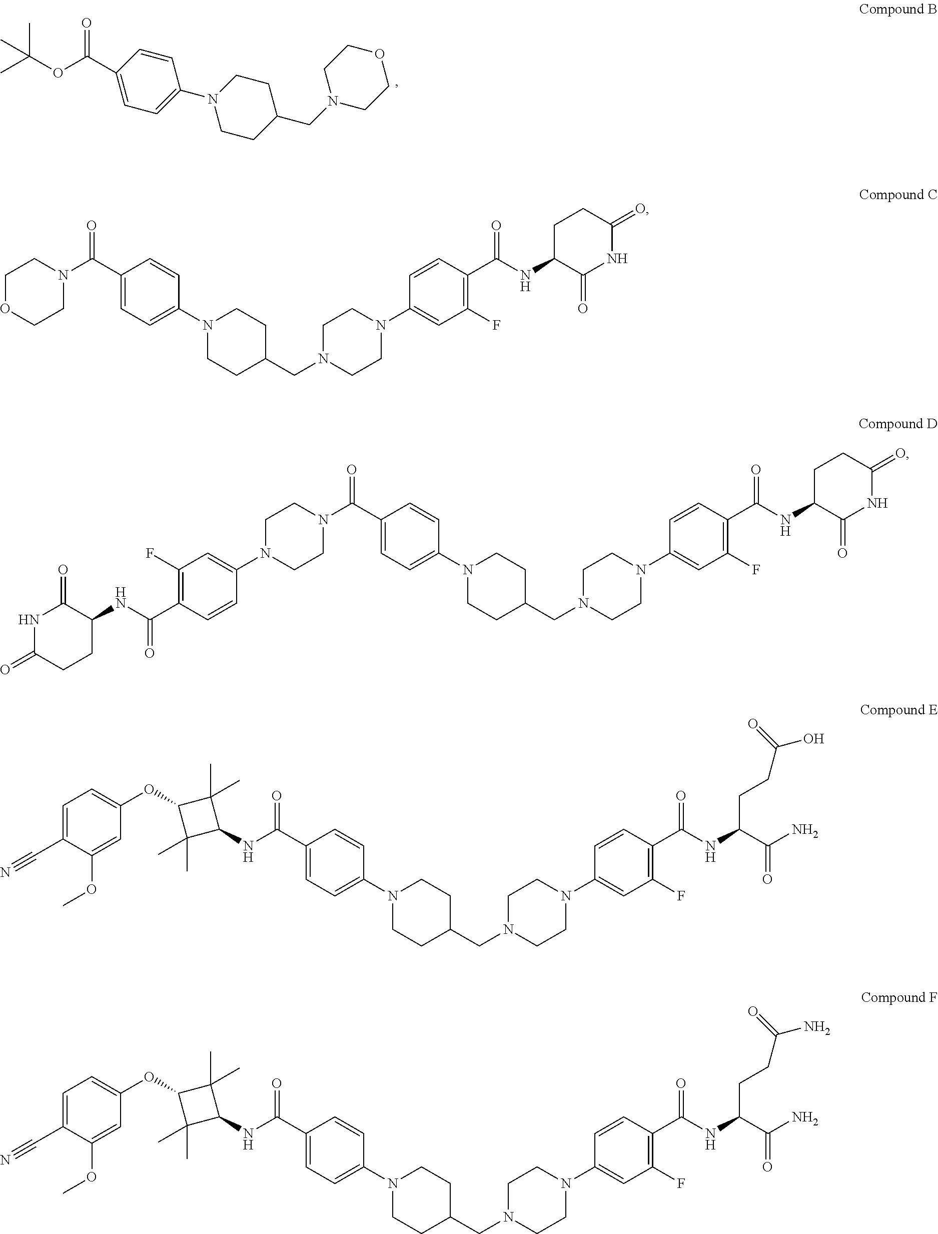 [SMILES: CC(C)(C)OC(=O)c1ccc(N2CCC(CN3CCOCC3)CC2)cc1 ; O=C1CC[C@H](NC(=O)c2ccc(N3CCN(CC4CCN(c5ccc(C(=O)N6CCOCC6)cc5)CC4)CC3)cc2F)C(=O)N1 ; O=C1CC[C@H](NC(=O)c2ccc(N3CCN(CC4CCN(c5ccc(C(=O)N6CCN(c7ccc(C(=O)N[C@H]8CCC(=O)NC8=O)c(F)c7)CC6)cc5)CC4)CC3)cc2F)C(=O)N1 ; COc1cc(O[C@H]2C(C)(C)[C@H](NC(=O)c3ccc(N4CCC(CN5CCN(c6ccc(C(=O)N[C@@H](CCC(=O)O)C(N)=O)c(F)c6)CC5)CC4)cc3)C2(C)C)ccc1C#N ; COc1cc(O[C@H]2C(C)(C)[C@H](NC(=O)c3ccc(N4CCC(CN5CCN(c6ccc(C(=O)N[C@@H](CCC(N)=O)C(N)=O)c(F)c6)CC5)CC4)cc3)C2(C)C)ccc1C#N]

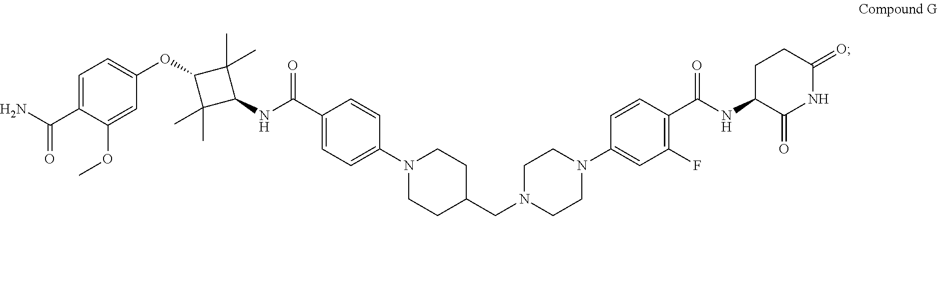 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C(N)=O]

or any combination thereof, as measured by HPLC.

1. A method of preparing Compound A, wherein the method comprises Step (III), wherein Step (III) comprises:
(III) reacting Intermediate I-4: 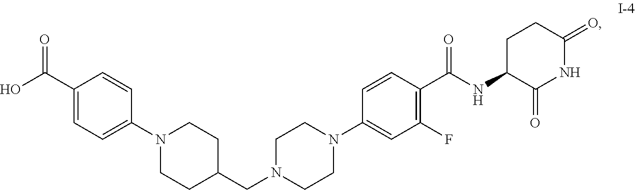 [SMILES: O=C1CC[C@H](NC(=O)c2ccc(N3CCN(CC4CCN(c5ccc(C(=O)O)cc5)CC4)CC3)cc2F)C(=O)N1]
or a salt thereof,
with Intermediate 1-5: 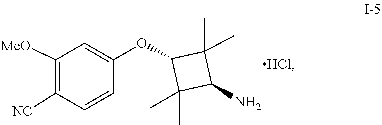 [SMILES: COc1cc(O[C@H]2C(C)(C)[C@H](N)C2(C)C)ccc1C#N]
in a reaction mixture, wherein the reaction mixture comprises a base, a coupling reagent, and an additive in a solvent to provide Compound A:

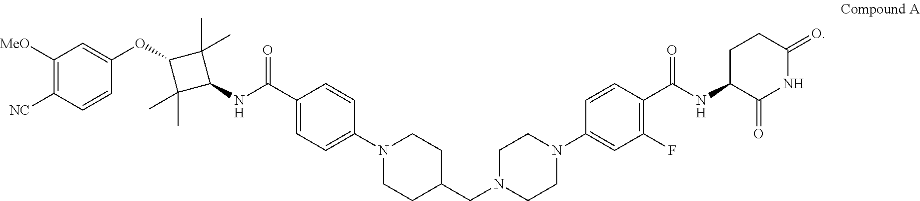 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

2-14. (canceled)

15. The method of claim 1, further comprising Step (V):
(V) crystallizing Compound A to provide purified crystalline Compound A.

16. A method of preparing Intermediate I-3, wherein the method comprises Step (I), wherein Step (I) comprises:
(I) reductively aminating Intermediate I-1: 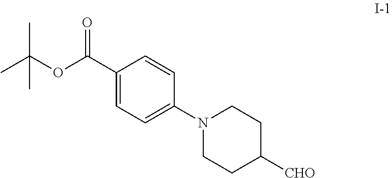 [SMILES: CC(C)(C)OC(=O)c1ccc(N2CCC(C=O)CC2)cc1]
with Intermediate I-2: 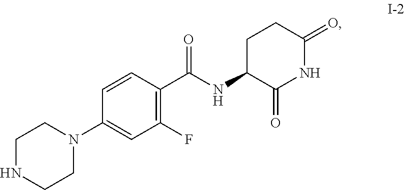 [SMILES: O=C1CC[C@H](NC(=O)c2ccc(N3CCNCC3)cc2F)C(=O)N1]
or a salt thereof,
in a reaction mixture, wherein the reaction mixture comprises a base, a reducing agent, and a solvent to provide wet Intermediate 1-3:

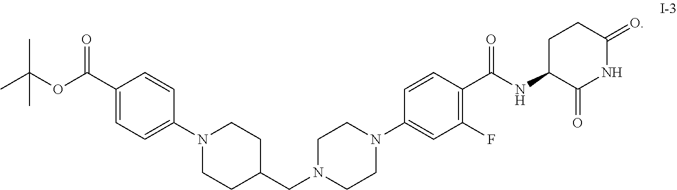 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

17. The method of claim 16, wherein the salt of Intermediate I-2 is a hydrochloride salt.

18. The method of claim 16, wherein the base of Step (I) is an amine base or a carbonate salt.

19. The method of claim 16, wherein the molar ratio of the base of Step (I) to Intermediate I-1 is about 1:1 to about 6:1.

20. The method of claim 16, wherein the molar ratio of the reducing agent of Step (I) to Intermediate I-1 is about 1:1 to about 3:1, optionally about 1:1 to about 2:1.

21. The method of claim 16, wherein the solvent of Step (I) is a polar solvent.

22. The method of claim 16, wherein Step (I) is conducted at a temperature of about −30° C. to about 30° C.

23. A method of preparing Compound I-4, wherein the method comprises Step (II), wherein Step (II) comprises:
(II) reacting Intermediate 1-3: 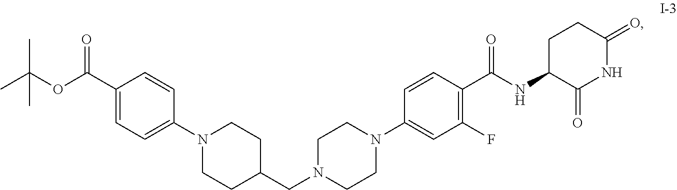 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]
or a salt thereof, with an acid in a
reaction mixture to provide Intermediate I-4: 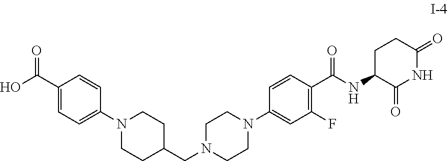 [SMILES: O=C1CC[C@H](NC(=O)c2ccc(N3CCN(CC4CCN(c5ccc(C(=O)O)cc5)CC4)CC3)cc2F)C(=O)N1]
or a salt thereof, wherein the reaction mixture comprises a first solvent.

24. The method of claim 23, wherein Step (II) provides a hydrochloride salt of Intermediate 1-4.

25. The method of claim 23, wherein the acid of Step (II) is an organic acid.

26. The method of claim 23, wherein the first solvent of Step (II) is dichloromethane.

27. The method of claim 23, wherein Step (II) is conducted at a temperature of about 20° C. to about 50° C.

28. The method of claim 23, wherein Intermediate I-4 is at least 95% pure, as measured by HPLC.

29. The method of claim 1, wherein Intermediate I-4: 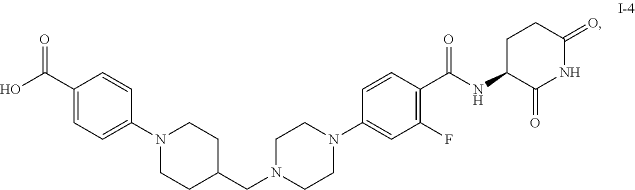 [SMILES: O=C1CC[C@H](NC(=O)c2ccc(N3CCN(CC4CCN(c5ccc(C(=O)O)cc5)CC4)CC3)cc2F)C(=O)N1]
or a salt thereof, is prepared by a method comprising reacting Intermediate I-3: 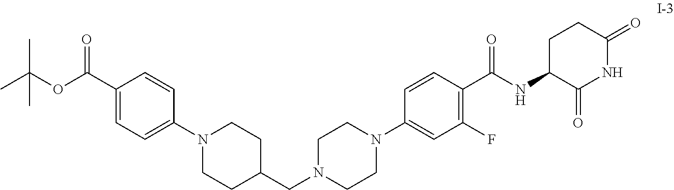 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]
or a salt thereof, with an acid in a reaction mixture comprising a first solvent.

30. The method of claim 29, wherein Intermediate I-3 is prepared by a method comprising reductively aminating Intermediate I-1: 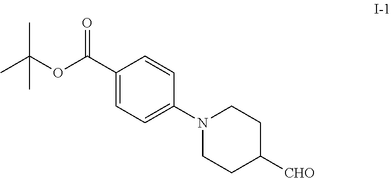 [SMILES: CC(C)(C)OC(=O)c1ccc(N2CCC(C=O)CC2)cc1]
with Intermediate 1-2: 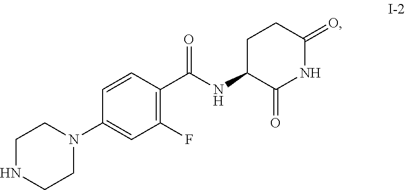 [SMILES: O=C1CC[C@H](NC(=O)c2ccc(N3CCNCC3)cc2F)C(=O)N1]
or a salt thereof,
in a reaction mixture comprising a base, a reducing agent, and a solvent to provide wet Intermediate 1-3:

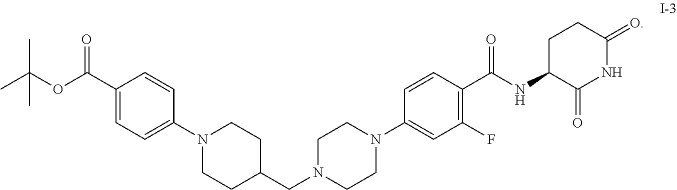 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]

31. A method of preparing Compound A, wherein the method comprises:
(a) reacting Intermediate I-1: 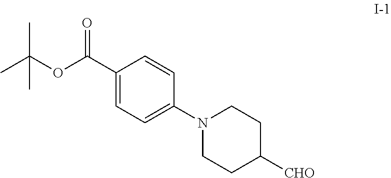 [SMILES: CC(C)(C)OC(=O)c1ccc(N2CCC(C=O)CC2)cc1]
with Intermediate 1-2: 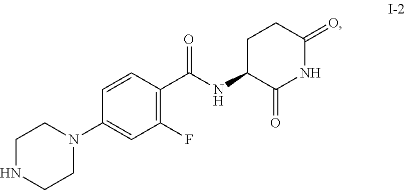 [SMILES: O=C1CC[C@H](NC(=O)c2ccc(N3CCNCC3)cc2F)C(=O)N1]
or a salt thereof,
in a first reaction mixture comprising a base, a reducing agent, and a first solvent to provide wet Intermediate I-3: 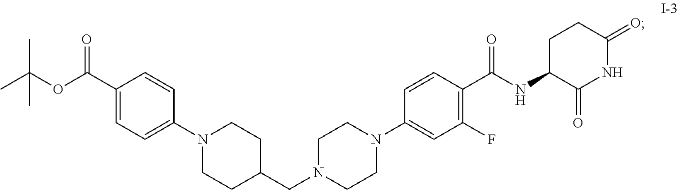 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]
(b) drying wet Intermediate I-3;
(c) reacting dried Intermediate 1-3: 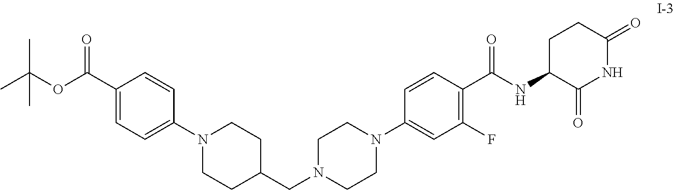 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]
or a salt thereof,
with an acid in a second reaction mixture to provide Intermediate I-4: 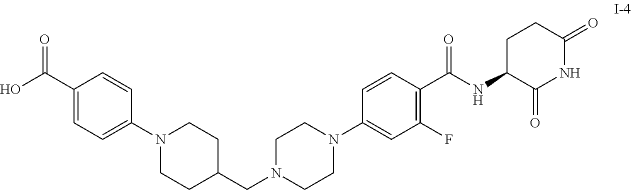 [SMILES: O=C1CC[C@H](NC(=O)c2ccc(N3CCN(CC4CCN(c5ccc(C(=O)O)cc5)CC4)CC3)cc2F)C(=O)N1]
or a salt thereof,
wherein the second reaction mixture comprises a second solvent;
(d) reacting Intermediate I-4 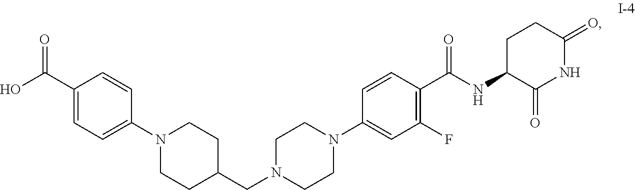 [SMILES: O=C1CC[C@H](NC(=O)c2ccc(N3CCN(CC4CCN(c5ccc(C(=O)O)cc5)CC4)CC3)cc2F)C(=O)N1]
or a salt thereof,
with Intermediate 1-5: 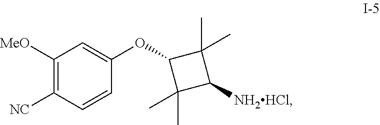
in a third reaction mixture, wherein the third reaction mixture comprises a base, a coupling reagent, and an additive in a third solvent to provide crude Compound A: 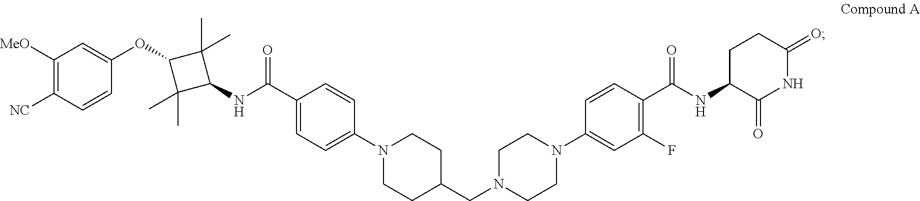 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
and, optionally, wherein crude Compound A is purified by recrystallization.

32. A preparation of 4-(4-((1-(4-(((1r,3r)-3-(4-cyano-3-methoxyphenoxy)-2,2,4,4-tetramethylcyclobutyl) carbamoyl)phenyl) piperidin-4-yl)methyl) piperazin-1-yl)-N-((S)-2,6-dioxopiperidin-3-yl)-2-fluorobenzamide (Compound A), 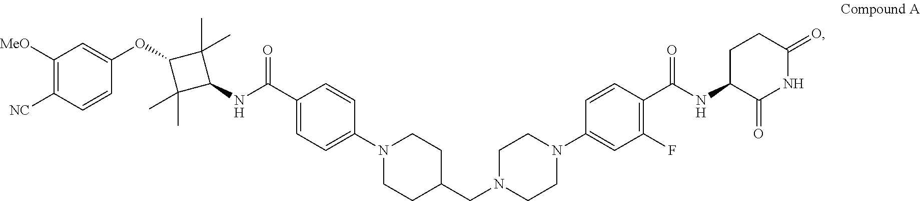 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
wherein Compound A in the preparation has a purity of greater than about 95% as measured by HPLC.

33. The preparation of claim 32, wherein Compound A in the preparation has a purity of greater than about 98%, and comprises less than about 0.5% of impurity Intermediate I-3: 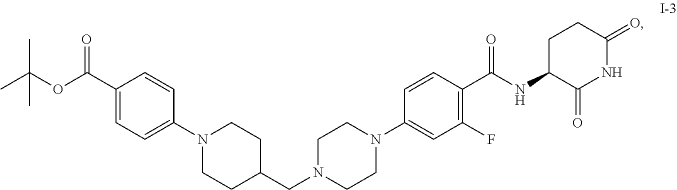 [SMILES: CC(C)(C)OC(=O)c1ccc(N2CCC(CN3CCN(c4ccc(C(=O)N[C@H]5CCC(=O)NC5=O)c(F)c4)CC3)CC2)cc1]
as measured by HPLC.

34. The preparation of claim 32, wherein Compound A in the preparation has a purity of greater than about 98%, and comprises less than about 0.5% of impurity Intermediate I-4: 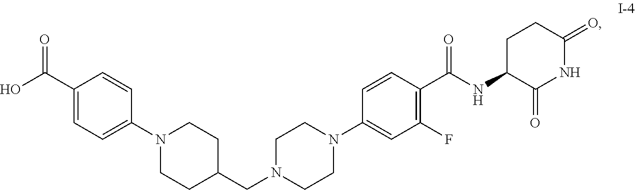 [SMILES: O=C1CC[C@H](NC(=O)c2ccc(N3CCN(CC4CCN(c5ccc(C(=O)O)cc5)CC4)CC3)cc2F)C(=O)N1]
as measured by HPLC.

35. The preparation of claim 32, wherein Compound A in the preparation has a purity of greater than about 98%, and comprises less than about 0.5% of: 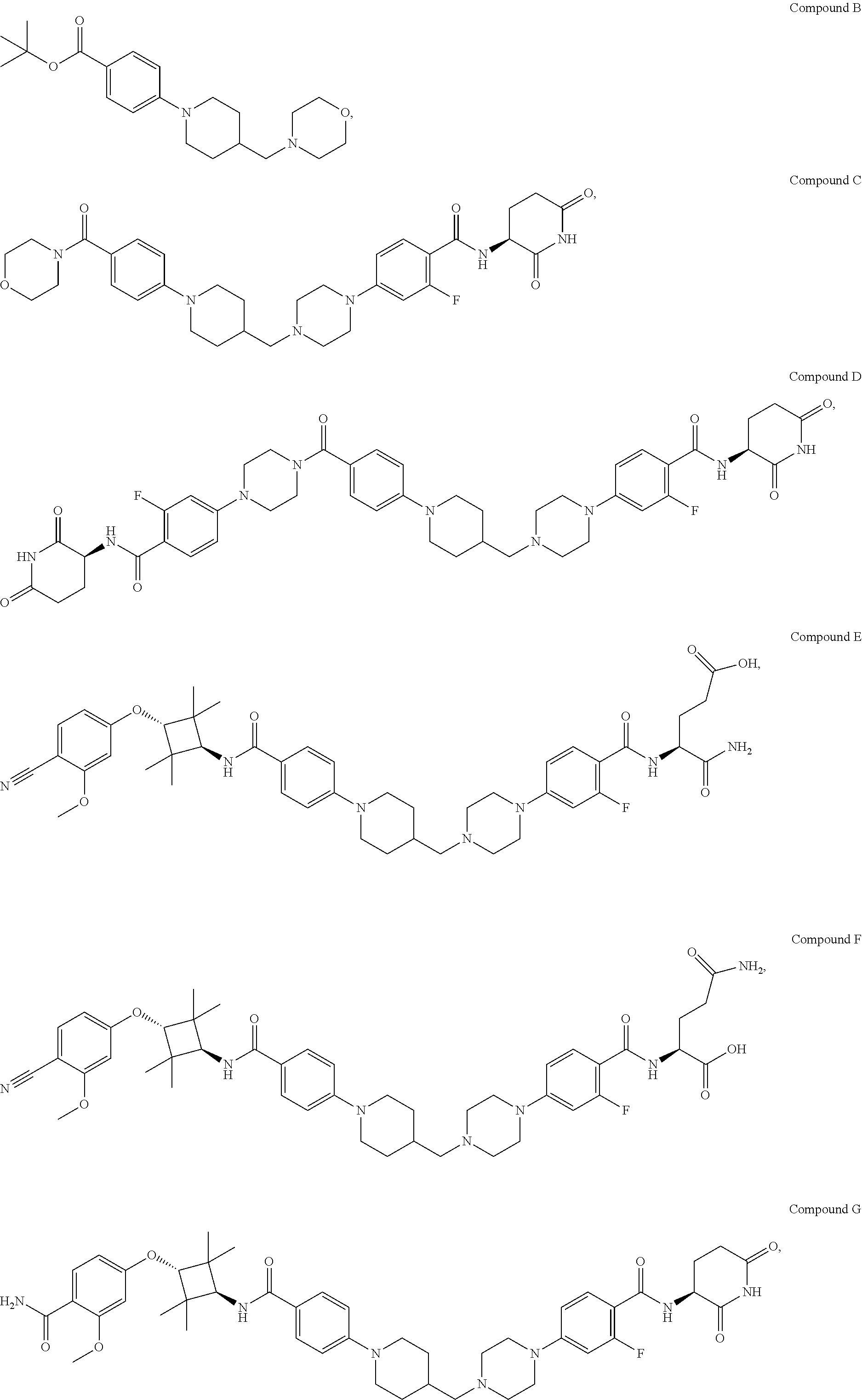
or a combination thereof, as measured by HPLC.
